(19) United States
(12) Patent Application Publication
(10) Pub. No.: US 2026/0182604 A1
(43) Pub. Date: Jul. 2, 2026

(54) COMPOSITIONS AND METHODS TO INCREASE FEED EFFICIENCY IN ANIMALS

(71) Applicant: Church & Dwight Co., Inc., Princeton, NJ (US)

(72) Inventors: Alexandra SMITH, Greendale, WI (US); Thomas REHBERGER, Wauwatosa, WI (US); Sangita JALUKAR, Mason City, IA (US); Seth WENNER, Menomonee Falls, WI (US)

(73) Assignee: Church & Dwight Co., Inc., Princeton, NJ (US)

(21) Appl. No.: 19/131,508

(22) PCT Filed: Nov. 22, 2023
(86) PCT No.: PCT/US2023/080874

§ 371 Date: May 20, 2025

Related U.S. Application Data
(60) Provisional application No. 63/384,713, filed on Nov. 22, 2022.

(51) Int. Cl. A23K 10/18; A23K 20/189; A23K 50/75; C12N 1/205; C12R 1/125

(52) U.S. Cl. CPC A23K 10/18; A23K 20/189; A23K 50/75; C12N 1/205; C12R 2001/125

(57) ABSTRACT

Bacillus strain compositions and methods for converting (i) one or more polysaccharides into short chain polysaccharides and/or monosaccharides and/or (ii) one or more proteins into short chain polypeptides and/or amino acids are provided. The Bacillus strain composition can comprise Bacillus subtilis 750 or an active variant thereof, and optionally at least one of Bacillus subtilis 747, Bacillus subtilis 839, Bacillus subtilis 1781, Bacillus subtilis 1999, Bacillus subtilis 2018, or an active variant thereof. Compositions provided herein can be fed to animals in an effective amount to increase digestibility of one or more polysaccharides and/or one or more proteins; increase nutrition efficiency and/or energy partitioning; increase nutrient absorption; enhance gut immunity and/or gut barrier integrity; and/or inhibit a pathogen in the gastrointestinal tract of the animal.

### RELATED APPLICATIONS

This application claims priority to U.S. Provisional Application No. 63/384,713 filed on Nov. 22, 2022, the content of which is incorporated herein by reference in its entirety.

### FIELD OF THE INVENTION

The invention relates to microbial compositions and methods for improving energy partitioning and/or increasing digestibility of polysaccharides and/or proteins in animals.

### BACKGROUND OF THE INVENTION

Providing sufficient nutrients to farm animals to meet metabolic requirements is important for animal health and commercial benefit. Insufficient energy intake is a common stressor in animals and a cause of reduction in animal performance. In addition to the impact on the metabolic activity of the body, nutritional stress can cause disturbance in the gut microecology and gut immunity and can lead to dysbiosis and disruption in digestion and utilization of nutrients in the gut. On the other hand, there is a continuous scarcity of raw materials and consequential increase in price of animal food (e.g., feed, forage, or fodder). For example, feed costs account for 70% of total costs in broiler production. As a result, increase in feed ingredient prices negatively impact profitability.

Accordingly, improvement in feed efficiency or feed conversion ratio is an ongoing need. Improving nutrient utilization in animals, which can lead to reduction in the amount and cost of feed, forage, or fodder required to maintain animal performance, could offer important animal health and commercial advantages.

### BRIEF SUMMARY OF THE INVENTION

Bacillus strain compositions and methods for converting (i) one or more polysaccharides into short chain polysaccharides and/or monosaccharides and/or (ii) one or more proteins into short chain polypeptides and/or amino acids are provided. The Bacillus strain composition can comprise a bacterial cell, a spore, a forespore, and/or a combination of cells, spores, and/or forespores of Bacillus subtilis 747 and Bacillus subtilis 750, or an active variant thereof. Compositions provided herein can be fed to animals in an effective amount to increase digestibility of one or more polysaccharides and/or one or more proteins; increase production of short chain polysaccharides, monosaccharides, short chain polypeptides, and/or amino acids; increase nutrition efficiency; improve energy partitioning; enhance gut immunity or gut barrier integrity; and/or inhibit a pathogen in the gastrointestinal tract of the animal.

In one aspect, the present disclosure provides a Bacillus strain composition comprising a bacterial cell, a spore, a forespore, and/or a combination of cells, spores, and/or forespores of Bacillus strain Bacillus subtilis 750 or an active variant thereof, wherein an effective amount of said Bacillus strain composition increases conversion of (i) one or more polysaccharides into short chain polysaccharides and/or monosaccharides and/or (ii) one or more proteins into short chain polypeptides and/or amino acids, relative to the absence of said effective amount of said Bacillus strain composition.

In some embodiments, the Bacillus strain composition further comprises a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In some embodiments, the Bacillus strain composition comprises said at least one of Bacillus subtilis 747, Bacillus subtilis 839, Bacillus subtilis 1781, Bacillus subtilis 1999, Bacillus subtilis 2018, or an active variant thereof and said Bacillus subtilis 750 or active variant thereof in equal proportions.

In some embodiments, the Bacillus strain composition further comprises a bacterial cell, a spore, a forespore, and/or a combination of cells, spores, and/or forespores of said Bacillus subtilis 747 or active variant thereof. In some embodiments, the Bacillus strain composition comprises said Bacillus subtilis 747 or active variant thereof and said Bacillus subtilis 750 or active variant thereof in equal proportions.

In some embodiments, said bacterial cell, spore, forespore, and/or combination of cells, spores, and/or forespores of said Bacillus strain(s) is present in the composition at about 1×10⁵ CFU/gram to about 1×10¹⁰ CFU/gram or at about 1×10⁵ CFU/ml to about 1×10¹⁰ CFU/ml.

In some embodiments, said bacterial cell, spore, forespore, and/or combination of cells, spores, and/or forespores of said Bacillus subtilis 747 or active variant thereof and said Bacillus subtilis 750 or active variant thereof is present in the composition at about 1×10⁵ CFU/gram to about 1×10¹⁰ CFU/gram or at about 1×10⁵ CFU/ml to about 1×10¹⁰ CFU/ml.

In some embodiments, at least one of said Bacillus strains in the Bacillus strain composition is a powdered, lyophilized strain. Bacillus strain composition, Bacillus strain composition further comprises a cryoprotectant. In some embodiments, the Bacillus strain composition comprises a preservative.

In some embodiments, the Bacillus strain composition further comprises said one or more polysaccharides and/or said one or more proteins. In some embodiments, the Bacillus strain composition comprises feed, forage, or fodder comprising said one or more polysaccharides and/or said one or more proteins.

In one aspect, the present disclosure provides a Bacillus strain composition comprising one or more polysaccharides and/or one or more proteins and a Bacillus strain, said Bacillus strain comprising a bacterial cell, a spore, a forespore, and/or a combination of cells, spores, and/or forespores of Bacillus subtilis 750 or an active variant thereof.

In some embodiments, the Bacillus strain composition further comprises a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In some embodiments, the Bacillus strain composition comprises said at least one of Bacillus subtilis 747, Bacillus subtilis 839, Bacillus subtilis 1781, Bacillus subtilis 1999, Bacillus subtilis 2018, or an active variant thereof and said Bacillus subtilis 750 or active variant thereof in equal proportions.

In some embodiments, the Bacillus strain composition further comprises a bacterial cell, a spore, a forespore, and/or a combination of cells, spores, and/or forespores of said Bacillus subtilis 747 or active variant thereof. In some embodiments, the Bacillus strain composition comprises said Bacillus subtilis 747 or active variant thereof and said Bacillus subtilis 750 or active variant thereof in equal proportions.

In some embodiments, said bacterial cell, spore, forespore, and/or combination of cells, spores, and/or forespores of said Bacillus strain(s) is present in the composition at about 1×10⁵ CFU/gram to about 1×10¹⁰ CFU/gram or at about 1×10⁵ CFU/ml to about 1×10¹⁰ CFU/ml.

In some embodiments, said bacterial cell, spore, forespore, and/or combination of cells, spores, and/or forespores of said Bacillus subtilis 747 or active variant thereof and said Bacillus subtilis 750 or active variant thereof is present in the composition at about 1×10⁵ CFU/gram to about 1×10¹⁰ CFU/gram or at about 1×10⁵ CFU/ml to about 1×10¹⁰ CFU/ml.

In some embodiments, the Bacillus strain composition further comprises feed, forage, or fodder comprising said one or more polysaccharides or said one or more proteins. In some embodiments, said bacterial cell, spore, forespore, and/or combination of cells, spores, and/or forespores of said Bacillus strain(s) is present in the composition at about 1.5×10⁵ CFU/gram of feed, forage, or fodder to about 1.5×10⁶ CFU/gram of feed, forage, or fodder. In some embodiments, said bacterial cell, spore, forespore, and/or combination of cells, spores, and/or forespores of said Bacillus subtilis 747 or active variant thereof and said Bacillus subtilis 750 or active variant thereof is present in the composition at about 1.5×10⁵ CFU/gram of feed, forage, or fodder to about 1.5×10⁶ CFU/gram of feed, forage, or fodder.

In some embodiments, an effective amount of said Bacillus strain composition increases conversion of (i) one or more polysaccharides into short chain polysaccharides and/or monosaccharides and/or (ii) one or more proteins into short chain polypeptides and/or amino acids relative to the absence of said effective amount of said Bacillus strain composition.

In some embodiments, said one or more polysaccharides comprise one or more non-starch polysaccharides. In some embodiments, said one or more non-starch polysaccharides comprise arabinoxylan and/or cellulose. In some embodiments, said one or more proteins comprise zein and/or soy protein. In some embodiments, said Bacillus strain composition comprises an activity of arabinoxylanase, cellulase, amylase, zein protease, and/or soy protease.

In some embodiments, said Bacillus strain composition is formulated as pellets, mash, crumble, cake, meal, powder, or liquid.

In some embodiments, said Bacillus subtilis 750 is deposited under NRRL accession number B-68212. In some embodiments, said Bacillus subtilis 747 is deposited under NRRL accession number B-67257, said Bacillus subtilis 839 is deposited under NRRL accession number B-67951, said Bacillus subtilis 1781 is deposited under NRRL accession number B-67259, said Bacillus subtilis 1999 is deposited under NRRL accession number B-67318, and/or said Bacillus subtilis 2018 is deposited under NRRL accession number B-67261.

In one aspect, the present disclosure provides a method of increasing production of short chain polysaccharides, monosaccharides, short chain polypeptides, and/or amino acids, said method comprising contacting one or more polysaccharides and/or one or more proteins with an effective amount of a Bacillus strain composition comprising a bacterial cell, a spore, a forespore, and/or a combination of cells, spores, and/or forespores of Bacillus subtilis 750 or an active variant thereof.

In some embodiments, the Bacillus strain composition further comprises a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In some embodiments, the Bacillus strain composition comprises said at least one of Bacillus subtilis 747, Bacillus subtilis 839, Bacillus subtilis 1781, Bacillus subtilis 1999, Bacillus subtilis 2018, or an active variant thereof and said Bacillus subtilis 750 or active variant thereof in equal proportions.

In some embodiments, the Bacillus strain composition further comprises a bacterial cell, a spore, a forespore, and/or a combination of cells, spores, and/or forespores of said Bacillus subtilis 747 or active variant thereof. In some embodiments, the Bacillus strain composition comprises said Bacillus subtilis 747 or active variant thereof and said Bacillus subtilis 750 or active variant thereof in equal proportions.

In some embodiments, the method comprises contacting feed, forage, or fodder comprising said one or more polysaccharides and/or said one or more proteins with said effective amount of said Bacillus strain composition.

In one aspect, provided herein is a method of increasing production of short chain polysaccharides, monosaccharides, short chain polypeptides, and/or amino acids in the gastrointestinal tract of an animal, said method comprising feeding said animal an effective amount of a Bacillus strain composition comprising a bacterial cell, a spore, a forespore, and/or a combination of cells, spores, and/or forespores of Bacillus subtilis 750 or an active variant thereof.

In some embodiments, the Bacillus strain composition further comprises a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In some embodiments, the Bacillus strain composition comprises said at least one of Bacillus subtilis 747, Bacillus subtilis 839, Bacillus subtilis 1781, Bacillus subtilis 1999, Bacillus subtilis 2018, or an active variant thereof and said Bacillus subtilis 750 or active variant thereof in equal proportions.

In some embodiments, the Bacillus strain composition comprises a bacterial cell, a spore, a forespore, and/or a combination of cells, spores, and/or forespores of said Bacillus subtilis 747 or active variant thereof. In some embodiments, the Bacillus strain composition comprises said Bacillus subtilis 747 or active variant thereof and said Bacillus subtilis 750 or active variant thereof in equal proportions.

In some embodiments, said method increases digestibility of one or more polysaccharides and/or one or more proteins in the animal.

In some embodiments, said animal is fed one or more polysaccharides and/or one or more proteins. In some embodiments, said animal is fed feed, forage, or fodder comprising said one or more polysaccharides and/or said one or more proteins. In some embodiments, said Bacillus strain composition further comprises said one or more polysaccharides and/or said one or more proteins. In some embodiments, said Bacillus strain composition comprises feed, forage, or fodder comprising said one or more polysaccharides and/or said one or more proteins.

In some embodiments, said bacterial cell, spore, forespore, and/or combination of cells, spores, and/or forespores of said Bacillus strain(s) is present in said Bacillus strain composition at about 1.5×10⁵ CFU/gram of feed, forage, or fodder to about 1.5×10⁶ CFU/gram of feed, forage, or fodder. In some embodiments, said bacterial cell, spore, forespore, and/or combination of cells, spores, and/or forespores of said Bacillus subtilis 747 or active variant thereof and said Bacillus subtilis 750 or active variant thereof is present in said Bacillus strain composition at about 1.5×10⁵ CFU/gram of feed, forage, or fodder to about 1.5×10⁶ CFU/gram of feed, forage, or fodder.

In some embodiments, said method:
reduces apparent metabolizable energy (AME) required to maintain performance and/or body weight of the animal;
increases nutrition efficiency in the animal;
increases absorption of nutrients in the gastrointestinal tract of the animal;
reduces availability of nutrients in the hindgut of the animal for fermentation;
increases integrity of a gastrointestinal barrier in the animal;
increases an level of secretory IgA in the animal;
reduces inflammation in the animal;
reduces a level of alpha-1 acid glycoprotein (α-1-AGP) and/or IL-6 in the animal; and/or
inhibits a pathogen in the gastrointestinal tract of the animal; and/or
improves energy partitioning in the animal.

In some embodiments, the pathogen is one or more of Escherichia coli, Clostridium, Salmonella, and Streptococcus.

In some embodiments, said animal is an avian animal or a swine.

In some embodiments, said animal is a chicken, and the method reduces the amount of apparent metabolizable energy required to maintain performance and/or body weight by about 50 kcal per kg diet fed ad libitum to chickens.

In some embodiments, said effective amount comprises at about 1×10⁵ CFU/gram to about 1×10¹⁰ CFU/gram or at about 1×10⁵ CFU/ml to about 1×10¹⁰ CFU/ml of said bacterial cell, spore, forespore, and/or combination of cells, spores, and/or forespores of said Bacillus strain(s). In some embodiments, said effective amount comprises at about 1.5×10⁵ CFU/gram of feed, forage, or fodder to about 1.5×10⁶ CFU/gram of feed, forage, or fodder of said bacterial cell, spore, forespore, and/or combination of cells, spores, and/or forespores of said Bacillus strain(s).

In some embodiments, said effective amount comprises at about 1×10⁵ CFU/gram to about 1×10¹⁰ CFU/gram or at about 1×10⁵ CFU/ml to about 1×10¹⁰ CFU/ml of said bacterial cell, spore, forespore, and/or combination of cells, spores, and/or forespores of said Bacillus subtilis 747 or active variant thereof and said Bacillus subtilis 750 or active variant thereof. In some embodiments, said effective amount comprises at about 1.5×10⁵ CFU/gram of feed, forage, or fodder to about 1.5×10⁶ CFU/gram of feed, forage, or fodder of said bacterial cell, spore, forespore, and/or combination of cells, spores, and/or forespores of said Bacillus subtilis 747 or active variant thereof and said Bacillus subtilis 750 or active variant thereof.

In some embodiments, at least one of said Bacillus strains is a powdered, lyophilized strain. In some embodiments, said one or more polysaccharides comprise one or more non-starch polysaccharides. In some embodiments, said one or more non-starch polysaccharides comprise arabinoxylan and/or cellulose. In some embodiments, said Bacillus strain composition comprises an activity of arabinoxylanase, cellulase, amylase, zein protease, and/or soy protease. In some embodiments, said Bacillus strain composition is formulated as pellets, mash, crumble, cake, meal, powder, or liquid.

In some embodiments, said Bacillus subtilis 750 is deposited under NRRL accession number B-68212. In some embodiments, said Bacillus subtilis 747 is deposited under NRRL accession number B-67257, said Bacillus subtilis 839 is deposited under NRRL accession number B-67951, said Bacillus subtilis 1781 is deposited under NRRL accession number B-67259, said Bacillus subtilis 1999 is deposited under NRRL accession number B-67318, and/or said Bacillus subtilis 2018 is deposited under NRRL accession number B-67261.

### BRIEF DESCRIPTION OF THE DRAWINGS

FIG. 1 depicts transepithelial electrical resistance (TEER) in Caco2 cells without treatment, or a 24 hour treatment with TNF-α or the Bacillus subtilis 747 composition. Data are the mean+SD from four individual experiments (** p<0.01).

FIGS. 2A-2D depict intestinal functional markers (% change over positive control normalized with β-actin) in birds fed normal energy or −50 kcal reduced energy diet with or without supplementation with the Bacillus strain composition. “PC” and “NC” refer to normal energy diet and −50 kcal diet, respectively. “+DFM” refers to supplementation with the Bacillus strain composition. FIG. 2A depicts sodium glucose cotransport protein 1 (SGLT-1) levels in the four treatment groups. FIG. 2B depicts zona occludens-1 (ZO-1) levels in the four treatment groups. FIG. 2C depicts interferon-γ levels in the four treatment groups. FIG. 2D depicts IL-6 levels in the four treatment groups.

### DETAILED DESCRIPTION

The present disclosure now will be described more fully hereinafter. The disclosure may be embodied in many different forms and should not be construed as limited to the embodiments set forth herein; rather, these embodiments are provided so that this disclosure will satisfy applicable legal requirements.

### I. Overview

The prolonged use of antibiotic growth promoters can cause the development of resistance in pathogenic bacteria, which leads to a serious health risk for animals and humans. The beneficial effects of alternative products to antibiotics, e.g., prebiotics or probiotics, can be direct and indirect, and include the improvements in digestive physiology such as increases in the activity of digestive enzymes, thinning of the absorptive epithelium and increases in the digestibility of nutrients; other effects include modulation of the immune response at the level of the intestinal mucosa and at the systemic level, producing an adjuvant-like effect. Another outstanding mechanism of action of some prebiotics is the modulation of the intestinal microflora, stimulating the proliferation of beneficial bacteria and inhibiting the development of potentially pathogenic bacteria.

Provided herein are Bacillus strains, Bacillus strain compositions and methods for converting (i) one or more polysaccharides into short chain polysaccharides and/or monosaccharides and/or (ii) one or more proteins into short chain polypeptides and/or amino acids. The method provided herein can comprise contacting one or more polysaccharides and/or one or more proteins with an effective amount of the Bacillus strain composition provided herein, or feeding an animal with an effective amount of the Bacillus strain composition provided herein. Food source for domesticated animals, such as feed, forage, or fodder, commonly include certain polysaccharides (e.g., arabinoxylan, cellulose, starch) and/or proteins (e.g., zein, soy protein) that are difficult to digest or cannot be digested by the animals. An “domesticated animal” as used herein refers to animals that have been selectively bred and genetically adapted over generations to live alongside humans, e.g., to be kept and raised on a farm or ranch. Domesticated animals are genetically and behaviorally distinct from their naturally-existing counterparts in the wild. Domesticated animals include, but are not limited to, poultry (e.g., chickens, broilers, ducks, geese), pigs, cows, water buffalos, sheep, goats, turkeys, horses, donkeys, antelope, deer, elk, camel, llama, aplaca, rabbit, guinea pigs, rodents (e.g., mice, rats, ferret), dogs, cats, monkeys, apes, and insects (e.g., bees).

The present disclosure provides Bacillus subtilis 750 or an active variant thereof, which has enzymatic activities to break down certain polysaccharides and/or proteins commonly present in the food source for domesticated animals. For example, Bacillus subtilis 750 can have activities of at least arabinoxylanase, cellulase, amylase, zein protease, soy protease, and can break down polysaccharides and/or proteins, including arabinoxylan, hemicellulose, cellulose, starch, zein, and soy protein. Exogenous enzymes can be added to animal feed to improve digestibility of resistant fibers (Stefanello, C. et al. 2015 Poultry Science 94 (10): 2472-79). However, there are limitations in this approach because the exogenously added enzymes need to be able to remain active after exposure to high temperatures in the feed production (pelleting) and after exposure to strong acids as they transit the host's stomach. Bacillus subtilis 750 can be advantageous over enzymes exogenously added to feed, because Bacillus subtilis 750 can be stable during the feed production and transit to the stomach e.g., in spore form, and can exert enzymatic activity in the gastrointestinal tract of the animal. The present disclosure further provides Bacillus subtilis 747, Bacillus subtilis 839, Bacillus subtilis 1781, Bacillus subtilis 1999, Bacillus subtilis 2018, and/or an active variant thereof which can reduce pathogens (e.g., Escherichia coli, Clostridium, Salmonella, Streptococcus) and improve gut integrity (e.g., improve tight junction function of the intestinal endothelial cells) and immunity in animals. See, e.g., U.S. Pat. No. 10,201,574, WO 2017/205645, WO 2016/060934, each of which is incorporated by reference in its entirety. Bacillus subtilis 747, Bacillus subtilis 839, Bacillus subtilis 1781, Bacillus subtilis 1999, Bacillus subtilis 2018, and/or an active variant thereof can be stable during the feed production and transit to the stomach, e.g., in spore form, and can exert pathogen inhibitory activity in the gastrointestinal tract of the animal. Accordingly, provided herein are Bacillus strain compositions comprising a bacterial cell, a spore, a forespore, and/or a combination of cells, spores, and/or forespores of Bacillus strain Bacillus subtilis 750 or an active variant thereof. The Bacillus strain composition can further comprise a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In specific embodiments, the Bacillus strain composition comprises a bacterial cell, a spore, a forespore, and/or a combination of cells, spores, and/or forespores of Bacillus strain Bacillus subtilis 750 or an active variant thereof and Bacillus strain Bacillus subtilis 747 or an active variant thereof.

The Bacillus strains provided herein were isolated under the selective pressure of a commercial farm in which the animals are kept in a man-made environment, largely fed a man-made, rationed diet, exposed to the pathogens and stresses resulting from the conditions of the non-natural environment, and administered non-natural interventions such as medications. Thus, there is no evidence that the Bacillus strains of the present invention, i.e., Bacillus subtilis 750, Bacillus subtilis 747, Bacillus subtilis 839, Bacillus subtilis 1781, Bacillus subtilis 1999, Bacillus subtilis 2018, and their active variants, having enzymatic activities to break down polysaccharides or proteins, pathogen inhibitory activities, and/or activities to enhance gut integrity and immunity exist outside of a man-made environment, where there are less or no exposure to the selective pressure of stresses and pathogens resulting from the conditions of that artificial environment.

The Bacillus strains provided herein, and the compositions and methods comprising the Bacillus strains provided herein allow for the use of a probiotic that is capable of surviving in the gastrointestinal tract to produce short chain polysaccharides, monosaccharides, short chain polypeptides, and/or amino acids in the intestine by biotransforming polysaccharides (e.g., arabinoxylan, cellulose, starch) and/or proteins (e.g., zein, soy) that are commonly present in the diet of domesticated animals, inhibit pathogens, and/or enhance gut integrity and immunity. The Bacillus strains provided herein and the compositions and methods comprising the Bacillus strains provided herein can increase digestibility of one or more polysaccharides and/or one or more proteins in the animal diet. The short chain polysaccharides, monosaccharides, short chain polypeptides, and/or amino acids produced by the compositions or methods provided herein comprising Bacillus strains can then be further digested and/or absorbed into the body after production. As used herein, “digestible” nutrients (e.g., polysaccharide, protein) or energy refers to the gross nutrients or energy of the food consumed minus the gross energy contained in the feces, with appropriate corrections for nitrogen excretion. “Metabolizable” nutrients or energy, also referred to as “apparent metabolizable energy” (AME), as used herein refers to the gross nutrients or energy of the food consumed minus the gross energy contained the feces, urine, gaseous products of the digestion, and the remaining digesta on a specified segment of the gastro-intestinal tract of the animal, e.g. the ileum, with appropriate corrections for nitrogen excretion. “Net” nutrients or energy as used herein refers to the gross nutrients or energy of the food consumed minus the gross energy contained the feces, urine, gaseous products of the digestion, the remaining digesta on a specified segment of the gastro-intestinal tract of the animal, and nutrients or energy lost by heat, with appropriate corrections for nitrogen excretion. Energy digestibility, nutrient digestibility, metabolizable energy, metabolizable nutrients, net energy, or net nutrients can be measured by means of the total collection of excreta (e.g., feces, urine, gaseous products) during a period of time; or with the use of an inert marker that is not absorbed by the animal. Such an inert marker may be titanium dioxide, chromic oxide or acid insoluble ash. In the methods using markers, digestibility of nutrients can be calculated for instance according to the formula: digestibility (%)=1−(% marker in feed/% marker in feces)×(% nutrient feces/% nutrient feed). Digestibility can be expressed as a percentage of the nutrient in the feed, or as mass units of digestible nutrient per mass units of nutrient in the feed. As used herein, “nitrogen retention” means a subject's ability to retain nitrogen from the diet as body mass. A negative nitrogen balance occurs when the excretion of nitrogen exceeds the daily intake and is often seen when the muscle is being lost. A positive nitrogen balance is often associated with muscle growth, particularly in growing animals. Nitrogen retention may be measured as the difference between the intake of nitrogen and the excreted nitrogen by means of the total collection of excreta and urine during a period of time. It is understood that excreted nitrogen includes undigested protein from the feed, endogenous proteinaceous secretions, microbial protein, and urinary nitrogen.

The Bacillus strains and compositions described herein, in some embodiments with one or more polysaccharides and/or one or more proteins, or feed, forage, or fodder comprising the one or more polysaccharides and/or one or more proteins, can be formulated as pellets, mash, crumble, cake, meal, powder, liquid, capsule, or other formulations and administered orally as a direct fed microbial composition or in feed, forage, or fodder, or via other administration routes.

As used herein, a “polysaccharide” refers to a long chain of carbohydrate molecule, comprising a plurality of monosaccharides linked by glycosidic bonds. A polysaccharide can contain more than 3, 5, 10, 20, 30, 40, 50, 60, 70, 80, 90, 100, or more than 100 monosaccharide units. Exemplary polysaccharides include starch, glycogen, cellulose, and arabinoxylan. As used herein, a “short chain polysaccharide” refers to a polysaccharide or an oligosaccharide containing a plurality of monosaccharides, less than those present in a “polysaccharide”, linked by glycosidic bonds. An oligosaccharide commonly contain about 3-5 monosaccharides. Depending on how many monosaccharide units a specific polysaccharide (e.g., substrate polysaccharide) contains, a short chain polysaccharide can contain any number of monosaccharide units that are less than that in the specific polysaccharide. As used herein, a “monosaccharide” refers to a carbohydrate unit that cannot be hydrolyzed to smaller carbohydrates. A monosaccharide comprises aldehydes or ketones possessing two or more hydroxyl groups. Exemplary monosaccharides include glucose, fructose, galactose, and ribose.

As used herein, “arabinoxylanase” refers to an enzyme that catalyzes the hydrolysis of α- or β-xylosidic linkages. Arabinoxylanase can hydrolyze arabinoxylan. As used herein, “arabinoxylan” refers to a hemicellulose containing copolymers of two pentose sugars, arabinose and xylose. Arabinoxylan is found in the primary and secondary cell walls of plants. As used herein, “amylase” refers to an enzyme that catalyzes the hydrolysis of starch into sugars. As used herein, “starch” refers to a polysaccharide containing glucose units linked by glycosidic bonds. As used herein, “cellulase” refers to an enzyme that catalyzes cellulolysis, the decomposition of cellulose and of some related polysaccharides. Several different kinds of cellulases are known, which differ structurally and mechanistically. Cellulases can include endo-1,4-β-D-glucanase (β-1,4-glucanase, β-1,4-endoglucan hydrolase, endoglucanase D, 1,4-(1,3;1,4)-β-D-glucan 4-glucanohydrolase), carboxymethyl cellulase (CMCase), avicelase, celludextrinase, cellulase A, cellulosin AP, alkali cellulase, cellulase A 3, β 1,4 glucan-4-glucanohydrolase (9.5 cellulase), and pancreatin. As used herein, “cellulose” refers to a polysaccharide comprising a linear chain of β-1,4 linked d-glucose units. As used herein, a “short chain polypeptide” refers a polypeptide containing a plurality of amino acids, less than those present in a “protein”, linked by peptide bonds. Depending on how many amino acid units a specific protein (e.g., substrate protein) contains, a short chain polypeptide can contain any number of amino acid units that are less than that in the specific protein. As used herein, “zein” refers to a class of prolamine protein comprising a high amount of hydrophobic amino acids (e.g., proline glutamine, asparagine). Zein can be found in plants, e.g., maize. As used herein, “soy protein” refers to a protein present in or isolated from soybeans. Zein protease catalyzes proteolysis of zein into short chain polypeptides and/or amino acids. Soy protein can be made from dehulled and defatted soybeans. Soy protease catalyzes proteolysis of soy protein into short chain polypeptides and/or amino acids.

The Bacillus strains, the Bacillus strains compositions, and the methods comprising the Bacillus strains provided herein, can increase nutrition efficiency in an animal. “Nutrition” as used herein refers to elements in food (e.g., feed, forage, fodder) that are used by the animal for growth and production. Nutrition can include water, protein, carbohydrates, minerals, and vitamins. “Feed efficiency”, “nutrition utilization efficiency”, “nutrition efficiency”, “energy efficiency”, or “food efficiency” as used herein refers to the amount of weight gain in an animal that occurs when the animal is fed ad-libitum or a specified amount of food during a period of time. As used herein, the term “feed conversion ratio”, “energy conversion ratio”, “nutrition conversion ratio”, or “food conversion ratio” refers to the amount of feed, energy, nutrition, or food fed to an animal to increase the weight of the animal by a specified amount. By “improved (or increased, high) nutrition (or energy, food, or feed) efficiency” or “improved (or decreased, low) feed (or energy, nutrition, food) conversion ratio” is meant that the use of the Bacillus strain composition in feed results in a lower amount of feed (or energy, nutrition, food) being required to be fed to an animal to maintain the performance of the animal, or to increase the weight of the animal by a specified amount, relative to the amount of feed required to achieve the same body weight and/or performance when the animal is not fed with the Bacillus strain composition. As used herein, “performance” of an animal can be determined by the feed efficiency of an animal, weight gain of the animal, feed conversion ratio, digestibility of a nutrient in a feed (e.g. polysaccharide digestibility, amino acid digestibility), digestible energy or metabolizable energy in a feed, nitrogen retention, animals' ability to avoid the negative effects of pathogens, and/or immune response of the animal. Animal performance can be measured by one or more of the following parameters: average daily gain (ADG), weight, number of eggs produced, scours, mortality, feed conversion (both feed: gain and gain: feed), and feed intake. For example, performance of poultry can be assessed by feed conversion ratio, which can be calculated as total amount of food consumed divided by amount of weight gained or number of eggs produced.

Without wishing to be bound by any theory, the Bacillus strains provided herein, or the compositions and methods comprising the Bacillus strains provided herein, can improve nutrition efficiency and reduce an amount of apparent metabolizable energy (AME) required to maintain performance and/or body weight of the animal via one or more mechanisms, including but not limited to increasing absorption of nutrients in the gastrointestinal tract of the animal; reducing availability of nutrients in the hindgut of the animal for fermentation; increasing integrity of a gastrointestinal barrier (gut barrier) in the animal; enhancing gastrointestinal immunity in the animal, including increasing mucosal immunity (e.g., increasing levels of secretory IgA) and reducing inflammation (e.g., reducing a level of α-1-AGP and/or IL-6 in the animal); inhibiting a pathogen (e.g., Escherichia coli, Clostridium, Salmonella, and Streptococcus) in the gastrointestinal tract of the animal; and/or improving nutrient partitioning (or energy partitioning). “Nutrient partitioning” or “energy partitioning” as used herein refers to a process by which the nutrients or energy is used for synthesis (e.g., protein synthesis, body weight gain, growth) or consumptive needs (e.g., building or maintaining immunity, defending against pathogens, metabolizing nutrients). Improved or efficient nutrient partitioning (or energy partitioning) as used herein refers to an increased use of nutrients or energy for synthesis (e.g., protein synthesis, body weight gain, growth), which can be assessed by increased body weight or performance in animals fed diet having a specified amount of energy (e.g., AME) as compared to a control animal fed the same diet.

The presently disclosed bacterial strains can be used as probiotics. The term “probiotics” has been defined by the Food and Agriculture Organization of the United Nations (FAO) and World Health Organization (WHO) as live microorganisms which when administered in adequate amounts confer a health benefit on the host. Probiotics include beneficial bacteria that when consumed or otherwise administered to the gastrointestinal tract improve the health of the subject. In some embodiments, the beneficial bacteria colonize the gut, allowing for a more persistent beneficial effect.

### II. Definitions

Like numbers refer to like elements throughout. As used in this specification and the claims, the singular forms “a,” “an,” and “the” include plural references unless the context clearly dictates otherwise.

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, melt index, temperature etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

As used herein, “animal” includes but is not limited to human, mammal, amphibian, bird, reptile, pigs, cows, cattle, goats, horses, sheep, poultry, and other animals kept or raised on a farm or ranch, sheep, big-horn sheep, buffalo, antelope, oxen, donkey, mule, deer, elk, caribou, water buffalo, camel, llama, alpaca, rabbit, mouse, rat, guinea pig, hamster, ferret, dog, cat, and other pets, primate, monkey, ape, and gorilla. In some embodiments, the animals are avian animals, including but not limited to chicken and broilers.

As used herein, “livestock” refers to any farmed animal. In one embodiment, livestock is one or more of poultry such as chickens, broilers, ducks, geese, turkeys; ruminants such as cattle (e.g. cows, bulls, calves); pigs (including piglets); birds; aquatic animals such as fish, agastric fish, gastric fish, freshwater fish such as salmon, cod, trout and carp, marine fish such as sea bass, and crustaceans such as shrimps, mussels and scallops; horses (including race horses); and sheep (including lambs).

By “at least one strain” is meant a single strain but also mixtures of strains comprising at least two strains of bacteria. By “a mixture of at least two strains,” is meant a mixture of two, three, four, five, six or more strains. In some embodiments of a mixture of strains, the proportions can vary from 1% to 99%. In certain embodiments, the proportion of a strain used in the mixture is at least 5%, 10%, 15%, 20%, 25%, 30%, 35%, 40%, 45%, 50%, 55%, 60%, 65%, 70%, 75%, 80%, 85%, 90%, or 95%. Other embodiments of a mixture of strains are from 25% to 75%. Additional embodiments of a mixture of strains are approximately 50% for each strain. When a mixture comprises more than two strains, the strains can be present in equal or substantially equal proportions in the mixture or in different proportions. “Equal” proportions or amounts as used herein in the context of two or more strains refer to the presence of each strain in similar or substantially same proportions. For example, “equal” proportions or amounts include the presence of two or more strains at proportions or in amounts that are within 0, 1, 2, 3, 4, 5, 6, 7, 8, 9, 10, 11, 12, 13, 14, 15, 16, 17, 18, 19, or 20% difference between each other (or among one another).

By “administer” or “administering” is meant the action of introducing the strain or a composition to a subject or an environment.

As used herein, to “contact,” “contacting,” or “contacted” refers to the indirect or direct application of the Bacillus strains or Bacillus strain composition disclosed herein to a product, including but not limited to one or more polysaccharides, one or more proteins, or feed, forage, or fodder comprising the one or more polysaccharides or proteins. Examples of the application methods which may be used, include, but are not limited to, direct application by mixing the Bacillus strains or Bacillus strain composition with the product, spraying the Bacillus strains or Bacillus strain composition onto the product surface, or dipping the product into a liquid preparation of the Bacillus strains or Bacillus strain composition.

As used herein, “effective amount” is meant a quantity of strain, and/or the combination of strains thereof to produce desired effect, e.g., converting one or more polysaccharides and/or proteins into short chain polysaccharides, monosaccharides, short chain polypeptides, and/or amino acids; increasing digestibility of one or more polysaccharides and/or proteins; reducing an amount of apparent metabolizable energy (AMIE) required to maintain performance and/or body weight of the animal; increasing nutrition efficiency in the animal; increasing absorption of nutrients in the gastrointestinal tract of the animal; reducing availability of nutrients in the hindgut of the animal for fermentation; increasing integrity of a gastrointestinal barrier (gut barrier) in the animal; increasing an level of secretory IgA in the animal; reducing inflammation in the animal; reducing the level of α-1-AGP and/or IL-6 in the animal; inhibiting a pathogen in the gastrointestinal tract of the animal; and/or improving performance of an animal. Improvement in performance can be measured as described herein or by other methods known in the art. An effective amount can be administered to the animal by providing ad libitum access to feed, forage, or fodder containing the Bacillus strain composition. The Bacillus strain composition can also be administered (e.g., fed) in one or more doses.

As used herein, the term “compound feed” refers to a commercial feed in the form of a meal, a pellet, a mash, nuts, cake, a crumble, and the like. Compound feeds may be blended from various raw materials and additives. These blends are formulated according to the specific requirements of the target animal.

As used herein, “immune response” refers to one of the multiple ways in which the Bacillus strain composition provided herein modulate the immune system of animals, including increased antibody production (e.g., IgA, IgG), up-regulation of cell mediated immunity, increase or decrease of inflammatory cytokines (e.g., reduction in IL-6 levels), increase or decrease of acute phase protein (e.g., α-1-AGP) and augmented toll-like receptor signaling. Without wishing to be bound by theory, immuno-stimulation of the gastrointestinal tract by Bacillus strain composition provided herein can be advantageous to protect the host against disease, and immuno-suppression of the gastrointestinal tract may be advantageous to the host because less nutrients and energy are used to support the immune function.

As used herein, “microbial” refers to microorganism. A “pathogen” refers to a pathogenic microorganism, e.g., a microorganism that causes disease or decrease in performance in an animal. As used herein, “reducing the growth of microorganism (or pathogen)” includes but is not limited to reducing the growth of microorganisms or pathogens, e.g., by at least 1%, 5%, 10%, 20%, 30%, 40%, 50%, by about 1%, 5%, 10%, 20%, 30%, 40%, 50%, 60%, 70%, 80%, 90%, more than 90%, by about 1-5%, 5-10%, 10-20%, 20-30%, 30-40%, 40-50%, 50-60%, 60-70%, 70-80%, 80-90%, 90-99%, or 100%.

As used herein with respect to a parameter, the term “increased” or “increasing” or “increase” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100%, 120%, 150%, 200%, 300%, 400%, 500%, or more) positive change in the parameter from a comparison control, e.g., an established normal or reference level of the parameter, or an established standard control. Accordingly, the terms “increased”, “increase”, and the like encompass both a partial increase and a significant increase compared to a control.

As used herein with respect to a parameter, the term “decreased” or “decreasing” or “decrease” or “reduced” or “reducing” or “reduce” or “lower” or “loss” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “decreased”, “reduced”, and the like encompass both a partial reduction and a complete reduction compared to a control.

### III. Composition Comprising a Bacterial Strain

### A. Bacterial Strains

Bacterial strains are provided which can (i) convert one or more polysaccharides into short chain polysaccharides and/or monosaccharides and/or one or more proteins into short chain polypeptides and/or amino acids, and/or (ii) inhibit growth of one or more pathogens. Such bacterial strains include Bacillus subtilis 750 or an active variant thereof, which can convert one or more polysaccharides into short chain polysaccharides and/or monosaccharides and/or one or more proteins into short chain polypeptides and/or amino acids or an active variant thereof. Such bacterial strains can also include Bacillus subtilis 747, Bacillus subtilis 839, Bacillus subtilis 1781, Bacillus subtilis 1999, Bacillus subtilis 2018, and/or an active variant thereof, which can inhibit growth of one or more pathogens. Cell populations comprising Bacillus subtilis strain 750 and/or an active variant thereof are provided, as well as populations of spores derived from each of these strains, or any preparation thereof. In further embodiments, cell populations comprising Bacillus strains (i) Bacillus subtilis 750 or an active variant thereof and (ii) at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof are provided, as well as populations of spores derived from each of these strains, or any preparation thereof. Thus, bacterial strains or bacterial strain compositions provided herein comprise as an active ingredient a cell population, spore, forespore, or combination thereof, of Bacillus subtilis strain 750, Bacillus subtilis strain 747, Bacillus subtilis 839, Bacillus subtilis 1781, Bacillus subtilis 1999, Bacillus subtilis 2018, and/or an active variant thereof. In specific embodiments, bacterial strains or bacterial strain compositions provided herein comprise a bacterial cell, a spore, a forespore, and/or a combination of cells, spores, and/or forespores of Bacillus strain Bacillus subtilis 750 or an active variant thereof. In other embodiments, bacterial strains or bacterial strain compositions provided herein comprise a bacterial cell, a spore, a forespore, and/or a combination of cells, spores, and/or forespores of (i) Bacillus strain Bacillus subtilis 750 or an active variant thereof and (ii) at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In specific embodiments, bacterial strains or bacterial strain composition comprise a bacterial cell, a spore, a forespore, and/or a combination of cells, spores, and/or forespores of Bacillus subtilis 747 or an active variant thereof, and a bacterial cell, a spore, a forespore, and/or a combination of cells, spores, and/or forespores of Bacillus subtilis 750 or an active variant thereof.

Bacillus subtilis strains 747, 1781, 1999, and 2018 were deposited with the Patent Depository of the National Center for Agricultural Utilization Research Agricultural Research Service, U.S. Department of Agriculture, 1815 North University Street, Peoria, Illinois 61604 U.S.A. on May 24, 2016. Bacillus subtilis strain 747 was assigned NRRL No. B-67257. Bacillus subtilis strain 1781 was assigned NRRL No. B-67259. Bacillus subtilis strain 1999 was assigned NRRL No. B-67318. Bacillus subtilis strain 2018 was and assigned NRRL No. B-67261.

Bacillus subtilis strain 839 was deposited with the Patent Depository of the National Center for Agricultural Utilization Research Agricultural Research Service, U.S. Department of Agriculture, 1815 North University Street, Peoria, Illinois 61604 U.S.A. on Apr. 12, 2020 and assigned NRRL No. B-67951.

Bacillus subtilis strain 750 was deposited with said Patent Depository of the National Center for Agricultural Utilization Research Agricultural Research Service, U.S. Department of Agriculture, 1815 North University Street, Peoria, Illinois 61604 U.S.A. on Oct. 26, 2022 and assigned NRRL No. B-68212.

Each of the deposits identified above will be maintained under the terms of the Budapest Treaty on the International Recognition of the Deposit of Microorganisms for the Purposes of Patent Procedure. Each deposit was made merely as a convenience for those of skill in the art and is not an admission that a deposit is required under 35 U.S.C. § 112.

The term “isolated” encompasses a bacterium, spore, or other entity or substance, that has been (1) separated from at least some of the components with which it was associated when initially produced (in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated. An “isolated” bacterial strain includes not only a biologically pure strain, but also any culture of organisms that is grown or maintained in a condition that is not found in nature.

As used herein, a substance is “pure” if it is substantially free of other components. The terms “purify,” “purifying” and “purified” refer to a bacterium, spore, or other material that has been separated from at least some of the components with which it was associated either when initially produced or generated (e.g., in nature or in an experimental setting), or during any time after its initial production. A bacterium or spore or a bacterial population or a spore population may be considered purified if it is isolated at or after production, such as from a material or environment containing the bacterium or bacterial population or spore, and a purified bacterium or bacterial population or spore may contain other materials up to about 10%, about 20%, about 30%, about 40%, about 50%, about 60%, about 70%, about 80%, about 90%, or above about 90% and still be considered purified. In some embodiments, purified bacteria or spores and bacterial populations or spore populations are more than about 80%, about 85%, about 90%, about 91%, about 92%, about 93%, about 94%, about 95%, about 96%, about 97%, about 98%, about 99%, or more than about 99% pure. In specific embodiments, a culture of bacteria contains no other bacterial species in quantities to be detected by normal bacteriological techniques.

By “population” is intended a group or collection that comprises two or more (i.e., 10, 100, 1,000, 10,000, 1×10⁶, 1×10⁷, or 1×10⁸ or greater) cells, spores, forespores, or combination thereof of a given bacterial strain. Various compositions are provided herein that comprise a population of at least one bacterial strain or a mixed population of individuals from more than one bacterial strain. A colony forming unit (CFU) is the viable cell count of a sample resulting from standard microbiological plating methods. The term is derived from the fact that a single cell when plated on appropriate medium will grow and become a viable colony in the agar medium. Since multiple cells may give rise to one visible colony, the term colony forming unit can be a more useful unit measurement than cell number. In specific embodiments, the population of Bacillus subtilis 747, Bacillus subtilis 750, an active variant thereof, or combination thereof comprises a concentration of at least about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁴ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁴ CFU/ml to about 10¹⁰ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, about 10⁹ CFU/ml to about 10¹⁰ CFU/ml, about 10⁵ CFU/ml to about 10⁹ CFU/ml, about 10⁵ CFU/ml to about 10⁸ CFU/ml, about 10⁵ CFU/ml to about 10⁷ CFU/ml, or about 10⁵ CFU/ml to about 10⁶ CFU/ml. In other embodiments, the concentration of the bacterial strain provided herein, an active variant thereof, or combination thereof comprises or consists of at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml. In specific embodiments, the population of Bacillus subtilis 747, Bacillus subtilis 750, an active variant thereof, or combination thereof comprises a concentration of at least about 10⁴ CFU/g to about 10¹⁰ CFU/g, 10⁴ CFU/g to about 10¹¹ CFU/g, 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 10⁵ CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, about 10⁹ CFU/g to about 10¹⁰ CFU/g, about 10⁵ CFU/g to about 10⁹ CFU/g, about 10⁵ CFU/g to about 10⁸ CFU/g, about 10⁵ CFU/g to about 10⁷ CFU/g, or about 10⁵ CFU/g to about 10⁶ CFU/g. In other embodiments, the concentration of the bacterial strain provided herein, active variant thereof, or combination thereof comprises or consists of at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g.

Populations or cultures of Bacillus subtilis 747, Bacillus subtilis 750, and/or active variants thereof can be produced by cultivation of the bacterial strain. Cultivation can be started by scaling-up a seed culture. This involves repeatedly and aseptically transferring the culture to a larger and larger volume to serve as the inoculum for the fermentation, which can be carried out in large stainless steel fermentors in medium containing proteins, carbohydrates, and minerals necessary for optimal growth of the strain. Non-limiting exemplary media is Tryptic Soy Broth (TSB) for the Bacillus strains. After the bacterial inoculum is added to the fermentation vessel, the temperature and agitation are controlled to allow maximum growth. Once the culture reaches a maximum population density, the culture is harvested by separating the cells from the fermentation medium. This separation is commonly performed by centrifugation. The concentration of the bacterial culture can be measured from any sample of fermentation broth or bacterial strain composition.

A “spore” or “endospore” refers to at least one dormant (at application) but viable reproductive unit of a bacterial species. Non-limiting methods by which spores are formed from each of Bacillus subtilis 747 and Bacillus subtilis 750 (or variants of any thereof) are disclosed elsewhere herein. It is further recognized the populations disclosed herein can comprise a combination of vegetative cells and “forespores” (also referred to as “prespores”; the smaller of the two compartments that are formed by asymmetric division of cells in an intermediate stage of spore formation); a combination of forespores and spores; or a combination of forespores, vegetative cells and/or spores. In specific embodiments, the Bacillus subtilis 747 or Bacillus subtilis 750 (or variant of any thereof) is a viable cell, spore, or forespore.

### B. Active Variants of a Bacterial Strain

Further provided are active variants of Bacillus subtilis 750, Bacillus subtilis 747, Bacillus subtilis 839, Bacillus subtilis 1781, Bacillus subtilis 1999, and/or Bacillus subtilis 2018. Active variants of the various bacterial strains provided herein include, for example, any isolate or mutant of Bacillus subtilis 750, Bacillus subtilis 747, Bacillus subtilis 839, Bacillus subtilis 1781, Bacillus subtilis 1999, and/or Bacillus subtilis 2018 that retains the ability to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus). An active variant of Bacillus subtilis 750, Bacillus subtilis 747, Bacillus subtilis 839, Bacillus subtilis 1781, Bacillus subtilis 1999, and/or Bacillus subtilis 2018 can, for example, retain at least 60% (e.g., 60%, 65%, 70%, 75%, 80%, 85%, 90%, 95%, 96%, 97%, 98%, 99%, or 100%) enzymatic activity to convert polysaccharides and/or proteins and/or pathogen inhibitory activity as compared to Bacillus subtilis 750, Bacillus subtilis 747, Bacillus subtilis 839, Bacillus subtilis 1781, Bacillus subtilis 1999, and/or Bacillus subtilis 2018. An active variant includes a strain having all of the identifying characteristics of the recited strain. A “strain of the invention” or a “strain provided herein” includes active variants thereof.

By “modified bacterial strain” is intended a population wherein the strain has been modified (by selection and/or transformation) to have one or more additional traits of interest. Modified bacterial strains can be made through genetic engineering techniques and such engineered or recombinant bacterial strains grown to produce a modified population of bacterial strains. A recombinant bacterial strain can be produced by introducing polynucleotides into the bacterial host cell by transformation or by otherwise altering the native bacterial chromosome sequence, including but not limited to, gene editing approaches. Methods for transforming microorganisms are known and available in the art. See, generally, Hanahan, D. (1983) Studies on transformation of Escherichia coli with plasmids J. Mol. Biol. 166, 557-77; Seidman, C. E. (1994) In: Current Protocols in Molecular Biology, Ausubel, F. M. et al. eds., John Wiley and Sons, N Y; Choi et al. (2006) J. Microbiol. Methods 64:391-397; Wang et al. 2010. J. Chem. Technol. Biotechnol. 85:775-778. Transformation may occur by natural uptake of naked DNA by competent cells from their environment in the laboratory. Alternatively, cells can be made competent by exposure to divalent cations under cold conditions, by electroporation, by exposure to polyethylene glycol, by treatment with fibrous nanoparticles, or other methods well known in the art.

Active variants of the various bacteria provided herein can be identified by employing, for example, methods that determine the sequence identity relatedness between the 16S ribosomal RNA, methods to identify groups of derived and functionally identical or nearly identical strains include Multi-locus sequence typing (MLST), concatenated shared genes trees, Whole Genome Alignment (WGA), Average Nucleotide Identity, and MinHash (Mash) distance metric.

In one aspect, the active variants of Bacillus subtilis 750, Bacillus subtilis 747, Bacillus subtilis 839, Bacillus subtilis 1781, Bacillus subtilis 1999, and/or Bacillus subtilis 2018 include strains that are closely related to any of the disclosed strains by employing the Bishop MLST method of organism classification as defined in Bishop et al. (2009) BMC Biology 7 (1) 1741-7007-7-3. Thus, in specific embodiments, an active variant of a bacterial strain disclosed herein includes a bacterial strain that falls within at least a 80%, 85%, 86%, 87%, 88%, 89%, 90%, 91%, 92%, 93%, 94%, 95%, 96%, 97%, 98%, 98.5%, 98.8%, 99%, 99.1%, 99.2%, 99.3%, 99.4%, 99.5%, 99.6%, 99.7%, 99.8%, or 99.9% sequence cut off employing the Bishop method of organism classification as set forth in Bishop et al. (2009) BMC Biology 7 (1) 1741-7007-7-3, which is herein incorporated by reference in its entirety. Active variants of the bacteria identified by such methods will retain the ability to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus).

In some embodiments, the active variant of the bacterial strain(s) disclosed herein include strains that are closely related to any of the disclosed strains on the basis of the Average Nucleotide Identity (ANI) method of organism classification. ANI (see, for example, Konstantinidis, K. T., et al., (2005) PNAS USA 102 (7): 2567-72; and Richter, M., et al., (2009) PNAS 106 (45): 19126-31) and variants (see, for example, Varghese, N.J., et al., Nucleic Acids Research (Jul. 6, 2015): gkv657) are based on summarizing the average nucleotides shared between the genomes of strains that align in WGAs. Thus, in specific embodiments, an active variant of bacterial strain Bacillus subtilis 750, Bacillus subtilis 747, Bacillus subtilis 839, Bacillus subtilis 1781, Bacillus subtilis 1999, and/or Bacillus subtilis 2018 disclosed herein includes a bacterial strain that falls within at least a 90%, 95%, 96%, 97%, 97.5%, 98%, 98.5%, 98.8%, 99%, 99.5%, or 99.8% sequence cut off employing the ANI method of organism classification as set forth in Konstantinidis, K. T., et al., (2005) PNAS USA 102 (7): 2567-72, which is herein incorporated by reference in its entirety. Active variants of the bacteria identified by such methods will retain the ability to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus). In particular embodiments, the active variants of the isolated bacterial strain(s) disclosed herein include strain(s) that are closely related to Bacillus subtilis 750, Bacillus subtilis 747, Bacillus subtilis 839, Bacillus subtilis 1781, Bacillus subtilis 1999, and/or Bacillus subtilis 2018 on the basis of 16S rDNA sequence identity. See Stackebrandt E, et al., “Report of the ad hoc committee for the re-evaluation of the species definition in bacteriology,” Int J Syst Evol Microbiol. 52 (3): 1043-7 (2002) regarding use of 16S rDNA sequence identity for determining relatedness in bacteria. In an embodiment, the active variant is at least 95% identical to any of the above strains on the basis of 16S rDNA sequence identity, at least 96% identical to any of the above strains on the basis of 16S rDNA sequence identity, at least 97% identical to any of the above strains on the basis of 16S rDNA sequence identity, at least 98% to any of the above strains on the basis of 16S rDNA sequence identity, at least 98.5% identical to any of the above strains on the basis of 16S rDNA sequence identity, at least 99% identical to any of the above strains on the basis of 16S rDNA sequence identity, at least 99.5% to any of the above strains on the basis of 16S rDNA sequence identity or at least 100% to any of the above strains on the basis of 16S rDNA sequence identity. Active variants of the bacteria identified by such methods will retain the ability to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus).

The MinHash (Mash) distance metric is a comparison method that defines thresholds for hierarchical classification of microorganisms at high resolution and requires few parameters and steps (Ondov et al. (2016) Genome Biology 17:132). The Mash distance estimates the mutation rate between two sequences directly from their MinHash sketches (Ondov et al. (2016) Genome Biology 17:132). Mash distance strongly corresponds to Average Nucleotide Identity method (ANI) for hierarchical classification (See, Konstantinidis, K. T. et al. (2005) PNAS USA 102 (7): 2567-72, herein incorporated by reference in its entirety). That is, an ANI of 97% is approximately equal to a Mash distance of 0.03, such that values put forth as useful classification thresholds in the ANI literature can be directly applied with the Mash distance.

Active variants of the bacterial strain(s) disclosed herein include strains that are closely related to Bacillus subtilis 750, Bacillus subtilis 747, Bacillus subtilis 839, Bacillus subtilis 1781, Bacillus subtilis 1999, and/or Bacillus subtilis 2018 on the basis of the Minhash (Mash) distance between complete genome DNA sequences. Thus, in specific embodiments, an active variant of a bacterial strain disclosed herein includes bacterial strains having a genome within a Mash distance of less than about 0.015 to the disclosed strains. In other embodiments, an active variant of a bacterial strain disclosed herein includes a distance metric of less than about 0.001, 0.0025, 0.005, 0.010, 0.015, 0.020, 0.025, or 0.030. A genome, as it relates to the Mash distance includes both bacterial chromosomal DNA and bacterial plasmid DNA. In other embodiments, the active variant of a bacterial strain has a genome that is above a Mash distance threshold to the disclosed strains that is greater than dissimilarity caused by technical variance. In further instances, the active variant of a bacterial strain has a genome that is above a Mash distance threshold to the disclosed strains that is greater than dissimilarity caused by technical variance and has a Mash distance of less than about 0.015. In other instances, the active variant of a bacterial strain has a genome that is above a Mash distance threshold to the disclosed strains that is greater than dissimilarity caused by technical variance and has a Mash distance of less than about 0.001, 0.0025, 0.005, 0.010, 0.015, 0.020, 0.025, or 0.030.

As used herein, “above technical variation” means above the Mash distance between two strains caused by errors in the genome assemblies provided the genomes being compared were each DNA sequenced with at least 20X coverage with the Illumina HiSeq 2500 DNA sequencing technology and the genomes are at least 99% complete with evidence for contamination of less than 2%. While 20X coverage is an art recognized term, for clarity, an example of 20X coverage is as follows: for a genome size of 5 megabases (MB), 100 MB of DNA sequencing from the given genome is required to have 20X sequencing coverage on average at each position along the genome. There are many suitable collections of marker genes to use for genome completeness calculations including the sets found in Campbell et al. (2013) PNAS USA 110 (14): 5540-45, Dupont et al. (2012) ISMEJ 6:1625-1628, and the CheckM framework (Parks et al. (2015) Genome Research 25:1043-1055); each of these references is herein incorporated in their entirety. Contamination is defined as the percentage of typically single copy marker genes that are found in multiple copies in the given genome sequence (e.g. Parks et al. (2015) Genome Research 25:1043-1055); each of these references is herein incorporated in their entirety. Completeness and contamination are calculated using the same collection of marker genes. Unless otherwise stated, the set of collection markers employed in the completeness and contamination assay is those set forth in Campbell et al. (2013) PNAS USA 110 (14): 5540-45, herein incorporated by reference.

Exemplary steps to obtain a distance estimate between the genomes in question are as follows: (1) Genomes of sufficient quality for comparison must be produced. A genome of sufficient quality is defined as a genome assembly created with enough DNA sequence to amount to at least 20× genome coverage using Illumina HiSeq 2500 technology. The genome must be at least 99% complete with contamination of less than 2% to be compared to the claimed microbe's genome. (2) Genomes are to be compared using the Minhash workflow as demonstrated in Ondov et al. (2016) Genome Biology 17:132, herein incorporated by reference in its entirety. Unless otherwise stated, parameters employed are as follows: “sketch” size of 1000, and “k-mer length” of 21. (3) Confirm that the Mash distance between the two genomes is less than 0.001, 0.0025, 0.005, 0.010, 0.015, 0.020, 0.025, or 0.030. Using the parameters and methods stated above, a Mash distance of 0.015 between two genomes means the expected mutation rate is 0.015 mutations per homologous position. Active variants of the bacteria identified by such methods will retain the ability to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus).

### C. Formulation of a Bacterial Strain

Bacterial strain compositions comprising one or more Bacillus strain of the present disclosure are provided. Such Bacillus strain compositions can (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus). The Bacillus strain composition provided herein can comprise a bacterial cell, a spore, a forespore, and/or a combination of cells, spores, and/or forespores of Bacillus subtilis 750, Bacillus subtilis 747, Bacillus subtilis 839, Bacillus subtilis 1781, Bacillus subtilis 1999, Bacillus subtilis 2018, and/or an active variant thereof. In some embodiments, the Bacillus strain composition comprises Bacillus subtilis 750 or an active variant thereof. The Bacillus strain composition can further comprise a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In specific embodiments, the Bacillus strain composition comprises a bacterial cell, a spore, a forespore, and/or a combination of cells, spores, and/or forespores of Bacillus strain Bacillus subtilis 750 or an active variant thereof and Bacillus strain Bacillus subtilis 747 or an active variant thereof. An effective amount of the Bacillus strain composition can increase conversion of one or more polysaccharides into short chain polysaccharides and/or monosaccharides, increase conversion of one or more proteins into short chain polypeptides and/or amino acids, and/or increase inhibition of growth of one or more pathogens, relative to the absence of said effective amount of said Bacillus strain composition. “One or more polysaccharides” can comprise starch, or one or more non-starch polysaccharides such as arabinoxylan or cellulose. “One or more proteins” can comprise zein or soy protein. In some embodiments, said Bacillus strain composition comprises an activity of arabinoxylanase, cellulase, amylase, zein protease, and/or soy protease. For example, the Bacillus strain composition can increase conversion of arabinoxylan, cellulose, or starch into short chain polysaccharides or monosaccharides, and/or increase conversion of zein or soy protein into short chain polypeptides or amino acids relative to the absence of the Bacillus strain composition.

In some embodiments, the Bacillus strain composition provided herein comprises a bacterial cell, a spore, a forespore, and/or a combination of cells, spores, and/or forespores of (i) Bacillus subtilis 750 or an active variant thereof and (ii) at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. The Bacillus strain composition provided herein can comprise a population (e.g., cells, spores, forespores, or a combination of any thereof) of Bacillus subtilis 750 or an active variant thereof (“Bacillus subtilis 750 population”) and a population (e.g., cells, spores, forespores, or a combination of any thereof) of at least one of Bacillus subtilis 747, Bacillus subtilis 839, Bacillus subtilis 1781, Bacillus subtilis 1999, Bacillus subtilis 2018, or an active variant thereof (“second Bacillus subtilis population”) at any proportions (e.g., ratios). For example, the Bacillus strain composition can comprise the Bacillus subtilis 750 population and the second Bacillus population at the ratio of 1% to 99%, 2% to 98%, 5% to 95%, 10% to 90%, 15% to 85%, 20% to 80%, 25% to 75%, 30% to 70%, 35% to 65%, 40% to 60%, 45% to 55%, 50% to 50%, 55% to 45%, 60% to 40%, 65% to 35%, 70% to 30%, 75% to 25%, 80% to 20%, 85% to 15%, 90% to 10%, 95% to 5%, 98% to 2%, or 99% to 1%. In specific embodiments, the Bacillus strain composition comprises the Bacillus subtilis 750 population the second Bacillus population in equal proportions (e.g., 50% to 50%, or within 0, 1, 2, 3, 4, 5, 6, 7, 8, 9, 10, 11, 12, 13, 14, 15, 16, 17, 18, 19, or 20% difference between each other, such that the Bacillus subtilis 750 population and the second Bacillus population are in substantially equal proportions).

In some embodiments, the Bacillus strain composition provided herein comprises a bacterial cell, a spore, a forespore, and/or a combination of cells, spores, and/or forespores of Bacillus subtilis 750 or an active variant thereof and Bacillus subtilis 747 or an active variant thereof. The Bacillus strain composition provided herein can comprise a population (e.g., cells, spores, forespores, or a combination of any thereof) of Bacillus subtilis 747 or an active variant thereof (“Bacillus subtilis 747 population”) and a population (e.g., cells, spores, forespores, or a combination of any thereof) of Bacillus subtilis 750 (“Bacillus subtilis 750 population”) at any proportions (e.g., ratios). For example, the Bacillus strain composition can comprise the Bacillus subtilis 747 population and the Bacillus subtilis 750 population at the ratio of 1% to 99%, 2% to 98%, 5% to 95%, 10% to 90%, 15% to 85%, 20% to 80%, 25% to 75%, 30% to 70%, 35% to 65%, 40% to 60%, 45% to 55%, 50% to 50%, 55% to 45%, 60% to 40%, 65% to 35%, 70% to 30%, 75% to 25%, 80% to 20%, 85% to 15%, 90% to 10%, 95% to 5%, 98% to 2%, or 99% to 1%. In specific embodiments, the Bacillus strain composition comprises the Bacillus subtilis 747 population the Bacillus subtilis 750 population in equal proportions (e.g., 50% to 50%, or within 0, 1, 2, 3, 4, 5, 6, 7, 8, 9, 10, 11, 12, 13, 14, 15, 16, 17, 18, 19, or 20% difference between each other, such that the Bacillus subtilis 750 population and the second Bacillus population are in substantially equal proportions).

The Bacillus strain composition provided herein can comprise Bacillus strains (e.g., Bacillus subtilis 750, Bacillus subtilis 747, Bacillus subtilis 839, Bacillus subtilis 1781, Bacillus subtilis 1999, Bacillus subtilis 2018, and/or an active variant thereof), at least one of which can be a powdered and/or lyophilized strain. Lyophilization, or freeze-drying, refers to a process of removing the water from a sample, by freezing and then drying the sample under a vacuum at low temperatures. In the process of lyophilization, the water component in bacterial cells is removed, transforming them into a non-naturally occurring product. Lyophilized Bacillus strains or compositions comprising one or more lyophilized Bacillus strains do not exist in nature and can have significantly increased stability, shelf life, and/or storage duration relative to a non-lyophilized or naturally occurring Bacillus strain counterpart, or a composition comprising the same. For example, a lyophilized Bacillus strain or a composition comprising the same can have stability, shelf life, and/or storage duration that is increased, e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a non-lyophilized or naturally occurring Bacillus strain or composition counterpart. When germinated, a lyophilized Bacillus strain or a composition comprising the same can have significantly different (e.g., increased) biological function, e.g., ability to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus), relative to a non-lyophilized or naturally occurring Bacillus strain or composition counterpart stored for the same duration of time. For example, when germinated, a lyophilized Bacillus strain or a composition comprising the same can have an enzymatic activity to convert one or more polysaccharides or proteins and/or a pathogen inhibitory activity that is increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a non-lyophilized or naturally occurring Bacillus strain or composition counterpart stored for the same duration of time. The enzymatic activity (e.g., arabinoxylanase, cellulase, amylase, zein protease, or soy protease activity) can be measured by standard methods, such as functional assays to measure conversion of the substrate (e.g., polysaccharide, protein) to product (e.g., short chain polysaccharides, monosaccharides, short chain polypeptides, amino acids) by gas chromatography, mass spectrometry, colorimetric assays, or tracer assays. The pathogen inhibitory activity can be measured by standard methods, such as measuring CFUs or number of bacteria in a sample with and without administration of the Bacillus strain composition.

The Bacillus strain composition provided herein can further comprise a cryoprotectant. A “cryoprotectant” as used herein refers to a compound that prevents damage to cells (e.g., bacterial cells) or bacterial strain during freezing. Exemplary cryoprotectants include skim milk (e.g., 10%), glycerin (e.g., 15%), a natural deep eutectic solvent, ice recrystallization inhibitors (e.g., vinyl alcohol, ethylene glycol), sucrose, trehalose, and sodium glutamate. In specific embodiments, the cryoprotectant is not naturally-occurring (i.e., not found in nature) and/or does not naturally occur with the Bacillus strain such that the bacterial strain composition comprising the cryoprotectant is not naturally occurring. In specific embodiments, the cryoprotectant that does not naturally occur with the Bacillus strain improves at least one property of the strain such as stability, activity over time, or solubilization. The Bacillus strain composition provided herein, comprising a cryoprotectant, can be stored at a freezing temperature (e.g., −80° C., −20° C.) (i.e., cryopreservation). Cryopreservation of the Bacillus strain or the Bacillus strain composition provided herein allows for a long storage time, wide range of applications, low biological mutation rate, and/or increased activity to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus) as compared to a control composition not comprising a cryoprotectant. In some embodiments, the ability of the Bacillus strain composition comprising a cryoprotectant to (i) convert one or more polysaccharides into short chain polysaccharides and/or monosaccharides and/or convert one or more proteins into short chain polypeptides and/or amino acids, and/or (ii) inhibit growth of one or more pathogens is increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a control Bacillus strain composition comprising no cryoprotectant. The enzymatic activity (e.g., arabinoxylanase, cellulase, amylase, zein protease, or soy protease activity) can be measured by standard methods, such as functional assays to measure conversion of the substrate (e.g., polysaccharide, protein) to product (e.g., short chain polysaccharides, monosaccharides, short chain polypeptides, amino acids) by gas chromatography, mass spectrometry, colorimetric assays, or tracer assays. The pathogen inhibitory activity can be measured by standard methods, such as measuring CFUs or number of bacteria in a sample with and without administration of the Bacillus strain composition.

The Bacillus strain composition provided herein can further comprise a preservative. A “preservative” as used herein refers to a compound that facilitates preservation or prevents decay of cells (e.g., bacterial cells) or bacterial strain. Exemplary preservatives include sulfites (e.g., sodium sulfite, sodium bisulfite, sodium metabisulfite, potassium bisulfite, potassium metabisulfite, sulfur dioxide), benzoates (e.g., sodium benzoate), and nitrites (e.g., sodium nitrite). In specific embodiments, the preservative is not naturally-occurring (i.e., not found in nature) and/or does not naturally occur with the Bacillus strain such that the bacterial strain composition comprising the preservative is not naturally occurring. In specific embodiments, the preservative that does not naturally occur with the Bacillus strain improves at least one property of the strain such as stability, activity over time, or solubilization. The Bacillus strain composition comprising a preservative can have increased activity to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus) as compared to a control composition not comprising a preservative. In some embodiments, the activity of the Bacillus strain composition to (i) convert one or more polysaccharides into short chain polysaccharides and/or monosaccharides and/or convert one or more proteins into short chain polypeptides and/or amino acids, and/or (ii) inhibit growth of one or more pathogens comprising a preservative is increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a control Bacillus strain composition comprising no preservative. The enzymatic activity (e.g., arabinoxylanase, cellulase, amylase, zein protease, or soy protease activity) can be measured by standard methods, such as functional assays to measure conversion of the substrate (e.g., polysaccharide, protein) to product (e.g., short chain polysaccharides, monosaccharides, short chain polypeptides, amino acids) by gas chromatography, mass spectrometry, colorimetric assays, or tracer assays. The pathogen inhibitory activity can be measured by standard methods, such as measuring CFUs or number of bacteria in a sample with and without administration of the Bacillus strain composition.

The Bacillus strain composition provided herein can further comprise a carrier. As used herein, the term “carrier” refers to an inert compound that is compatible with any other ingredients in the formulation and is not deleterious to the active compound (i.e., bacterial strains) or a subject that the formulation is administered thereto. In specific embodiments, the carrier is not naturally-occurring (i.e., not found in nature) and/or does not naturally occur with the Bacillus strain such that the resulting bacterial strain composition is not naturally occurring. In specific embodiments, a carrier that does not naturally occur with the Bacillus strain improves at least one property of the strain such as recovery, efficacy, physical properties, packaging, or administration. Non-limiting examples of carriers include inert diluents (e.g., sodium and calcium carbonate, sodium and calcium phosphate, and lactose), disintegrating agents (e.g., corn starch, alginic acid), binding agents (e.g., starch, gelatin, cellulose, whey powder, rice hulls), lubricating agents (e.g., magnesium stearate, stearic acid, talc), sweetening agents, flavoring agents, coloring agents, coating agents (e.g., glyceryl monostearate, glyceryl distearate).

In some embodiments, the bacterial strain composition comprises a pharmaceutical composition wherein the bacterial strains provided herein are formulated as a pharmaceutical composition along with a pharmaceutically acceptable carrier. Such pharmaceutically acceptable carriers are known in the art and include an inert vehicle, adjuvants, preservatives etc. In some embodiments, the pharmaceutically acceptable carrier comprises one that is not naturally-occurring (i.e., not found in nature) and/or does not naturally occur with the Bacillus strain (i.e., not found with a cell, spore, or forespore of the Bacillus strain in the native environment of the Bacillus strain) such that the resulting bacterial strain composition is not naturally occurring. Such pharmaceutical compositions can be prepared in accordance with techniques such as those provided in Remington, The Science and Practice of Pharmacy (21st ed. 2005).

The carrier(s) or pharmaceutically acceptable carrier(s) may comprise about 30% weight per weight, weight per volume, or volume per volume, of the final composition. In some embodiments, the carrier(s) or pharmaceutically acceptable carrier(s) may comprise about 40%, about 50%, about 60%, about 70%, about 80%, about 90%, about 91%, about 92%, about 93%, about 94%, about 95%, about 96%, about 97%, about 98%, about 98.5%, about 99.0%, about 99.5%, or about 99.9% weight per weight, weight per volume, or volume per volume of the final composition.

In some embodiments, the Bacillus strain composition further comprises one or more polysaccharides (e.g., arabinoxylan, cellulose, starch) and/or one or more proteins (e.g., zein, soy protein). The Bacillus strain composition can comprise one or more polysaccharides and/or one or more proteins, and a Bacillus strain comprising a bacterial cell, a spore, a forespore, and/or a combination of cells, spores, and/or forespores of Bacillus subtilis 747 or an active variant thereof, and a bacterial cell, a spore, a forespore, and/or a combination of cells, spores, and/or forespores of Bacillus subtilis 750 or an active variant thereof. In some embodiments, the Bacillus strain composition further comprises feed, forage, or fodder comprising said one or more polysaccharides or said one or more proteins. In some embodiments, Bacillus strain composition comprises one or more polysaccharides; one or more amino acids; or feed, forage, or fodder comprising one or more polysaccharides or one or more proteins, and further comprises one or more enzymes that catalyzes conversion of the one or more polysaccharides into short chain polysaccharides and/or monosaccharides (e.g., xylanase, arabinoxylanase, amylase, cellulose) or one or more enzymes that catalyzes conversion of the one or more proteins into one or more short chain polypeptides and/or amino acids (e.g., zein protease, soy protease). The one or more enzymes (e.g., xylanase, arabinoxylanase, amylase, cellulose, zein protease, soy protease) can be exogenously added to the one or more polysaccharides, one or more amino acids, feed, forage, fodder, or the Bacillus strain composition to catalyze conversion of the one or more polysaccharides (e.g., xylan, arabinoxylan, starch, cellulose) or one or more proteins (e.g., zein, soy protein). As used herein, the “feed” or “feedstuff” may comprise feed materials comprising maize or corn, wheat, barley, triticale, rye, rice, tapioca, sorghum, and/or any of the by-products, as well as protein rich components like soybean meal, rape seed meal, canola meal, cotton seed meal, sunflower seed meal, cornmeal, animal-by-product meals, and mixtures thereof. The feed may comprise animal fats and/or vegetable oils. The feed may also contain additional minerals such as, for example, calcium and/or additional vitamins. barley, wheat, silage, and haylage. “Forage” as used herein refers to any crops that animals graze independently. Forage crops can include grasses (e.g., Agrostis spp., Agrostis capillaris, Agrostis stolonifera, Andropogon hallii, Arrhenatherum elatius, Bothriochloa bladhii, Bothriochloa pertusa, Brachiaria decumbens, Brachiaria humidicola, Bromus spp., Cenchrus ciliaris, Chloris gayana, Cynodon dactylon, Dactylis glomerata, Echinochloa pyramidalis, Entolasia imbricata, Festuca spp., Festuca arundinacea, Festuca pratensis, Festuca rubra, Heteropogon contortus, Hymenachne amplexicaulis, Hyparrhenia rufa, Leersia hexandra, Lolium spp., Lolium multiflorum, Lolium perenne, Megathyrsus maximus, Melinis minutiflora, Paspalum conjugatum, Paspalum dilatatum, Phalaris arundinacea, Phleum pratense, Poa spp., Poa arachnifera, Poa pratensis, Poa trivialis, Setaria sphacelata, Themeda triandra, Thinopyrum intermedium) and legumes (Arachis pintoi, Astragalus cicer, Chamaecrista rotundifolia, Clitoria ternatea, Kummerowia, Lotus corniculatus, Macroptilium atropurpureum, Macroptilium bracteatum, Medicago spp., Melilotus spp., Neonotonia wightii, Onobrychis viciifolia, Stylosanthes spp., Trifolium spp., Vicia spp., Acacia aneura, Albizia spp., Enterolobium cyclocarpum, Leucaena leucocephala), and can have purposes other than animal feed. As used herein, “fodder” refers to any food that is provided to an animal rather than the animal having to forage for it themselves. Fodder encompasses plants that have been cut. Fodder can include hay, straw, silage (e.g., alfalfa, maize, grass-legume mix, sorghums, oats), crop residue, compressed and pelleted feeds, oils, mixed rations, sprouted grains, and legumes.

In some embodiments, an effective amount of the Bacillus strain composition, comprising one or more polysaccharides and/or proteins, or feed, forage, or fodder comprising the polysaccharides and/or proteins increases conversion of (i) one or more polysaccharides into short chain polysaccharides and/or monosaccharides and/or (ii) one or more proteins into short chain polypeptides and/or amino acids relative to the absence of said effective amount of said Bacillus strain composition.

Also provided herein is a reduced energy diet. As used herein, a “reduced energy diet” refers to a diet for an animal (e.g., birds, swine, ruminants) that comprises lower apparent metabolizable energy (AME) relative to a control diet or a normal energy diet comprising AME required to maintain performance and/or body weight of the animal. The reduced energy diet can have reduction in the content of fat, carbohydrate, protein, or combination of any thereof, or substitution of one or more of fat, carbohydrate, and protein with one or more other energy sources (e.g., substitution of fat with carbohydrate, e.g., substitution of soybean oil with corn). The reduced energy diet can have AME that is reduced by about 0.5-5%, 5-10%, 10-20%, 20-30%, or more, e.g., about 0.5%, 1%, 1.5%, 2%, 3%, 4%, 5%, 6%, 7%, 8%, 9%, 10%, 15%, 20%, 30%, or more, e.g., at least about 0.5%, 1%, 1.5%, 2%, 3%, 4%, 5%, 6%, 7%, 8%, 9%, 10%, 15%, 20%, 30% as compared to a control diet or a normal energy diet. In specific embodiments, a prestarter reduced energy diet comprises 50 kcal or 75 kcal less AME (e.g., per kg of feed) as compared to a control prestarter diet having 2,900 kcal AME or 3,000 kcal AME (e.g., per kg of feed). In further embodiments, a starter reduced energy diet comprises 50 kcal or 75 kcal less AME (e.g., per kg of feed) as compared to a control starter diet having 3,000 kcal AME or 3,100 kcal AME (e.g., per kg of feed). In further embodiments, a finisher reduced energy diet comprises 50 kcal or 75 kcal less AME (e.g., per kg of feed) as compared to a control starter diet having 3,050 kcal AME or 3,200 kcal AME (e.g., per kg of feed). In some embodiments, a reduced energy diet comprises the Bacillus strain composition provided herein (e.g., comprising Bacillus subtilis 750 or an active variant thereof; comprising at least one of Bacillus subtilis 750, Bacillus subtilis 747, Bacillus subtilis 839, Bacillus subtilis 1781, Bacillus subtilis 1999, Bacillus subtilis 2018, or an active variant thereof, or a combination of any thereof; comprising a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; or comprising Bacillus subtilis 750 or an active variant thereof and Bacillus subtilis 747 or an active variant thereof). Animals fed a reduced energy diet comprising the Bacillus strain composition can maintain performance and/or body weight that is comparable to animals fed a control (e.g., normal or non-reduced energy) diet, and can have increased nutrition efficiency, increased nutrient absorption, increased or comparable integrity of a gastrointestinal barrier, increased or comparable mucosal immunity (e.g., level of secretory IgA), reduced or comparable absence of inflammation, reduced or comparable levels or acute phase proteins (e.g., α-1-AGP) and/or inflammatory cytokines (e.g., IL-6), and/or reduced amount or diversity of pathogens in the gastrointestinal tract relative to animals fed a control (e.g., normal) diet not comprising the Bacillus strain composition. Animals fed a reduced energy diet not comprising the Bacillus strain composition can have decreased nutrient absorption, increased hindgut fermentation, decreased integrity of a gastrointestinal barrier, increased gastrointestinal permeability, decreased mucosal immunity (e.g., level of secretory IgA), increased inflammation, increased levels or acute phase proteins (e.g., a-1-AGP) and/or inflammatory cytokines (e.g., IL-6), and/or an increased amount or diversity of pathogens in the gastrointestinal tract of the animal relative to animals fed a normal diet without the Bacillus strain composition. Animals fed a reduced energy diet comprising the Bacillus strain composition can have increased nutrition efficiency, increased nutrient absorption, reduced availability of nutrients in the hindgut of the animal for fermentation, increased integrity of a gastrointestinal barrier in the animal, increases mucosal immunity (e.g., level of secretory IgA), reduced inflammation, reduced levels or acute phase proteins (e.g., α-1-AGP) and/or inflammatory cytokines (e.g., IL-6), and/or reduced amount or diversity of pathogens in the gastrointestinal tract relative to animals fed the reduced energy diet not comprising the Bacillus strain composition. In specific embodiments, chickens fed ad libitum a reduced energy diet comprising 50 kcal less apparent metabolizable energy per kg of feed supply relative to a normal diet, and supplemented with the Bacillus composition provided herein, can maintain performance and/or body weight comparable to control chickens fed a normal diet.

In some embodiments, feed, forage, fodder, or food compositions can be prepared by combining a formulated bacterial strain of the invention with typical animal feed, forage, fodder, food, or drink ingredients. A formulated bacterial strain of the invention can be used for the preparation of animal feed, forage, fodder, or food products or beverages, and/or may be added to drinking or rearing water. In other embodiments, the compositions of the present invention are additives that are added to an animal's feed, forage, fodder, food, drinking water, or beverage prior to ingestion.

The Bacillus strain composition provided herein can be formulated as feed (e.g., as pellets, mash, crumble, cake, meal); as feed, forage, fodder, food, or drink additives (e.g., as powder, liquid, capsule, gel, paste, cream, tablet); in combinations with food or beverage items; in combination with one or more polysaccharides or proteins; in oils; as a suppository; in lubricants; as sachets; or for other administration routes. In some embodiments, the bacterial strain composition disclosed herein is formulated as a liquid formulation or a solid formulation. When the bacterial strain composition is a solid formulation, it may be formulated as a tablet, a sucking tablet, a chewing tablet, a chewing gum, a capsule, a sachet, a powder, a granule, a pellet, a cell pellet, a dust, a coated particle, a coated tablet, an enterocoated tablet, an enterocoated capsule, a melting strip, or a film. When the bacterial strain composition is a liquid formulation, it may be formulated as an aqueous solution, an oil-based liquid product, a gel, a suspension, an emulsion, a slurry, a paste, a cell paste, a cream, an ointment, or syrup. The composition may further comprise a carrier material independently selected from, but not limited to, the group consisting of vegetables, lactic acid fermented foods, fermented dairy products, resistant starch, dietary fibers, carbohydrates, proteins, and glycosylated proteins.

The various compositions and formulations disclosed herein can comprise an amount of the Bacillus strains (i.e., cells of Bacillus subtilis 750, Bacillus subtilis 747, Bacillus subtilis 839, Bacillus subtilis 1781, Bacillus subtilis 1999, Bacillus subtilis 2018, or an active variant thereof, or spores or forespores or a combination of cells, forespores and/or spores, formed from Bacillus subtilis 750, Bacillus subtilis 747, Bacillus subtilis 839, Bacillus subtilis 1781, Bacillus subtilis 1999, Bacillus subtilis 2018, or an active variant thereof, e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, Bacillus subtilis 2018, or an active variant thereof, or a combination of any thereof; a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; or Bacillus subtilis 747 or an active variant thereof and Bacillus subtilis 750 or an active variant thereof). The concentrations of the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof; a combination of Bacillus subtilis 747 or an active variant thereof and Bacillus subtilis 750 or an active variant thereof) can comprise about 10⁴ CFU/g to about 10¹⁰ CFU/g, about 10⁴ CFU/g to about 10¹¹ CFU/g, at least about 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 10⁵ CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, about 10⁹ CFU/g to about 10¹⁰ CFU/g, about 10⁵ CFU/g to about 10⁹ CFU/g, about 10⁵ CFU/g to about 10⁸ CFU/g, about 10⁵ CFU/g to about 10⁷ CFU/g, or about 10⁵ CFU/g to about 10⁶ CFU/g. In other embodiments, the concentrations of the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof, or a combination of any thereof; a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; a combination of Bacillus subtilis 747 or an active variant thereof and Bacillus subtilis 750 or an active variant thereof) comprises or consists of at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g. In liquid compositions and formulations, the bacterial strain(s) of the composition can comprise a concentration (e.g., of the two bacterial strains together) of about 10⁴ CFU/ml to about 10¹² CFU/ml, 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, about 10⁹ CFU/ml to about 10¹⁰ CFU/ml, about 10⁵ CFU/ml to about 10⁹ CFU/ml, about 10⁵ CFU/ml to about 10⁸ CFU/ml, about 10⁵ CFU/ml to about 10⁷ CFU/ml, or about 10⁵ CFU/ml to about 10⁶ CFU/ml. In other embodiments, the concentration of at least one of the bacterial strains provided herein or active variant thereof comprises or consists of at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml. Without wishing to be bound by theory, the total Bacillus concentrations in the natural environment, e.g., in regular feed prepared by standard methods such as by mixing bulk ingredients (e.g., corn and soybean) and micro ingredients (e.g., amino acids and minerals, but not a bacterial strain composition), can range from about 1×10³ CFU/g to about 2×10⁵ CFU/g or about 1×10³ CFU/ml to about 2×10⁵ CFU/ml, but no more than about 5×10⁵ CFU/g or about 5×10⁵ CFU/ml. Further, there is no evidence that any of Bacillus subtilis 750, Bacillus subtilis 747, Bacillus subtilis 839, Bacillus subtilis 1781, Bacillus subtilis 1999, or Bacillus subtilis 2018 exist outside of a man-made environment, where there are less or no exposure to the selective pressure of stresses and pathogens resulting from the conditions of the artificial environment. Bacillus subtilis 750, Bacillus subtilis 747, Bacillus subtilis 839, Bacillus subtilis 1781, Bacillus subtilis 1999, and Bacillus subtilis 2018 were isolated under the selective pressure of a commercial farm in which the animals are kept in a man-made environment, largely fed a man-made, rationed diet, exposed to the pathogens and stresses resulting from the conditions of the non-natural environment, and administered non-natural interventions such as medications. In specific embodiments, the effective amount of the Bacillus strain composition comprises the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, an active variant thereof, or a combination of any thereof; a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; a combination of Bacillus subtilis 750 or active variant thereof and Bacillus subtilis 747 or active variant thereof) at a concentration that does not occur in nature (e.g., at a higher concentration than that found in nature), and such non-naturally occurring effective amount of the Bacillus strains confers the composition the increased activity to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus) as compared to feed, forage, or fodder not containing the non-naturally occurring effective amount of the Bacillus strains. In specific embodiments, the effective amount of the Bacillus strain composition comprises the bacterial strain(s) in a non-naturally occurring concentration of about 1.5×10⁵ CFU/gram of composition to about 1.5×10⁶ CFU/gram of composition. In further embodiments, the effective amount of the Bacillus strain composition comprises Bacillus subtilis 750 or an active variant thereof in a non-naturally occurring concentration of about 1.5×10⁵ CFU/gram of composition to about 1.5×10⁶ CFU/gram of composition. In further embodiments, the effective amount of the Bacillus strain composition comprises (i) Bacillus subtilis 750 or an active variant thereof and (ii) at least one of Bacillus subtilis 747, Bacillus subtilis 839, Bacillus subtilis 1781, Bacillus subtilis 1999, or Bacillus subtilis 2018, or an active variant thereof, with optionally (i) and (ii) in equal proportions, in a total concentration of about 1.5×10⁵ CFU/gram of composition to about 1.5×10⁶ CFU/gram of composition, which is a non-naturally occurring concentration. In further specific embodiments, the effective amount of the Bacillus strain composition comprises Bacillus subtilis 747 and Bacillus subtilis 750, optionally in equal proportions, in a total concentration of about 1.5×10⁵ CFU/gram of composition to about 1.5×10⁶ CFU/gram of composition, which is a non-naturally occurring concentration.

Feed, forage, or fodder comprising the Bacillus strain composition provided herein can be prepared by mixing the Bacillus strain composition comprising the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof; a combination of Bacillus subtilis 747 or active variant thereof and/or Bacillus subtilis 750 and active variant thereof) that has non-naturally occurring concentrations (e.g., about 1×10⁷ CFU/g-1×10⁸ CFU/g, 1×10⁸ CFU/g-1×10⁹ CFU/g, 1×10⁹ CFU/g-1×10¹⁰ CFU/g, 1×10¹⁰ CFU/g-1×10¹¹ CFU/g, 1×10¹¹ CFU/g-1×10¹² CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, at least about 10¹² CFU/g, about 1×10⁷ CFU/ml-1×10⁸ CFU/ml, 1×10⁸ CFU/ml-1×10⁹ CFU/ml, 1×10⁹ CFU/ml-1×10¹⁰ CFU/ml, 1×10¹⁰ CFU/ml-1×10¹¹ CFU/ml, 1×10¹¹ CFU/ml-1×10¹² CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml) of the Bacillus strain(s) with the ingredients of the regular feed, forage, or fodder. Accordingly, the resulting feed, forage, or fodder comprises the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof, or a combination of any thereof; a combination of Bacillus subtilis 747 or an active variant thereof and Bacillus subtilis 750 or an active variant thereof) at non-naturally occurring concentrations. For example, the feed, forage, or fodder (collectively “food”) can comprise the Bacillus strain(s) at non-naturally occurring concentrations of at least about 10⁴ CFU/gram of food to about 10¹⁰ CFU/gram of food, 10⁴ CFU/gram of food to about 10¹¹ CFU/gram of food, 10⁴ CFU/gram of food to about 10¹² CFU/gram of food, about 10⁵ CFU/gram of food to about 10¹² CFU/gram of food, about 10⁶ CFU/gram of food to about 10¹² CFU/gram of food, about 10⁷ CFU/gram of food to about 10¹² CFU/gram of food, about 10⁸ CFU/gram of food to about 10¹² CFU/gram of food, about 10⁹ CFU/gram of food to about 10¹² CFU/gram of food, about 10¹⁰ CFU/gram of food to about 10¹² CFU/gram of food, about 10¹¹ CFU/gram of food to about 10¹² CFU/gram of food, about 10⁵ CFU/gram of food to about 10¹¹ CFU/gram of food, about 10⁵ CFU/gram of food to about 10¹¹ CFU/gram of food, about 10⁶ CFU/gram of food to about 10¹¹ CFU/gram of food, about 10⁷ CFU/gram of food to about 10¹¹ CFU/gram of food, about 10⁸ CFU/gram of food to about 10¹¹ CFU/gram of food, about 10⁹ CFU/gram of food to about 10¹¹ CFU/gram of food, about 10¹⁰ CFU/gram of food to about 10¹¹ CFU/gram of food, about 10⁵ CFU/gram of food to about 10¹⁰ CFU/gram of food, about 10⁶ CFU/gram of food to about 10¹⁰ CFU/gram of food, about 10⁷ CFU/gram of food to about 10¹⁰ CFU/gram of food, about 10⁸ CFU/gram of food to about 10¹⁰ CFU/gram of food, about 10⁹ CFU/gram of food to about 10¹⁰ CFU/gram of food, about 10⁵ CFU/gram of food to about 10⁹ CFU/gram of food, about 10⁵ CFU/gram of food to about 10⁸ CFU/gram of food, about 10⁵ CFU/gram of food to about 10⁷ CFU/gram of food, or about 10⁵ CFU/gram of food to about 10⁶ CFU/gram of food, e.g., at least about 10⁴ CFU/gram of food, at least about 10⁵ CFU/gram of food, at least about 10⁶ CFU/gram of food, at least about 10⁷ CFU/gram of food, at least about 10⁸ CFU/gram of food, at least about 10⁹ CFU/gram of food, at least about 10¹⁰ CFU/gram of food, at least about 10¹¹ CFU/gram of food, or at least about 10¹² CFU/gram of food. In specific embodiments, the effective amount of feed, forage, or fodder provided herein comprises the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof, or combination of any thereof; a combination of Bacillus subtilis 747 or an active variant thereof and Bacillus subtilis 750 or an active variant thereof) at a concentration that does not occur in nature (e.g., at a higher concentration than that found in nature), and such non-naturally occurring effective amount of the Bacillus strains enables (i) increased conversion of one or more polysaccharides into short chain polysaccharides and/or monosaccharides and/or convert one or more proteins into short chain polypeptides and/or amino acids, and/or (ii) increased inhibition of growth of one or more pathogens (e.g., Escherichia coli, Clostridium, Salmonella, and Streptococcus) as compared to feed, forage, or fodder not containing the non-naturally occurring effective amount of the Bacillus strains. In specific embodiments, the effective amount of the Bacillus strain composition comprises the bacterial strain(s)) in a non-naturally occurring concentration of about 1.5×10⁵ CFU/gram of food to about 1.5×10⁶ CFU/gram of food. In specific embodiments, the effective amount of the Bacillus strain composition comprises Bacillus subtilis 750 or an active variant thereof in a total concentration of about 1.5×10⁵ CFU/gram of food to about 1.5×10⁶ CFU/gram of food, which is a non-naturally occurring concentration. In other embodiments, the effective amount of the Bacillus strain composition comprises (i) Bacillus subtilis 750 or an active variant thereof and (ii) at least one of Bacillus subtilis 747, Bacillus subtilis 839, Bacillus subtilis 1781, Bacillus subtilis 1999, or Bacillus subtilis 2018, or an active variant thereof, optionally with (i) and (ii) in equal proportions, in a total concentration of about 1.5×10⁵ CFU/gram of food to about 1.5×10⁶ CFU/gram of food, which is a non-naturally occurring concentration. In further specific embodiments, the effective amount of the Bacillus strain composition comprises Bacillus subtilis 747 and Bacillus subtilis 750, optionally in equal proportions, in a total concentration of about 1.5×10⁵ CFU/gram of food to about 1.5×10⁶ CFU/gram of food, which is a non-naturally occurring concentration.

Additional beneficial microbes may be combined with a bacterial strain of the invention into a formulated product. Alternatively, additional formulated microbes may be combined or mixed with a formulated bacterial strain of the invention into a feed, fodder, or food composition, into drinking water, or into a pharmaceutical composition. Alternatively, the additional microbes may be administered at a different time. The additional beneficial microbes can exhibit an additional or synergistic effect with the Bacillus strain and/or Bacillus strain composition provided herein to (i) increase production of short chain polysaccharides, monosaccharides, short chain polypeptides, and/or amino acids and/or (ii) increase inhibition of pathogens (e.g., Escherichia coli, Clostridium, Salmonella, and Streptococcus). The additional beneficial microbes can exhibit an additional health promoting effect to animals. These additional beneficial microbes may be selected from species of Saccharomyces, species of Bacillus such as Bacillus subtilis, Bacillus licheniformis, Bacillus lentus, Bacillus pumilus, Bacillus laterosporus, Bacillus coagulans, Bacillus alevi, Bacillus cereus, Bacillus clausii, Bacillus coagulans, Bacillus inaquosorum, Bacillus mojavensis, Bacillus velezensis, Bacillus vallismortis, Bacillus amyloliquefaciens, Bacillus atropheus, Bacillus altitudinis, Bacillus safensis, Bacillus alcalophilus, Bacillus badius, or Bacillus thurigiensis; from species of Enterococcus such as Enterococcus faecium; from species of Clostridium such as Clostridium butyricum; from species of Lactococcus such as Lactococcus lactis or Lactoccus cremoris; from species of Bifidobacterium such as Bifidobacterium adolescentis, Bifidobacterium animalis, Bifidobacterium bifidum, Bifidobacterium infantis, Bifidobacterium longum, Bifidobacterium pseudolongum, or Bifidobacterium thermophilum; from species of Lactobacillus such as Lactobacillus alactosus, Lactobacillus alimentarius, Lactobacillus amylovorans, Lactobacillus amylophilus, Lactobacillus amylovorans, Lactobacillus acidophilus, Lactobacillus agilis, Lactobacillus animalis, Lactobacillus batatas, Lactobacillus bavaricus, Lactobacillus bifermentans, Lactobacillus bidifus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus curvatus, Lactobacillus coprohilus, Lactobacillus delbrueckii, Lactobacillus fermentum, Lactobacillus gasseri, Lactobacillus jugurti, Lactobacillus kefir, Lactobacillus lactis, Lactobacillus leichmannii, Lactobacillus mali, Lactobacillus malefermentans, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus tolerans, Lactobacillus torquens, Lactobacillus ruminis, Lactobacillus sake, Lactobacillus saliverius, Lactobacillus sharpeae, Lactobacillus sobrius, Lactobacillus trichodes, Lactobacillus vaccinostercus, Lactobacillus viridescens, Lactobacillus vitulinus, Lactobacillus xylosus, Lactobacillus yamanashiensis, or Lactobacillus zeae; from species of Megasphaera such as Megasphaera elsdenil; from species of Prevotella such as Prevotella bryantii; from species of Pediococcus such as Pediococcus acidilactici, or Pediococcus pentosaceus; from species of Streptococcus such as Streptococcus cremoris, Streptococcus discetylactis, Streptococcus faecium, Streptococcus lactis, Streptococcus thermophilus, or Streptococcus intermedius; or from species of Propionibacterium such as Propionibacterium freudenreichii, Propionibacterium acidipropionici, Propionibacterium jensenii, Propionibacterium thoenii, Propionibacterium australiense, or Propionibacterium avidum, and/or a combination thereof.

Compositions of the invention may also include prebiotics, which may be combined or mixed with a formulated bacterial strain of the invention into a feed or food composition, into drinking water, or into a pharmaceutical composition. Prebiotics are food ingredients that are not readily digestible by enzymes endogenous to the gut (such as those expressed by the animal or those expressed by the resident gut microbiome) and that selectively stimulate the growth and activity of selected groups of intestinal microorganisms that confer beneficial effects upon their host. Typically, it is beneficial microorganism populations that benefit from the presence of prebiotic compounds. Prebiotics can consist of oligosaccharides and other small molecules that serve as metabolic substrates for growth of beneficial microbes. Common prebiotics include galacto-oligosaccharides, fructo-oligosaccharides, inulin, isomalto-oligosaccharies, gentio-oligosaccharides, lactilol, lactosucrose, lactulose, xylosucrose, glycosylsucrose, pyrodextrins, soybean oligosaccharides, guar gum, locust bean gum, arabinan, galactan, pectins, and pectic polysaccharides. While many diverse microbes inhabit the intestinal tract of a host organism, prebiotic compounds are only utilized by the beneficial microbes and lead to a selective enhancement of the beneficial microbe population. A formulation that includes both prebiotics and probiotics may be known as a “synbiotic”.

Bacterial compositions of the invention can comprise an enzyme potentiator (i.e., cofactor). Enzyme potentiators may be used to enhance the activity of enzymes to convert one or more polysaccharides into short chain polysaccharides or monosaccharides or to convert one or more proteins into short chain polypeptides or amino acids, e.g., arabionxylanase, cellulase, amylase, zein protease, or soy protease activities, or pathogen inhibitory activity. In some embodiments, an enzyme potentiator such as ascorbic acid (vitamin C) facilitates the conversion reaction to sulforaphane to occur in the location needed for peak absorption. The enzyme potentiator may be obtained from a natural source, or it may be produced synthetically.

### IV. Methods Comprising a Bacterial Strain

Methods are provided herein for increasing production of short chain polysaccharides, monosaccharides, short chain polypeptides, and/or amino acids. The method can comprise contacting one or more polysaccharides (e.g., arabinoxylan, cellulose, starch) and/or one or more proteins (e.g., zein, soy protein) with an effective amount of a Bacillus strain composition provided herein. The Bacillus strain composition can comprise Bacillus subtilis 750, Bacillus subtilis 747, Bacillus subtilis 839, Bacillus subtilis 1781, Bacillus subtilis 1999, Bacillus subtilis 2018, and/or an active variant thereof. In some embodiments, the Bacillus strain composition comprises Bacillus subtilis 750 or an active variant thereof. The Bacillus strain composition can further comprise a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. In specific embodiments, the Bacillus strain composition comprises a bacterial cell, a spore, a forespore, and/or a combination of cells, spores, and/or forespores of Bacillus strain Bacillus subtilis 750 or an active variant thereof and Bacillus strain Bacillus subtilis 747 or an active variant thereof. In specific embodiments, the Bacillus strain composition comprises a bacterial cell, a spore, a forespore, and/or a combination of cells, spores, and/or forespores of Bacillus subtilis 747 or an active variant thereof, and a bacterial cell, a spore, a forespore, and/or a combination of cells, spores, and/or forespores of Bacillus subtilis 750 or an active variant thereof. In some embodiments, the method comprises contacting feed, forage, or fodder comprising one or more polysaccharides and/or one or more proteins with the effective amount of said Bacillus strain composition. The effective amount of the Bacillus strain composition can increase production of short chain polysaccharides, monosaccharides, short chain polypeptides, and/or amino acids, and/or inhibit growth of one or more pathogens (e.g., Escherichia coli, Clostridium, Salmonella, and Streptococcus). For example, contacting one or more polysaccharides or proteins with the effective amount of the Bacillus strain composition according to the methods provided herein can increase production of short chain polysaccharides, monosaccharides, short chain polypeptides, and/or amino acids and/or can increase inhibition of one or more pathogens by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to the absence of the Bacillus strain composition. The production of short chain polysaccharides, monosaccharides, short chain polypeptides, or amino acids can be measured by standard methods including gas chromatography, mass spectrometry, colorimetric assays, and tracer assays. The pathogen inhibitory activity can be measured by standard methods, such as measuring CFUs or number of bacteria in a sample with and without administration of the Bacillus strain composition.

Methods are provided herein for increasing production of short chain polysaccharides, monosaccharides, short chain polypeptides, and/or amino acids in the gastrointestinal tract of a subject. By “subject” as used herein is intended mammals, such as farm animals that can benefit from methods provided herein. In specific embodiments, subjects are poultry (e.g., chickens, broilers, ducks, geese), cows, water buffalos, sheep, goats, pigs, turkeys, horses, donkeys, antelope, deer, elk, camel, llama, aplaca, rabbit, guinea pigs, rodents (e.g., mice, rats, ferret), dogs, cats, monkeys, or apes.

The methods can comprise feeding an animal an effective amount of a Bacillus strain composition provided herein, comprising, e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof, or a combination of any thereof, a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; or a combination of Bacillus subtilis 747 or an active variant thereof and Bacillus subtilis 750 or an active variant thereof. In specific embodiments, the Bacillus strain composition comprises a bacterial cell, a spore, a forespore, and/or a combination of cells, spores, and/or forespores of Bacillus subtilis 747 or an active variant thereof, and a bacterial cell, a spore, a forespore, and/or a combination of cells, spores, and/or forespores of Bacillus subtilis 750 or an active variant thereof. The animal can be fed one or more polysaccharides (e.g., arabinoxylan, cellulose, starch) and/or one or more proteins (e.g., zein, soy protein), e.g., simultaneously or sequentially with the effective amount of the Bacillus strain composition. For example, the animal can be fed feed, forage, or fodder comprising said one or more polysaccharides and/or said one or more proteins. Additionally or alternatively, the Bacillus strain composition can further comprise one or more polysaccharides and/or one or more proteins. In some embodiments, said Bacillus strain composition comprises feed, forage, or fodder comprising said one or more polysaccharides and/or said one or more proteins. In some embodiments, Bacillus strain composition comprises one or more polysaccharides; one or more amino acids; or feed, forage, or fodder comprising one or more polysaccharides or one or more proteins, and further comprises one or more enzymes that catalyzes conversion of the one or more polysaccharides into short chain polysaccharides and/or monosaccharides (e.g., xylanase, arabinoxylanase, amylase, cellulose) or one or more enzymes that catalyzes conversion of the one or more proteins into one or more short chain polypeptides and/or amino acids (e.g., zein protease, soy protease). The one or more enzymes (e.g., xylanase, arabinoxylanase, amylase, cellulose, zein protease, soy protease) can be exogenously added to the one or more polysaccharides, one or more amino acids, feed, forage, fodder, or the Bacillus strain composition to catalyze conversion of the one or more polysaccharides (e.g., xylan, arabinoxylan, starch, cellulose) or one or more proteins (e.g., zein, soy protein).

The effective amount of the Bacillus strain composition can increase production of short chain polysaccharides, monosaccharides, short chain polypeptides, and/or amino acids, and/or can inhibit one or more pathogens (e.g., Escherichia coli, Clostridium, Salmonella, and Streptococcus) in the gastrointestinal tract of an animal. For example, feeding an animal the Bacillus strain composition according to the methods provided herein can increase production of short chain polysaccharides, monosaccharides, short chain polypeptides, and/or amino acids and/or inhibition of one or more pathogens in the gastrointestinal tract of the animal by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a control animal. As used herein, a proper control includes but is not limited to an animal which is not fed the Bacillus strain composition provided herein, an animal which is fed a control composition not comprising the Bacillus strains, or an animal prior to administration of the Bacillus strain composition. One of skill in the art would be able to identify proper controls in order to measure a change (e.g., an increase) in the levels of short chain polysaccharides, monosaccharides, short chain polypeptides, or amino acids. The production of short chain polysaccharides, monosaccharides, short chain polypeptides, or amino acids can be measured by standard methods including gas chromatography, mass spectrometry, colorimetric assays, and tracer assays. The pathogen inhibitory activity can be measured by standard methods, such as measuring CFUs or number of bacteria in a sample with and without administration of the Bacillus strain composition.

The effective amount of the Bacillus strain composition can increase digestibility of one or more polysaccharides (e.g., arabinoxylan, cellulose, starch) or proteins (e.g., zein, soy protein) in the animal. For example, feeding an animal the Bacillus strain composition according to the methods provided herein can increase digestibility of one or more polysaccharides or proteins in the animal by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a control animal, e.g., an animal which is not fed the Bacillus strain composition provided herein, an animal which is fed a control composition not comprising the Bacillus strains, or an animal prior to administration of the Bacillus strain composition. Nutrient digestibility can be measured using any standard methods, such as the total collection of excreta during a period of time, or with the use of an inert marker that is not absorbed by the animal as provided in the present disclosure.

The methods provided herein can be used in any farm having any number of animals (e.g., broilers, chickens), such as 1, 5, 10, 50, 100, 200, 300, 400, 500, 600, 700, 800, 900, 1000, 1500, or more animals. The methods provided herein can be used in a farm having two or more types of animals, such as broilers and turkeys, or chickens and pigs, by feeding respective animals an effective amount of the Bacillus strain composition.

In some embodiments, the effective amount of the Bacillus strain composition for increasing production of short chain polysaccharides, monosaccharides, short chain polypeptides, and/or amino acids, or increasing digestibility of one or more polysaccharides (e.g., arabinoxylan, cellulose, starch) and/or proteins (e.g., zein, soy protein) and/or inhibiting one or more pathogens according to the methods provided herein comprises the amount of the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof, or a combination of any thereof, a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; or a combination of Bacillus subtilis 747 or an active variant thereof and Bacillus subtilis 750 or an active variant thereof) that is at least about 10⁴ CFU to about 10¹² CFU, about 10⁵ CFU to about 10¹² CFU, about 10⁶ CFU to about 10¹² CFU, about 10⁷ CFU to about 10¹² CFU, about 10⁸ CFU to about 10¹² CFU, about 10⁹ CFU to about 10¹² CFU, about 10¹⁰ CFU to about 10¹² CFU, about 10¹¹ CFU to about 10¹² CFU, about 10⁵ CFU to about 10¹¹ CFU, about 10⁵ CFU to about 10¹¹ CFU, about 10⁶ CFU to about 10¹¹ CFU, about 10⁷ CFU to about 10¹¹ CFU, about 10⁸ CFU to about 10¹¹ CFU, about 10⁹ CFU to about 10¹¹ CFU, about 10¹⁰ CFU to about 10¹¹ CFU, about 10⁵ CFU to about 10¹⁰ CFU, about 10⁶ CFU to about 10¹⁰ CFU, about 10⁷ CFU to about 10¹⁰ CFU, about 10⁸ CFU to about 10¹⁰ CFU, or about 10⁹ CFU to about 10¹⁰ CFU. In other embodiments, the effective amount comprises the amount of the Bacillus strain(s) that is at least about 10⁴ CFU, at least about 10⁵ CFU, at least about 10⁶ CFU, at least about 10⁷ CFU, at least about 10⁸ CFU, at least about 10⁹ CFU, at least about 10¹⁰ CFU, at least about 10¹¹ CFU, or at least about 10¹² CFU. In some embodiments, the effective amount of the Bacillus strain composition for increasing production of short chain polysaccharides, monosaccharides, short chain polypeptides, and/or amino acids, or increasing digestibility of one or more polysaccharides (e.g., arabinoxylan, cellulose, starch) and/or proteins (e.g., zein, soy protein), and/or inhibiting one or more pathogens according to the methods provided herein comprises the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof, or a combination of any thereof; a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; or a combination of Bacillus subtilis 747 or an active variant thereof and Bacillus subtilis 750 or an active variant thereof) at a concentration of about 10⁴ CFU/g to about 10¹⁰ CFU/g, about 10⁴ CFU/g to about 10¹¹ CFU/g, at least about 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 10⁵ CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, about 10⁹ CFU/g to about 10¹⁰ CFU/g, about 10⁵ CFU/g to about 10⁹ CFU/g, about 10⁵ CFU/g to about 10⁸ CFU/g, about 10⁵ CFU/g to about 10⁷ CFU/g, about 10⁵ CFU/g to about 10⁶ CFU/g, at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, at least about 10¹² CFU/g, about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, about 10⁹ CFU/ml to about 10¹⁰ CFU/ml, about 10⁵ CFU/ml to about 10⁹ CFU/ml, about 10⁵ CFU/ml to about 10⁸ CFU/ml, about 10⁵ CFU/ml to about 10⁷ CFU/ml, or about 10⁵ CFU/ml to about 10⁶ CFU/ml, at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml. In specific embodiments, the effective amount of the Bacillus composition used in the methods provided herein comprises the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, or Bacillus subtilis 2018, or an active variant thereof; a combination of Bacillus subtilis 750 or active variant thereof and Bacillus subtilis 747 or active variant thereof) at a concentration that does not occur in nature (e.g., at a higher concentration than that found in nature), and using such non-naturally occurring effective amount of the Bacillus strains in the methods provided herein enables (i) increased conversion of one or more polysaccharides into short chain polysaccharides and/or monosaccharides and/or convert one or more proteins into short chain polypeptides and/or amino acids, and/or (ii) increased inhibition of growth of one or more pathogens (e.g., Escherichia coli, Clostridium, Salmonella, and Streptococcus) as compared to the absence of the non-naturally occurring effective amount of the Bacillus strains. In specific embodiments, the effective amount of the Bacillus strain composition comprises the bacterial strain(s) in a non-naturally occurring concentration of about 1.5×10⁵ CFU/gram of composition to about 1.5×10⁶ CFU/gram of composition. In further embodiments, the effective amount of the Bacillus strain composition comprises Bacillus subtilis 750 or an active variant thereof in a non-naturally occurring concentration of about 1.5×10⁵ CFU/gram of composition to about 1.5×10⁶ CFU/gram of composition. In further embodiments, the effective amount of the Bacillus strain composition comprises (i) Bacillus subtilis 750 or an active variant thereof and (ii) at least one of Bacillus subtilis 747, Bacillus subtilis 839, Bacillus subtilis 1781, Bacillus subtilis 1999, or Bacillus subtilis 2018, or an active variant thereof, with optionally (i) and (ii) in equal proportions, in a total concentration of about 1.5×10⁵ CFU/gram of composition to about 1.5×10⁶ CFU/gram of composition, which is a non-naturally occurring concentration. In further specific embodiments, the effective amount of the Bacillus strain composition comprises Bacillus subtilis 747 and Bacillus subtilis 750, optionally in equal proportions, in a total concentration of about 1.5×10⁵ CFU/gram of composition to about 1.5×10⁶ CFU/gram of composition, which is a non-naturally occurring concentration.

In some embodiments, the effective amount of the Bacillus strain composition comprising feed, forage, or fodder (collectively “food”) comprising one or more polysaccharides or proteins for (i) increasing production of short chain polysaccharides, monosaccharides, short chain polypeptides, and/or amino acids, (ii) increasing digestibility of one or more polysaccharides (e.g., arabinoxylan, cellulose, starch) and/or proteins (e.g., zein, soy protein), and/or (iii) increasing according to the methods provided herein comprises the Bacillus strain(s) (e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, Bacillus subtilis 2018, or an active variant thereof, or a combination of any thereof; a combination of i) Bacillus subtilis 750 or an active variant thereof and (ii) at least one of Bacillus subtilis 747, Bacillus subtilis 839, Bacillus subtilis 1781, Bacillus subtilis 1999, or Bacillus subtilis 2018, or an active variant thereof; a combination of Bacillus subtilis 747 or an active variant thereof and Bacillus subtilis 750 or an active variant thereof) at a total concentration of at least about 10⁴ CFU/gram of food to about 10¹² CFU/gram of food, about 10⁵ CFU/gram of food to about 10¹² CFU/gram of food, about 10⁶ CFU/gram of food to about 10¹² CFU/gram of food, about 10⁷ CFU/gram of food to about 10¹² CFU/gram of food, about 10⁸ CFU/gram of food to about 10¹² CFU/gram of food, about 10⁹ CFU/gram of food to about 10¹² CFU/gram of food, about 10¹⁰ CFU/gram of food to about 10¹² CFU/gram of food, about 10¹¹ CFU/gram of food to about 10¹² CFU/gram of food, about 10⁵ CFU/gram of food to about 10¹¹ CFU/gram of food, about 10⁵ CFU/gram of food to about 10¹¹ CFU/gram of food, about 10⁶ CFU/gram of food to about 10¹¹ CFU/gram of food, about 10⁷ CFU/gram of food to about 10¹¹ CFU/gram of food, about 10⁸ CFU/gram of food to about 10¹¹ CFU/gram of food, about 10⁹ CFU/gram of food to about 10¹¹ CFU/gram of food, about 10¹⁰ CFU/gram of food to about 10¹¹ CFU/gram of food, about 10⁵ CFU/gram of food to about 10¹⁰ CFU/gram of food, about 10⁶ CFU/gram of food to about 10¹⁰ CFU/gram of food, about 10⁷ CFU/gram of food to about 10¹⁰ CFU/gram of food, about 10⁸ CFU/gram of food to about 10¹⁰ CFU/gram of food, about 10⁹ CFU/gram of food to about 10¹⁰ CFU/gram of food, about 10⁵ CFU/gram of food to about 10⁹ CFU/gram of food, about 10⁵ CFU/gram of food to about 10⁸ CFU/gram of food, about 10⁵ CFU/gram of food to about 10⁷ CFU/gram of food, about 10⁵ CFU/gram of food to about 10⁶ CFU/gram of food, e.g., at least about 10⁴ CFU/gram of food, at least about 10⁵ CFU/gram of food, at least about 10⁶ CFU/gram of food, at least about 10⁷ CFU/gram of food, at least about 10⁸ CFU/gram of food, at least about 10⁹ CFU/gram of food, at least about 10¹⁰ CFU/gram of food, at least about 10¹¹ CFU/gram of food, or at least about 10¹² CFU/gram of food. In specific embodiments, the feed, forage, or fodder used in the methods provided herein comprises the Bacillus strain(s) at a concentration that does not occur in nature (e.g., at a higher concentration than that found in nature), and using such non-naturally occurring effective amount of the Bacillus strain(s) in the methods enables (i) increased conversion of one or more polysaccharides into short chain polysaccharides and/or monosaccharides and/or convert one or more proteins into short chain polypeptides and/or amino acids, and/or (ii) increased inhibition of growth of one or more pathogens (e.g., Escherichia coli, Clostridium, Salmonella, and Streptococcus) as compared to feed, forage, or fodder not containing the non-naturally occurring effective amount of the Bacillus strains. In specific embodiments, the effective amount of the Bacillus strain composition herein comprises the bacterial strain(s) in a non-naturally occurring concentration of about 1.5×10⁵ CFU/gram of food to about 1.5×10⁶ CFU/gram of food. In further embodiments, the effective amount of the Bacillus strain composition comprises Bacillus subtilis 750 or an active variant thereof in a non-naturally occurring concentration of about 1.5×10⁵ CFU/gram of composition to about 1.5×10⁶ CFU/gram of composition. In further embodiments, the effective amount of the Bacillus strain composition comprises (i) Bacillus subtilis 750 or an active variant thereof and (ii) at least one of Bacillus subtilis 747, Bacillus subtilis 839, Bacillus subtilis 1781, Bacillus subtilis 1999, or Bacillus subtilis 2018, or an active variant thereof, with optionally (i) and (ii) in equal proportions, in a total concentration of about 1.5×10⁵ CFU/gram of composition to about 1.5×10⁶ CFU/gram of composition, which is a non-naturally occurring concentration. In further specific embodiments, the effective amount of the Bacillus strain composition comprises Bacillus subtilis 747 and Bacillus subtilis 750, optionally in equal proportions, in a total concentration of about 1.5×10⁵ CFU/gram of composition to about 1.5×10⁶ CFU/gram of composition, which is a non-naturally occurring concentration.

In some embodiments, the Bacillus strain composition provided herein comprises a bacterial cell, a spore, a forespore, and/or a combination of cells, spores, and/or forespores of (i) Bacillus subtilis 750 or an active variant thereof and (ii) at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof. The Bacillus strain composition provided herein can comprise a population (e.g., cells, spores, forespores, or a combination of any thereof) of Bacillus subtilis 750 or an active variant thereof (“Bacillus subtilis 750 population”) and a population (e.g., cells, spores, forespores, or a combination of any thereof) of at least one of Bacillus subtilis 747, Bacillus subtilis 839, Bacillus subtilis 1781, Bacillus subtilis 1999, Bacillus subtilis 2018, or an active variant thereof (“second Bacillus subtilis population”) at any proportions (e.g., ratios). For example, the Bacillus strain composition can comprise the Bacillus subtilis 750 population and the second Bacillus population at the ratio of 1% to 99%, 2% to 98%, 5% to 95%, 10% to 90%, 15% to 85%, 20% to 80%, 25% to 75%, 30% to 70%, 35% to 65%, 40% to 60%, 45% to 55%, 50% to 50%, 55% to 45%, 60% to 40%, 65% to 35%, 70% to 30%, 75% to 25%, 80% to 20%, 85% to 15%, 90% to 10%, 95% to 5%, 98% to 2%, or 99% to 1%. In specific embodiments, the Bacillus strain composition comprises the Bacillus subtilis 750 population the second Bacillus population in equal proportions (e.g., 50% to 50%, or within 0, 1, 2, 3, 4, 5, 6, 7, 8, 9, 10, 11, 12, 13, 14, 15, 16, 17, 18, 19, or 20% difference between each other, such that the Bacillus subtilis 750 population and the second Bacillus population are in substantially equal proportions).

In some embodiments, the Bacillus strain composition provided herein comprises a bacterial cell, a spore, a forespore, and/or a combination of cells, spores, and/or forespores of Bacillus subtilis 750 or an active variant thereof and Bacillus subtilis 747 or an active variant thereof. The Bacillus strain composition according to the methods provided herein can comprise a population (e.g., cells, spores, forespores, or a combination of any thereof) of Bacillus subtilis 747 or an active variant thereof (“Bacillus subtilis 747 population”) and a population (e.g., cells, spores, forespores, or a combination of any thereof) of Bacillus subtilis 750 (“Bacillus subtilis 750 population”) at any proportions (e.g., ratios). For example, the Bacillus strain composition can comprise the Bacillus subtilis 747 population and the Bacillus subtilis 750 population at the ratio of 1% to 99%, 2% to 98%, 5% to 95%, 10% to 90%, 15% to 85%, 20% to 80%, 25% to 75%, 30% to 70%, 35% to 65%, 40% to 60%, 45% to 55%, 50% to 50%, 55% to 45%, 60% to 40%, 65% to 35%, 70% to 30%, 75% to 25%, 80% to 20%, 85% to 15%, 90% to 10%, 95% to 5%, 98% to 2%, or 99% to 1%. In specific embodiments, a Bacillus strain composition comprises the Bacillus subtilis 747 population the Bacillus subtilis 750 population in equal proportions (e.g., 50% to 50%, or within 0, 1, 2, 3, 4, 5, 6, 7, 8, 9, 10, 11, 12, 13, 14, 15, 16, 17, 18, 19, or 20% difference between each other, such that the Bacillus subtilis 750 population and the second Bacillus population are in substantially equal proportions).

### A. Formulation, Dosing, Route of Administration

The Bacillus strain composition according to the methods provided herein can comprise Bacillus subtilis 750, Bacillus subtilis 747, Bacillus subtilis 839, Bacillus subtilis 1781, Bacillus subtilis 1999, Bacillus subtilis 2018, and/or an active variant thereof, at least one of which can be a powdered and/or lyophilized strain. Lyophilized Bacillus strains and compositions comprising one or more lyophilized Bacillus strains are non-naturally occurring, and can have significantly increased stability, shelf life, and/or storage duration relative to a non-lyophilized or naturally occurring Bacillus strain counterpart, or a composition comprising the same. When germinated, e.g., in the gastrointestinal tract of an animal, a lyophilized Bacillus strain or a composition comprising the same can have significantly different (e.g., increased) biological function, e.g., increased ability to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus) relative to a non-lyophilized or naturally occurring Bacillus strain or composition counterpart that was stored for the same duration of time.

The Bacillus strain composition according to the methods provided herein can further comprise a cryoprotectant, e.g., skim milk (e.g., 10%), glycerin (e.g., 15%), a natural deep eutectic solvent, ice recrystallization inhibitors (e.g., vinyl alcohol, ethylene glycol), sucrose, trehalose, and sodium glutamate. In specific embodiments, the cryoprotectant is not naturally-occurring (i.e., not found in nature) and/or does not naturally occur with the Bacillus strain such that the bacterial strain composition comprising the cryoprotectant is not naturally occurring. The cryoprotectant that does not naturally occur with the Bacillus strain can improve at least one property of the strain such as stability, activity over time, or solubilization. The Bacillus strain composition comprising a cryoprotectant can be stored at a freezing temperature (e.g., −80° C., −20° C.) (i.e., cryopreservation), allowing for a long storage time, low biological mutation rate, and and/or increased activity to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus) as compared to a control composition not comprising a cryoprotectant.

The Bacillus strain composition according to the methods provided herein can further comprise a preservative, e.g., sulfites (e.g., sodium sulfite, sodium bisulfite, sodium metabisulfite, potassium bisulfite, potassium metabisulfite, sulfur dioxide), benzoates (e.g., sodium benzoate), and nitrites (e.g., sodium nitrite). In specific embodiments, the preservative is not naturally-occurring (i.e., not found in nature) and/or does not naturally occur with the Bacillus strain such that the bacterial strain composition comprising the preservative is not naturally occurring. The preservative that does not naturally occur with the Bacillus strain can improve at least one property of the strain such as stability, activity over time, or solubilization. The Bacillus strain composition comprising a preservative can have or increased activity to (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus) as compared to a control composition not comprising a preservative.

The Bacillus strain composition provided herein can further comprise a carrier, e.g., pharmaceutically acceptable carrier, inert diluents (e.g., sodium and calcium carbonate, sodium and calcium phosphate, and lactose), disintegrating agents (e.g., corn starch, alginic acid), binding agents (e.g., starch, gelatin, cellulose, whey powder, rice hulls), lubricating agents (e.g., magnesium stearate, stearic acid, talc), sweetening agents, flavoring agents, coloring agents, coating agents (e.g., glyceryl monostearate, glyceryl distearate). In specific embodiments, the carrier is not naturally-occurring (i.e., not found in nature) and/or does not naturally occur with the Bacillus strain such that the resulting bacterial strain composition is not naturally occurring. In specific embodiments, a carrier that does not naturally occur with the Bacillus strain improves at least one property of the strain such as recovery, efficacy, physical properties, packaging, or administration.

The Bacillus strain composition according to the methods provided herein (i.e., comprising cells of Bacillus subtilis 750, Bacillus subtilis 747, Bacillus subtilis 839, Bacillus subtilis 1781, Bacillus subtilis 1999, Bacillus subtilis 2018, or an active variant thereof, or spores or forespores or a combination of cells, forespores and/or spores, formed from Bacillus subtilis 750, Bacillus subtilis 747, Bacillus subtilis 839, Bacillus subtilis 1781, Bacillus subtilis 1999, Bacillus subtilis 2018, or an active variant thereof, e.g., Bacillus subtilis 750 or an active variant thereof; at least one of Bacillus subtilis 750, Bacillus subtilis 747, Bacillus subtilis 839, Bacillus subtilis 1781, Bacillus subtilis 1999, Bacillus subtilis 2018, or an active variant thereof, or a combination of any thereof; a combination of (i) Bacillus subtilis 750 or an active variant thereof and (ii) at least one of Bacillus subtilis 747, Bacillus subtilis 839, Bacillus subtilis 1781, Bacillus subtilis 1999, Bacillus subtilis 2018, or an active variant thereof; or Bacillus subtilis 747 or an active variant thereof and Bacillus subtilis 750 or an active variant thereof) can be formulated as feed (e.g., as pellets, mash, crumble, cake, meal); as feed, forage, fodder, food, or drink additives (e.g., as powder, liquid, capsule, gel, paste, cream, tablet); in combinations with food or beverage items; in combination with one or more polysaccharides or proteins; in oils; as a suppository; in lubricants; as sachets; or for other administration routes. In some embodiments, the bacterial strain composition disclosed herein is formulated as a liquid formulation or a solid formulation. When the bacterial strain composition is a solid formulation, it may be formulated as a tablet, a sucking tablet, a chewing tablet, a chewing gum, a capsule, a sachet, a powder, a granule, a pellet, a cell pellet, a dust, a coated particle, a coated tablet, an enterocoated tablet, an enterocoated capsule, a melting strip, or a film. When the bacterial strain composition is a liquid formulation, it may be formulated as an aqueous solution, an oil based liquid product, a gel, a suspension, an emulsion, a slurry, a paste, a cell paste, a cream, an ointment, or syrup.

The Bacillus strain composition can be administered to a subject (e.g., farm animal, poultry, broiler, chicken) based on standard techniques for administration to the particular type of subject and in the environment in which the subject receives the bacterial strain composition. When administered to an animal, the bacterial strain composition may be a solid formulation or a liquid formulation. The Bacillus strain composition can be administered mucosally, e.g., via oral administration (e.g., as a feed, forage, fodder, dietary supplement, or pharmaceutical composition), nasal administration, or rectal administration; or administered parenterally, e.g., subcutaneous administration, cutaneous administration, dermal administration, percutaneous administration, transdermal administration, or any method that allows the Bacillus strains of the invention to come into contact with one or more polysaccharides and/or one or more proteins.

The Bacillus strain composition can be administered to an animal, for example according to the protocol exemplified in the present disclosure or in any suitable administration schedule (e.g., as to frequency, duration, and dosage). The method can comprise administration of multiple doses of the Bacillus strain composition, e.g., by mixed in the feed or drinking water for oral consumption by the animal. The method may comprise administration (e.g., feeding) of 1, 2, 3, 4, 5, 6, 7, 8, 9, 10, 15, 20, 25, 30, 35, 40, or more effective doses of the Bacillus strain composition provided herein. In case of a multiple dose administration, the Bacillus strain composition can be administered to (e.g., fed) an animal in a regular interval, such as daily (once a day), multiple times a day (e.g., twice a day, three times a day), once in two days, once in three days, once in four days, once in five days, once in six days, once in a week, once in two weeks, once in three weeks, once in two months, or in a longer interval. Alternatively, the Bacillus strain composition can be administered to (e.g., fed) an animal in a varied interval, such as daily for 7 days and once in two days thereafter. In some embodiments, doses are administered over the course of 1 day, 2 days, 3 days, 4 days, 5 days, 6 days, 7 days, 10 days, 14 days, 21 days, 30 days, 60 days, 90 days, 100 days, 120 days, 150 days, 180 days, 200 days, 210 days, 240 days, 270 days, 300 days, 330 days, one year, or more than one year. The effective amount or dosage of a Bacillus strain composition may increase or decrease over the course of the administration, e.g., over the course of the growth of the animal. Changes in dosage may result or be needed based on the body weight, animal performance, or nutrition efficiency known in the art and described herein.

The frequency, duration, and dosage of application of multiple doses of the Bacillus strain composition in the methods provided herein can be determined by one skilled in the art so as to maintain animal growth and performance (e.g., increase in body weight) when compared to an appropriate control.

In certain embodiments, a bacterial strain or an active variant thereof disclosed herein is administered to a subject (e.g., farm animal, poultry) that is also administered (simultaneously or sequentially) with an additional beneficial microbe. Additional beneficial microbes, such as those described elsewhere herein, may be combined with a bacterial strain of the invention into a formulated product or the beneficial microbes may be administered separately from (before, during, or after) a bacterial strain of the invention. The bacterial strains or an active variants thereof disclosed herein can also be administered to a subject or to bedding that is also administered (simultaneously or sequentially) with an antimicrobial compound, such as an antibiotic compound.

An additional beneficial component (e.g., microbes, probiotics, antimicrobial, medication) may be combined with a bacterial strain of the invention into a formulated product and used in the methods provided herein. Alternatively, additional formulated component may be combined or mixed with a formulated Bacillus strain of the invention into a composition and administered (e.g., fed). Alternatively, the additional component may be administered at a different time. The additional component can exhibit an additional or synergistic effect with the Bacillus strain and/or Bacillus strain composition provided herein to (i) increase production of short chain polysaccharides, monosaccharides, short chain polypeptides, and/or amino acids and/or (ii) increase inhibition of pathogens (e.g., Escherichia coli, Clostridium, Salmonella, and Streptococcus) in the subject. Alternatively or additionally, the additional component can exhibit an additional health promoting effect to animals.

In the embodiments wherein an additional component is administered separately from a bacterial strain of the invention, the bacterial strain composition may be administered before, during, or after the additional component. The bacterial strain of the invention and the additional component (e.g., beneficial microbe, prebiotic, antibiotic) can be administered to an animal within minutes (e.g., 1, 2, 5, 10, 15, 30, 45 minutes), hours (e.g., 1, 2, 3, 4, 5, 6, 7, 8, 9, 10, 12, 15, 20 hours), or days (e.g., 1, 2, 3, 4, 5, 6, 7 days) of each other.

B. Outcomes Associated with the Methods

Methods provided herein that can (i) convert one or more polysaccharides into short chain polysaccharides and/or monosaccharides and/or convert one or more proteins into short chain polypeptides and/or amino acids, and/or (ii) inhibit growth of one or more pathogens (e.g., Escherichia coli, Clostridium, Salmonella, and Streptococcus) can have associated outcomes.

In some embodiments, the method reduces the amount of apparent metabolizable energy (AME) required to maintain performance (e.g., feed conversion ratio, feed efficiency) and/or body weight of the animal, and/or increases nutrition efficiency (i.e., energy efficiency, feed efficiency) in an animal. For example, the methods can decrease the amount of apparent metabolizable energy (AME) required to maintain performance and/or body weight of the animal increases nutrition utilization efficiency in the animal by about 1-20%, 5-20%, 10-20%, 15-20%, more than 20% (e.g., by about 1-2%, 2-5%, 1-5%, 5-10%, 10-15%, 15-20%, more than 20%), e.g., by about 1%, 2%, 3%, 4%, 5%, 6%, 7%, 8%, 9%, 10%, 10%, 15%, 20%, or more than 20%, as compared to a control animal. For example, the method can reduce the amount of daily AME intake required to maintain performance and/or body weight of the animal by about 10-2000 kcal, 10-50 kcal, 50-100 kcal, 100-1000 kcal, 1000-2000 kcal, more than 2000 kcal, e.g., at least about 10 kcal, 20 kcal, 30 kcal, 40 kcal, 50 kcal, 60 kcal, 70 kcal, 80 kcal, 90 kcal, 100 kcal, 200 kcal, 300 kcal, 400 kcal, 500 kcal, 600 kcal, 700 kcal, 800 kcal, 900 kcal, 1000 kcal, 2000 kcal, or more than 2000 kcal in the animal as compared to a control animal. In specific embodiments, the animal is a broiler, and the amount of daily AME intake required to maintain performance and/or body weight is reduced by about 40-80 kcal/kg diet, about 50-75 kcal/kg diet, about 50 kcal/kg diet, or about 75 kcal/kg diet by the methods provided herein. As used herein, a proper control animal includes but is not limited to an animal which is not fed the Bacillus strain composition provided herein, an animal which is fed a control composition not comprising the Bacillus strains, or an animal prior to administration of the Bacillus strain composition. One of skill in the art would be able to identify proper controls in order to assess performance, body weight, or nutrition efficiency of an animal. Reduction of AME intake required to maintain performance and/or body weight in an animal can be assessed by assessing performance and/or body weight of animals that are fed reduced energy diet in which energy is reduced by a certain amount relative to normal diet, and the animals that are fed normal diet. Energy contained in diet can refer to digestible energy or metabolizable energy in the diet by a control animal. Performance of an animal can be measured by standard methods, including assessing average daily gain (ADG), weight, number of eggs produced, scours, mortality, feed conversion (feed/gain), feed efficiency (gain/feed), and feed intake.

In some embodiments, the method increases absorption of nutrients in the gastrointestinal tract of the animal. For example, the method can increase absorption of nutrients in the gastrointestinal tract of the animal by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a control animal (e.g., an animal which is not fed the Bacillus strain composition, an animal which is fed a control composition not comprising the Bacillus strains, an animal prior to administration of the Bacillus strain composition). Absorption of nutrients can be measured by any standard methods, including measuring feed digestibility, e.g., tracer methods (e.g., using one, two, or more types of tracers) to measure amino acids or monosaccharides in the blood and/or excretions.

In some embodiments, the method reduces availability of nutrients in the hindgut of the animal for fermentation and shifts the primary site of nutrient digestion and absorption to the upper intestine (foregut). “Hindgut” as used herein refers to the digestive organs that follow small intestine, including the large intestine and cecum. “Hindgut fermentation” as used herein refers to a digestive process by which nutrients, such as polysaccharides (e.g., cellulose) and proteins can be digested with the aid of bacteria. In animals having energy-deficient diet (e.g., having food/energy intake that is less than what is required to maintain performance or body weight), administering the Bacillus strain composition according to the method provided herein can reduce availability of nutrients in the hindgut for fermentation by about 5-100%, 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 100%, as compared to a control animal (e.g., an animal having energy-deficient diet without or prior to administration of the Bacillus strain composition). Nutrient availability in the hindgut and the extent of hindgut fermentation can be measured by the standard methods. For example, nutrient availability in the hindgut can be measured as the ratio of nutrients in ileal excreta to the ingested nutrients. The extent of hindgut fermentation can be assessed by measuring serum D-lactate levels, with an increased D-lactate level being indicative of increased hindgut fermentation.

In some embodiments, the method increases integrity of a gastrointestinal barrier (gut barrier) in the animal. Without wishing to be bound by theory, a functional gastrointestinal barrier allows absorption of nutrients and fluids, and prevents harmful substances such as toxins and bacteria from crossing the intestinal epithelium into the body. Maintenance of the intestinal barrier is critically important as changes in intestinal permeability allow pathogens to cross into the underlying tissue resulting in inflammation, as well as reduced nutrient absorption and impaired feed conversion (Meyer, F. et al. 2019 Veterinary Res. DOI: 10.1186/s13567-019-0663-x). Impairment of gastrointestinal barrier, or increased gastrointestinal permeability, can be associated with acute and chronic infection, inflammation, morbidity, and mortality in animals. An intact intestinal barrier is the first line of defense in the immune system. A disruption in the intestinal barrier is commonly referred to as leaky gut and is largely a result of dysbiosis which can be caused by stress. Stress in the animal can occur for many reasons including prolonged exposure to high heat, infection, overcrowding, and changes in the diet (Nelson, J. et al. 2018 Animals Doi: 10.3390/ani8100173). Removal of a dietary energy source such as fat can also cause increased stress in the animal leading to dysbiosis.

Probiotics can play an important role in the mediation of intestinal dysbiosis by increasing barrier function and reducing the leakiness of the gut. For example, the beneficial effects of Bacillus subtilis 747, Bacillus subtilis 839, Bacillus subtilis 1781, Bacillus subtilis 1999, or Bacillus subtilis 2018 correlates with intestinal barrier function. Broiler chickens fed a diet containing probiotic Bacillus subtilis 747 and/or Bacillus subtilis 1781 showed a significant increase in gene expression of intestinal barrier (tight junction) proteins ZO1, JAM2, and/or occludin compared to control diet fed broiler chickens. In addition, the broilers fed Bacillus subtilis 747 also showed a significant reduction in feed conversion over a 14-day period. Positive effects on barrier function were shown in Eimeria maxima challenged broiler chickens supplemented with Bacillus subtilis 747 in the diet.

Administration of the Bacillus strain composition (e.g., comprising at least one of Bacillus subtilis 747, Bacillus subtilis 839, Bacillus subtilis 1781, Bacillus subtilis 1999, Bacillus subtilis 2018, or an active variant thereof) according to the method can increase the gastrointestinal barrier integrity by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a control animal (e.g., an animal which is not fed the Bacillus strain composition, an animal which is fed a control composition not comprising the Bacillus strains, an animal prior to administration of the Bacillus strain composition). Gastrointestinal barrier integrity and/or gastrointestinal permeability can be measured by standard methods, such as measuring mRNA or protein levels of tight junction proteins (e.g., occludin, claudin, integral membrane bound cadherins, a-catenin, β-catenin, JAM1, JAM2, ZO-1, ZO-2, ZO-3) by, e.g., PCR, qPCR, Western blot, ELISA; freeze-fracture electron microscopy of transmembrane fibrils; immunostaining of tight junction proteins; measuring the transepithelial resistance (TER; the ability for passive diffusion of ionic charge across the epithelia) or transepithelial electrical resistance (TEER); or measuring passage of solutes over the epithelium via different passage routes, e.g., by using tracer compounds. TEER is the measurement of electrical resistance across a cellular monolayer that reflects the integrity and permeability of the monolayer, and can be measured for example by Ohm's law method or impedance spectroscopy. TEER reflects the ionic conductance of the paracellular pathway in the epithelial monolayer. On the other hand, the flux of non-electrolyte tracers, expressed as permeability coefficient, indicates the paracellular water flow, as well as the pore size of the tight junctions.

In some embodiments, the method enhances mucosal immunity. For example, the method increases the level of secretory IgA (sIgA) in the animal. For example, the method can increase the level of sIgA in the animal 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., increased by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more relative to a control animal (e.g., an animal which is not fed the Bacillus strain composition, an animal which is fed a control composition not comprising the Bacillus strains, an animal prior to administration of the Bacillus strain composition). The level of sIgA can be measured by any standard methods for measuring immunoglobulin levels in a sample, including but not limited to ELISA, immunoblot, and Western blot.

In some embodiments, the method reduces inflammation in the animal. The reduction or decrease in inflammation may include stimulation of intestinal integrity; reduction of intestinal permeability; improvement of mucin synthesis, secretion, and/or quality; improvement of the maturation and differentiation of the intestinal epithelium; improvement of nutrient absorption; increase of the production of soluble factors that transfer antimicrobial activity; stimulation of, improvement of, or support of resistance to infection; support of cellular or humoral responses against viral or bacterial infection; increased cytotoxicity (both anti-viral and anti-tumor); support of systemic and/or mucosal vaccination responses; increase or support of cellular and/or humoral immunity; increase or support of natural immunity (including neutrophils, phagocytes, macrophages, and natural killer cell activity); increase or support of adaptive T and B cell immunity; stimulation of a helper T cell 1 (Th1) cytokine pattern (increased IL-1, IL-2, IFN-γ, IL-12, TNF-α; human leukocyte antigen-Dr (HLA-Dr) expression); suppression of inflammation or production of systemic and mucosal inflammatory mediators (including cytokines and/or chemokines); reduction of sensitization by reducing total and/or allergen-specific IgE; reduction of the production of allergic cytokines; reduction of a Th2 supporting immunoglobulin profile; and combinations thereof when compared to an appropriate control (e.g., the status of the subject prior to administration of the Bacillus strain composition).

As used herein, an “acute phase protein” refers to a protein concentrations of which in blood plasma either increase (positive acute-phase proteins) or decrease (negative acute-phase proteins) in response to inflammation. Acute phase proteins of the present invention includes alpha-1 acid glycoprotein (α-1-AGP). An acute phase protein can be a proinflammatory or anti-inflammatory cytokine.

As used herein, the term “anti-inflammatory cytokine” refers to a naturally occurring or recombinant protein, analog thereof or fragment thereof that elicits an anti-inflammatory response in a cell that has a receptor for that cytokine. Anti-inflammatory cytokines of the invention can be immunoregulatory molecules that control the proinflammatory cytokine response. Anti-inflammatory cytokines of the invention include interleukin (IL)-1 receptor antagonist, IL-4, IL-10, IL-11, and IL-13, IL-16, IFN-α, TGF-beta, G-CSF.

As used herein, the term “proinflammatory cytokine” refers to an immunoregulatory cytokine that favors inflammation. Proinflammatory cytokines of the invention include IL1-α, IL1-β, TNF-α, IL-2, IL-3, IL-6, IL-7, IL-9, IL-12, IL-17, IL-18, LT, LIF, Oncostatin, or IFN-α, IFN-β, IFN-γ.

In some embodiments, administration of the Bacillus strain composition according to the methods provided herein results in decrease in a proinflammatory cytokine or acute phase protein production, which may decrease or prevent an inflammatory response. In specific embodiments, the method reduces a level of IL-6 and/or α-1-AGP in the animal. As used herein, a decrease in the level of pro-inflammatory cytokine production comprises any statistically significant decrease in the level of pro-inflammatory cytokine production in a subject when compared to an appropriate control. Such decreases can include, for example, at least a 5%, 10%, 20%, 30%, 40%, 50%, 60%, 70%, 80%, 90%, or 100% decrease in the level of proinflammatory cytokines when compared to an appropriate control. For example, the methods can reduce the level of proinflammatory cytokines (e.g., α-1-AGP, IL-6) in the animal or a sample obtained from the animal by about 5-100%, 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 100%, as compared to a control. Methods to assay for cytokine levels are known and include, for example Leng S., et al. (2008) J Gerontol A Biol Sci Med Sci 63 (8): 879-884. Methods to assay for the production of pro-inflammatory cytokines include multiplex bead assay, ELISPOT and flow cytometry. See, e.g., Maecker et al. (2005) BMC Immunology 6:13.

In some embodiments, administration of the Bacillus strain composition results in an increase in anti-inflammatory cytokine production. As used herein, an “increase in” or “increasing” anti-inflammatory cytokine production comprises any statistically significant increase in the anti-inflammatory cytokine level when compared to an appropriate control. Such increases can include, for example, at least a 5%, 10%, 20%, 30%, 40%, 50%, 60%, 70%, 80%, 90%, 100%, 150%, 200% or greater increase in the anti-inflammatory cytokine level when compared to an appropriate control. Such increases can also include, for example, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater increase in the anti-inflammatory cytokine level when compared to an appropriate control. Methods to assay for the level of anti-inflammatory cytokine level, are known. See, for example, Leng S., et al. (2008) J Gerontol A Biol Sci Med Sci 63 (8): 879-884. Methods to assay for the production of anti-inflammatory cytokines include multiplex bead assay, ELISA, ELISPOT, qPCR, and flow cytometry. See, e.g., Maecker et al. (2005) BMC Immunology 6:13.

Inflammatory cytokine production can also be measured by assaying the ratio of anti-inflammatory cytokine production to proinflammatory cytokine production. In specific aspects, the ratio of anti-inflammatory cytokine production to proinflammatory cytokine production is increased by about 1, 2, 3, 4, 5, 6, 7, 8, 9, 10, 20, 30, 40, 50, 60, 70, 80, 90, 100, 300, 600, 900, 1000 fold or greater when compared to an appropriate control. In other aspects, the ratio of anti-inflammatory cytokine production to pro-inflammatory cytokine production is increased by about 1 to 5 fold, about 5 to 10 fold, about 10 to 20 fold, about 20 to 30 fold, about 30 to 40 fold, about 40 fold to 60 fold, about 60 fold to 80 fold, about 80 fold to about 100 fold, about 100 to 200 fold, about 200 fold to 300 fold, about 300 to 400 fold, about 400 to about 500 fold, about 500 to about 500 fold, about 500 fold to about 700 fold, about 700 fold to 800 fold, about 800 fold to about 1000 fold or greater when compared to an appropriate control. Methods to determine the ratio of anti-inflammatory cytokine production to pro-inflammatory cytokine production can be found, for example, Leng S., et al. (2008) J Gerontol A Biol Sci Med Sci 63 (8): 879-884. Methods to assay for the production of cytokines include multiplex bead assay, ELISA, ELISPOT, qPCR, and flow cytometry. See, e.g., Maecker et al. (2005) BMC Immunology 6:13.

In specific embodiments, administration of an effective amount of the bacterial strain composition comprising a bacterial strain of the invention can decrease the expression of a marker of inflammation compared to a proper control. Such decreases can include, for example, at least a 5%, 10%, 20%, 30%, 40%, 50%, 60%, 70%, 80%, 90%, or 100% decrease in the level of expression of a marker of inflammation, as measured by mRNA level, qPCR, protein level, ELISA, LPS expression, or any method known in the art, when compared to an appropriate control. The level of the inflammation marker can be measured as it relates to a proper control. As used herein a proper control includes but is not limited to, the expression level of a marker of inflammation in a corresponding sample from a subject that was not administered the bacterial strain composition comprising a bacterial strain of the invention, the expression level of a marker of inflammation in a sample from a subject prior to administration of the bacterial strain composition comprising a bacterial strain of the invention, or the expression level of a marker of inflammation in a standardized sample from a subject that was not administered the bacterial strain composition comprising a bacterial strain of the invention. One of skill in the art would be able to identify proper controls in order to measure an increase in the expression level of a marker of inflammation in an animal.

The following examples are offered by way of illustration and not by way of limitation.

### EXPERIMENTAL

### Example 1: Identification of Enzyme-Producing Bacillus Strains

This Example describes identification of candidate Bacillus strains that can produce a variety of digestive enzymes, particularly amylase, cellulase, and xylanase among a library of spore-forming Bacillus strains isolated from the farm (human-made) settings. These digestive enzymes breakdown resistant fibers and release carbohydrates that are more readily used by the host animal. This should increase the apparent metabolizable energy (AME) of the feed and reduce the feed conversion rate (FCR) for the animal. Alternatively, by releasing more energy from fibers, it may allow FCR performance to be maintained using poorer quality/cheaper feeds.

Candidate strains for enzyme production were screened for enzyme activity. The best producers were identified, and it was confirmed that the strains do not have detrimental characteristics (toxins, antibiotic resistance genes), and have the desired effect of improving feed conversion in animals.

RAPD profiling with primer RAPD #3 (Power, E. G. 1996, J. Hosp. Infect. 34 (4): 247-65) was performed on the spore-forming Bacillus strains and the strains were organized into a phylogenetic tree using the bioinformatics program, Bionumerics. From this tree, representative isolates to screen for enzyme activity were selected from the clusters representing Bacillus species present on the GRAS list.

The original xylanase screen utilized xylan isolated from Beechwood, which is a rather simple polymer with minimal branching substitutions. Digestion assays of Beechwood xylan was done With Tryptic Soy Agar (TSA) with 2% xylan added. Overnight cultures of candidate Bacillus isolates were grown in Tryptic Soy Broth (TSB) media at 32° C. Plates were spotted with 2 μl of this culture and incubated 24 h at 32° C. After incubation, the distance between the edge of the colony and the edge of the zone of clearing was measured in mm using a digital caliper. More relevant forms of xylan are the highly substituted arabinoxylans found in grains. A colorimetric kit for measuring arabinoxylanase activity utilizing a dye-linked wheat arabinoxylan (Megazyme) was used according to the manufacture's protocol to further measure arabinoxylanase activity.

The assay relied on changes in the precipitation of a dye stained arabinoxylan polymer in a specific concentration of ethanol due to digestion effecting the precipitation and separation of undigested, high molecular weight polysaccharides compared the still soluble, digested, lower molecular weight polysaccharides, quantified by measuring Absorbance at 590 nm (Abs 590 nm). Cellulase assays were conducted by preparing agar plates with 0.5 g KH₂PO₄, 0.512 g MgSO₄·7H₂O, 2 g gelatin, 10 g cellulose, 15 g agar per 1 liter H₂O. Plates had an opaque appearance to them. Overnight cultures were spotted onto plates and incubated as described above. After 24 h, 2 ml of 1% Congo Red dye was flooded onto plates and allowed to sit 30 minutes. Excess Congo Red dye was removed, and plates rinsed with 5 ml of sterile water. Plates sat additional 1.5 h and Congo Red stained remaining cellulose. The distance between the edge of the colony and the edge of the zone of clearing was measured.

Amylase assays were conducted by preparing TSA plates with 2% starch. Overnight cultures of candidate Bacillus isolates were grown in TSB at 32° C. Plates were spotted by overnight cultures as described above and incubated 24 h at 32° C. Iodine was dripped onto the plates near the colonies. Dark Blue/Black iodine staining of the starch was observed, as well as any potential zones of clearing around the colony. The distance between the edge of the colony and the edge of the zone of clearing was measured.

Soy protein digestion was investigated by preparing TSA agar plates with 2% soy protein and 15% methanol. Candidates were spotted and incubated as described above. After 24 h incubation, zones of clearing were measured.

Zein protein digestion was investigated by preparing TSA agar plates with 0.5% zein. Candidates were spotted and incubated as described above. After 24 h incubation, zones of clearing were measured. As with Soy protein above, activity tended to be more qualitative than quantitative.

Isolate species identity was determined by purifying gDNA using a Roche DNA isolation kit following the manufacturer's protocol. The 16S rRNA gene was amplified using primers 27F and 1942R and sequenced by Sanger sequencing. Obtained 16S rRNA sequences were compared to known sequences in the NCBI BLAST database.

Comparison of the RAPD profile of selected isolates revealed that five of the 10 top candidates were from a cluster of related isolates. Eight additional isolates were selected from this cluster to be screened for enzyme activity. Of these eight, seven showed high enzyme activity in all relevant categories. Results for representative isolates that showed strong enzyme production are shown in Table 1.

Bacillus subtilis 750 (Bs750) was shown to be the best enzyme producer among those screened, having all 5 enzymatic activities (i.e., arabinoxylanase, cellulase, amylase, soy, zein) with the strongest xylanase activity, very good cellulase activity, and moderately good amylase activity. Strain Bs750 was identified to be a member of the Bacillus subtilis group by 16S rRNA sequencing. Whole genome sequencing showed no toxin genes or transferable antibiotic resistance genes were present.

TABLE 1
Enzymatic activity of select isolates
Arabinoxylanase activity as measured by Abs 590 nm using Megazyme
colorimetric kit; Cellulase and Amylase activity measured as in mm
between edge of colony and edge of zone of clearing; and Soy and Zein
protein digestion recorded as qualitative presence of a zone of
clearing around edge of colony.

| Enzyme | Arabinoxylanase | Cellulase | Amylase | Soy | zein |
|---|---|---|---|---|---|
| Readout | Abs 590 nm | mm | mm | +/− | +/− |
| Strain | | | | | |
| Bs750 | 0.503 | 8.04 | 1.17 | + | + |
| Bs1781 | 0.386 | 7.50 | 1.35 | + | + |
| Bs2018 | 0.355 | 6.65 | 0.93 | + | + |
| Bs1541 | 0.347 | 6.86 | 0.80 | + | + |
| Bs1104 | 0.317 | 6.18 | 1.11 | + | + |
| Bs2084 | 0.241 | 7.07 | 1.01 | + | ND |
| Bs747 | 0.288 | 5.94 | 0.33 | + | +/− |
| Bs27 | 0.024 | 7.44 | 2.45 | − | ND |
| Bl21 | 0.005 | 0.50 | 0.50 | − | ND |
| Ba842 | 0.002 | 0.50 | 0.00 | − | ND |

Bs refers to Bacillus subtilis. Bl21 and Ba842 refer to Bacillus licheniformis 21 and Bacillus licheniformis 842, respectively.

### Example 2: Effect of the Bacillus Strain Composition on Intestinal Barrier Function

This example describes effects of Bacillus subtilis 747-supplemented feed on intestinal cell barrier function in vitro in E. maxima-challenged broiler chickens.

Caco2 cells, a human colorectal cancer cell line, have multiple morphologies and share characteristics of small bowel enterocytes making them an ideal cell line to understand the interaction of probiotic bacteria and intestinal epithelial cells. The Caco2 cell line is an established model to test barrier function due to their ability to differentiate into a polarized epithelium when grown on tissue culture inserts. Polarization of the cells is driven by a family of proteins referred to as tight junction proteins which denote the upper and lower regions of the cells (Bhat, A. et al. Front. Physiol. DOI: 10.3389/fphys.2018.01942). This family includes proteins such as occludin, claudins, zonula occludins (ZO) and junction adhesion molecules (JAM).

Transepithelial resistance assays (TER) or transepithelial electric resistance assays (TEER) of Caco2 cells can demonstrate a strain's effect on tight junctions in vitro by measuring the electrical resistance across a barrier. A baseline electrical resistance for polarized Caco2 cells is determined prior to beginning an assay, then treatments are added. A disruption effect on the intestinal barrier is denoted by a decrease in electrical resistance while a strengthening effect on the barrier will be denoted by an increase in electrical resistance.

Direct effects of Bacillus subtilis 747 on epithelial barrier function was assessed using the TEER in vitro assay. Caco2 cells were treated with Bacillus subtilis 747 (experimental) or tumor necrosis factor alpha (TNF-α) (control), or received no treatment (control) over a 24-hour treatment period and TEER was measured. The statistics were performed using a repeated measures one-way ANOVA with a Dunnett's Post-hoc test using the statistical analysis program, GraphPad PRISM v9.0.

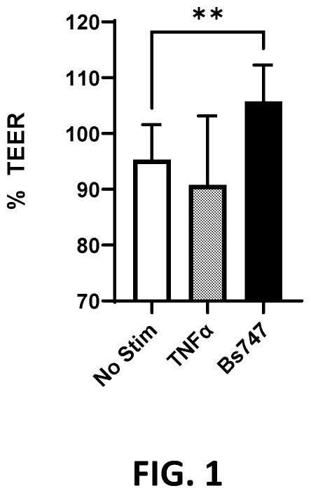

As shown in FIG. 1, Bacillus subtilis 747 demonstrated the ability to significantly increase the TEER over baseline. The significant increase in TEER indicates that Bacillus subtilis 747 is having a positive impact directly on barrier function in vitro. In vivo studies previously demonstrated that broiler chickens fed Bacillus subtilis 747 have significant increase in intestinal tight junction proteins JAM2, occludin, and ZO1 gene expression. Taken together, Bacillus subtilis 747 has the ability to regulate barrier function both in vitro and in vivo. Bacillus subtilis 747-imparted resiliency has the potential to abrogate the effects of dysbiosis caused by stress, including a reduction of energy within the diet, to a commercial bird.

### Example 3: Growth and Digestibility Responses of Broiler Chickens Fed Diets Supplemented with a Bacillus Strain Composition and/or Xylanase

This example describes evaluation of xylanases (enzymes) and Bacillus subtilis 750 (probiotics) on growth and digestibility responses.

The xylanases act by hydrolyzing the arabinoxylans present in the fibrous fraction of the feeds, allowing the endogenous enzymes to reach normally inaccessible sites, increasing the digestibility of nutrients. Additionally, xylanases release some carbohydrates inside the intestine which are taken up by beneficial bacteria, stimulating their growth and maintaining an adequate balance between the gut microbiota and the host. On the other hand, Bacillus subtilis can promote the development of Lactobacillus and to depress the growth of pathogenic bacteria such as E. coli, Clostridium spp, Streptococcus spp and Salmonella spp, leading to improvements in growth and feed conversion.

Effect of a xylanase enzyme and/or a probiotic-supplemented feed on the body weight gain (BWG), feed intake (FI), mortality, feed conversion ratio (FCR), carcass traits, and digestibility of nutrients in broiler chickens were studied.

A growth performance study with broiler chickens from 1 to 42 day of age was carried out at the Poultry Unit of CENIDFyMA-INIFAP. A total of 1, 440 Ross 308 male broiler chickens were allocated in 48, 2x1m floor pens with a bed of wood shavings, in groups of 30 birds per pen. At arrival, chicks were randomly assigned to 1 of 4 treatments, for a total of 12 replicate pens per treatment as follows:

TABLE 2
Treatment Groups

| Treatment | |
|---|---|
| 1 | Control group, was fed a standard corn-soybean meal diet following industry |
| | recommendations, with AGP (Bacitracin methyl salicylate: BMD) and |
| | coccidiostat* |
| 2 | As Control added with xylanase (XYL) |
| 3 | As Control added with probiotic (PRO) |
| 4 | As Control added with XYL and PRO (XYL&PRO) |

*Nicarbazin was used from 1-21 d of age and salinomycin was used from 22-42 d of age as coccidiostats.

Pens were equipped with bell waterers and semi-automatic feeders, in a close unit. The first week the temperature was kept at 32° C., and then, it was reduced 2° C. each week until the third week. Diets were formulated based on ground corn and soybean meal to cover or exceed the nutrient requirements of the broilers according to the recommendations of the Management Guide of the strain (Table 3); all diets were formulated iso-lysinic and iso-caloric to assure the same supply of nutrients among treatments and were fed in mash form. Feed was offered in mash and were divided in 3 phases: starter from 5-14 days of age, grower from 15-28 days of age, and finisher from 29-42 days of age. Broilers had free access to water and feed along the experiment.

The body weight was recorded at 0, 14, 28 and 42 days of age and FI, BWG, FCR and mortality were estimated every two weeks (1-14, 15-28 and 29-42 days) and during the total growth out (1-42 days). At day 28, five birds from each pen were penned in individual cages and were fed with the same diets as before but added with 0.3% of titanium dioxide as inert marker for determination of the ileal digestibility of dry matter, ashes, nitrogen and energy. On days 34 and 35, broilers were killed to get a composite sample; the ileal digesta of five birds from the same pen were pooled to get each replicate. Samples were collected in plastic bags and stored on ice. There were 10 replicate (pooled samples) per treatment. Ileal digesta sample were stored frozen at −20° C. until the lab analysis. At the end of the trial, four broilers per pen were harvested to get the weight and yield of the carcass and main components.

The Ileal digesta samples were lyophilized and ground using a 2 mm mesh. The dry matter, ash, nitrogen, energy and titanium content of the ileal digesta and experimental diets were determined in duplicate. All laboratory determinations were carried out following standard procedures. The ileal apparent digestibility, defined as the ratio of the difference between the ingested nutrient and ileal digesta to the ingested nutrient, of dry matter, ash, nitrogen, and energy were estimated using standard procedures.

For the statistical analysis of results, the procedures of the general lineal models of SAS were used. Data were analyzed using a complete randomized design. For the growth performance there were 12 replicate pens per treatment. For carcass analysis the experimental unit was each broiler with 36 replicate birds per treatment. For the nutrient ileal digestibility, there were 10 replicate samples per treatment and each replicate was the composite sample of the ileal digesta of five broilers of the same treatment. Percentage results were transformed to arcsine values before analysis.

The growth performance variables of broiler chickens fed diets added with a xylanase (XYL) enzyme and a probiotic (PRO) are shown in Table 4. The body weight at 14 days of age and weight gain from 5-14 days of age was lower in broilers fed control diet compared to the other treatments (P<0.01); no differences on 14 days body weight and weight gain were observed among broilers fed xylanase, probiotic, or xylanase and probiotic. On the other hand, the feed conversion ratio (FCR) was higher in broilers fed the control diet compared to the other treatments (P<0.01). No differences on feed conversion were detected among broilers fed xylanase, probiotics, and xylanase with probiotics. The mortality percentage was similar among treatments.

The feed intake and feed conversion ratio from 15-28 days of age were higher in broilers fed the control diet compared to the other treatments (P<0.01); no statistical differences were observed on feed intake and feed conversion among broilers fed xylanase, probiotics, and xylanase with probiotics. However, the weight gain was higher (P<0.05) in broilers fed the control diet and xylanase diets compared to broilers fed the probiotics, and xylanase with probiotics. The mortality percentage was higher in broilers fed the control diet compared to the other treatments (P<0.01), whilst the mortality was similar for xylanase, probiotics, and xylanase with probiotics.

From 29-42 days of age, no differences were observed in any of the productive responses or mortality percentage among treatments.

The feed intake and feed conversion ratio from 5-42 days of age were higher in broilers fed the control diet compared to the other treatments; no differences on feed intake were observed among broilers fed xylanase, probiotics, and xylanase with probiotics. The mortality percentage was higher in broilers fed the control diet compared to the other treatments (P<0.01), whilst the mortality was similar for xylanase, probiotics, and xylanase with probiotics.

The carcass and carcass components weight and yield are shown in Table 5 and the apparent ileal digestibility of nutrients appear in Table 6. No statistical differences were found in any of these variable responses among broilers of the different treatments.

Broiler chickens showed the greatest benefits of the dietary addition of xylanase, probiotics, and xylanase with probiotics during the starter period, and these effects were less during the growing and finishing periods, compared to the control diet broilers. Overall, during the period from 5-42 days of age, xylanase, probiotics, and xylanase with probiotics-treated birds were able to sustain weight gain even though they had lower feed intake, which improved feed conversion. The carcass and carcass components weight and yield were not affected by the dietary addition of xylanase, probiotics, and xylanase with probiotics, but the lower feed conversion indicates that production costs per kg of chicken or meat is lower in the birds that received xylanase, probiotics, and xylanase with probiotics.

The dietary addition of xylanase, probiotics, or xylanase with probiotics did not benefit the apparent ileal digestibility of dry matter, ash, nitrogen or energy. This may suggest that both dietary additives probably acted by stimulating the beneficial microbiota of the intestine and by maintaining adequate intestinal health. Under these conditions, the basal metabolism of the intestine decreases and the demand for immune response supplies is reduced, which translates into a reduction of the nutrient requirements of the GIT and immune functions. Hence, more nutrients are used for growth and meat synthesis.

It is likely that the combination of a xylanase and Bacillus subtilis can potentiate the effects on the growth and intestinal health of broilers chickens compared to the use of each additive separately, which can help to reduce the dependence on the use of antibiotics.

TABLE 3
Experimental starter, grower and finisher diets

| | Starter | | | | Grower | | | | Finisher | | | |
|---|---|---|---|---|---|---|---|---|---|---|---|---|
| | | | | XYL & | | | | XYL & | | | | XYL & |
| Item | CON | XYL | PRO | PRO | CON | XYL | PRO | PRO | CON | XYL | PRO | PRO |
| Ground corn | 50.52 | 49.94 | 49.94 | 49.94 | 64.13 | 63.98 | 63.98 | 63.98 | 66.63 | 66.58 | 66.58 | 66.58 |
| Soybean meal | 41.00 | 41.10 | 41.10 | 41.10 | 29.81 | 29.94 | 29.94 | 29.94 | 26.79 | 26.85 | 26.85 | 26.85 |
| Vegetable oil | 4.22 | 4.30 | 4.30 | 4.30 | 2.33 | 2.40 | 2.40 | 2.40 | 3.27 | 3.31 | 3.31 | 3.31 |
| Ca orthophosphate | 1.70 | 1.70 | 1.70 | 1.70 | 1.23 | 1.23 | 1.23 | 1.23 | 1.00 | 1.00 | 1.00 | 1.00 |
| Ca carbonate | 1.49 | 1.49 | 1.49 | 1.49 | 1.45 | 1.45 | 1.45 | 1.45 | 1.31 | 1.31 | 1.31 | 1.31 |
| Na bicarbonate | 0.20 | 0.20 | 0.20 | 0.20 | 0.20 | 0.20 | 0.20 | 0.20 | 0.20 | 0.20 | 0.20 | 0.20 |
| Salt | 0.32 | 0.32 | 0.32 | 0.32 | 0.30 | 0.30 | 0.30 | 0.30 | 0.28 | 0.28 | 0.28 | 0.28 |
| DL-Methionine | 0.15 | 0.15 | 0.15 | 0.15 | 0.10 | 0.10 | 0.10 | 0.10 | 0.11 | 0.11 | 0.11 | 0.11 |
| L-LysineŸHCl | 0.00 | 0.00 | 0.00 | 0.00 | 0.09 | 0.09 | 0.09 | 0.09 | 0.07 | 0.07 | 0.07 | 0.07 |
| L-Threonine | 0.02 | 0.02 | 0.02 | 0.02 | 0.00 | 0.00 | 0.00 | 0.00 | 0.00 | 0.00 | 0.00 | 0.00 |
| Vit premix | 0.10 | 0.10 | 0.10 | 0.10 | 0.10 | 0.10 | 0.10 | 0.10 | 0.10 | 0.10 | 0.10 | 0.10 |
| Min premix | 0.10 | 0.10 | 0.10 | 0.10 | 0.10 | 0.10 | 0.10 | 0.10 | 0.10 | 0.10 | 0.10 | 0.10 |
| Choline chloride | 0.08 | 0.08 | 0.08 | 0.08 | 0.06 | 0.06 | 0.06 | 0.06 | 0.04 | 0.04 | 0.04 | 0.04 |
| Nicarbazin// | 0.05 | 0.05 | 0.05 | 0.05 | 0.05 | 0.05 | 0.05 | 0.05 | 0.05 | 0.05 | 0.05 | 0.05 |
| Salinomycin | | | | | | | | | | | | |
| BMD | 0.05 | 0.05 | 0.05 | 0.05 | 0.05 | 0.05 | 0.05 | 0.05 | 0.05 | 0.05 | 0.05 | 0.05 |
| XYL&PRO | 0.00 | 0.50 | 0.50 | 0.50 | 0.00 | 0.05 | 0.05 | 0.05 | 0.00 | 0.05 | 0.05 | 0.05 |

Calculated nutrient content

| | | | | | | | | | | | | |
|---|---|---|---|---|---|---|---|---|---|---|---|---|
| ME, kcal/kg | 3000 | 3000 | 3000 | 3000 | 3100 | 3100 | 3100 | 3100 | 3200 | 3200 | 3200 | 3200 |
| CP, % | 23.77 | 23.81 | 23.81 | 23.81 | 19.41 | 19.46 | 19.46 | 19.46 | 18.30 | 18.34 | 18.34 | 18.34 |
| Digestible Lys, % | 1.19 | 1.19 | 1.19 | 1.19 | 1.00 | 1.00 | 1.00 | 1.00 | 0.91 | 0.91 | 0.91 | 0.91 |
| Digestible Met, % | 0.46 | 0.46 | 0.46 | 0.46 | 0.38 | 0.38 | 0.38 | 0.38 | 0.37 | 0.37 | 0.37 | 0.37 |
| Digestible Thr, % | 0.79 | 0.79 | 0.79 | 0.79 | 0.65 | 0.65 | 0.65 | 0.65 | 0.61 | 0.61 | 0.61 | 0.61 |
| Ca, % | 0.90 | 0.90 | 0.90 | 0.90 | 0.95 | 0.95 | 0.95 | 0.95 | 0.85 | 0.85 | 0.85 | 0.85 |
| Available P, % | 0.50 | 0.50 | 0.50 | 0.50 | 0.40 | 0.40 | 0.40 | 0.40 | 0.35 | 0.35 | 0.35 | 0.35 |

TABLE 4
Growth performance of broiler chickens fed diets added
with a xylanase (XYL) enzyme and a probiotic (PRO)

| | | | | |
|---|---|---|---|---|
| CON | XYL | PRO | XYL&PRO | SEM ^{a} |

Growth performance from 5-14 d of age

| | | | | | |
|---|---|---|---|---|---|
| Body weight, 5 d | 93.69 | 93.72 | 95.61 | 94.89 | 1.031 |
| Body weight, 14 d | 420.47 ^{b} | 438.50 ^{c} | 437.77 ^{c} | 436.57 ^{c} | 3.011 |
| Feed intake, g/d | 33.72 | 33.86 | 34.38 | 34.29 | 0.257 |
| Weight gain, g/d | 32.68 ^{b} | 34.48 ^{c} | 34.22 ^{c} | 34.17 ^{c} | 0.311 |
| Feed conversion ratio | 1.03 ^{b} | 0.98 ^{c} | 1.00 ^{c} | 1.00 ^{c} | 0.009 |
| Mortality, % | 1.39 | 0.83 | 0.28 | 0.28 | 0.435 |

Growth performance from 15-28 d of age

| | | | | | |
|---|---|---|---|---|---|
| Body weight, 28 d | 1229.92 | 1249.07 | 1224.96 | 1229.41 | 7.483 |
| Feed intake, g/d | 99.57 ^{b} | 95.46 ^{c} | 94.15 ^{c} | 94.10 ^{c} | 0.658 |
| Weight gain, g/d | 57.82 ^{d} | 57.90 ^{d} | 56.23 ^{e} | 56.63 ^{de} | 0.450 |
| Feed conversion | 1.72 ^{b} | 1.65 ^{c} | 1.67 ^{c} | 1.66 ^{c} | 0.009 |
| Mortality, % | 5.96 ^{b} | 0.29 ^{c} | 0.58 ^{c} | 0.28 ^{c} | 0.726 |

Growth performance from 29-42 d of age

| | | | | | |
|---|---|---|---|---|---|
| Body weight, 42 d | 2453.45 | 2476.38 | 2437.13 | 2446.39 | 18.972 |
| Feed intake, g/d | 152.41 | 148.88 | 149.80 | 148.37 | 1.339 |
| Weight gain, g/d | 87.40 | 87.67 | 86.58 | 86.93 | 1.044 |
| Feed conversion | 1.74 | 1.70 | 1.73 | 1.71 | 0.015 |
| Mortality, % | 0.76 | 0.00 | 0.35 | 1.06 | 0.500 |

Growth performance from 5-42 d of age

| | | | | | |
|---|---|---|---|---|---|
| Feed intake, g/d | 101.71 ^{b} | 98.93 ^{c} | 98.92 ^{c} | 98.35 ^{c} | 0.690 |
| Weight gain, g/d | 63.78 | 64.40 | 63.28 | 63.55 | 0.518 |
| Feed conversion | 1.60 ^{b} | 1.54 ^{c} | 1.56 ^{c} | 1.55 ^{c} | 0.008 |
| Mortality, % | 2.70 ^{b} | 0.37 ^{c} | 0.40 ^{c} | 0.54 ^{c} | 0.554 |

^{a} Standard error of the mean.
^{b-c} Effect of the treatment, P < 0.01.
^{d-e} Effect of the treatment, P < 0.05.

TABLE 5
Carcass and carcass components weight and yield
of broiler chickens fed diets added with a xylanase
(XYL) enzyme and a probiotic (PRO)

| | | | | |
|---|---|---|---|---|
| CON | XYL | PRO | XYL&PRO | SEM ^{a} |

Carcass and carcass components weight, g

| | | | | | |
|---|---|---|---|---|---|
| Legs | 212.26 | 213.99 | 215.10 | 212.46 | 3.234 |
| Thighs | 207.69 | 202.01 | 202.53 | 201.35 | 3.425 |
| Breast | 581.17 | 566.36 | 583.34 | 577.89 | 8.126 |
| Carcass | 1381.24 | 1365.71 | 1381.89 | 1367.61 | 15.440 |

Carcass and carcass components yield, g

| | | | | | |
|---|---|---|---|---|---|
| Legs | 8.53 | 8.73 | 8.79 | 8.62 | 0.116 |
| Thighs | 8.34 | 8.21 | 8.28 | 8.16 | 0.110 |
| Breast | 23.32 | 23.08 | 23.85 | 23.42 | 0.240 |
| Carcass | 55.44 | 55.65 | 56.49 | 55.44 | 0.328 |

^{a} Standard error of the mean.

TABLE 6
Apparent ileal digestibility of broiler chickens fed diets
added with a xylanase (XYL) enzyme and a probiotic (PRO)

| | | | | |
|---|---|---|---|---|
| CON | XYL | PRO | XYL&PRO | SEM ^{a} |

Apparent ileal digestibility, %

| | | | | | |
|---|---|---|---|---|---|
| Dry matter | 68.91 | 69.65 | 69.27 | 68.30 | 0.782 |
| Ashes | 34.46 | 37.25 | 37.91 | 36.76 | 1.047 |
| Nitrogen | 74.68 | 75.48 | 74.52 | 74.30 | 1.786 |
| Energy | 71.88 | 72.30 | 71.79 | 70.93 | 0.833 |

^{a} Standard error of the mean.

### Example 4: Effects of Feed Comprising the Bacillus Strain Composition in Improving Feed Efficiency and Performance on Broilers Fed Reduced Energy Diets, Trial A

This Example tests effects of supplementation of feed with the Bacillus strain compositions on feed efficiency and performance of broiler chickens.

### Methods

The experiment was conducted with male Vencobb 430Y broiler chickens. Day-old chicks were assigned to the four treatment groups with 11 birds/pen and fourteen pens/treatment and had ad libitum access to feed. The first group of broilers were provided (treated) with feed having 50 kcal reduced apparent metabolizable energy (AME) per kg of feed supply (“−50 kcal diet”). The second group of broilers were provided (treated) with the −50 kcal diet supplemented with the Bacillus strain composition comprising Bacillus subtilis 747 and Bacillus subtilis 750 in equal proportions (“Bacillus 747+750 composition”), in a total concentration of Bacillus subtilis 747 plus Bacillus subtilis 750 of about 500 g/ton of feed (i.e., about 1.5×10⁵ CFU/g of feed to about 1.5×10⁶ CFU/g of feed). The third group of broilers were provided (treated) with control feed without energy reduction (“normal energy diet”). The fourth group of broilers were provided (treated) with the normal energy diet supplemented with the Bacillus 747+750 composition. Animal performance (body weight and feed efficiency), nutrient transport, digestibility of dry matter and crude protein, the intestinal site of digestion (upper intestine vs. hindgut), levels of tight junction protein (an indicator of gut barrier integrity), levels of stress indicators, secretory IgA (sIgA) level (an indicator of mucosal immunity) were analyzed. The main effects and interactions are presented and whenever found significant the means were separated by Tukey's B test.

### Results

### Performance:

Effect of the treatments on performance is summarized in Table 7. A decrease in energy by 50 kcal per kg feed showed a tendency for reduced 42-day body weight and increase in feed conversion ratio of broilers. Supplementation with Bacillus strain composition showed a tendency for increased body weight in broilers fed normal diet as well as reduced energy diet, and feed conversion ratio similar to broilers fed the normal energy diet.

TABLE 7
Effect of treatments on broiler performance

| | 42-d | | Feed | |
|---|---|---|---|---|
| | Body | Cumulative | Conversion | European Performance |
| Treatment | Weight | Feed Intake | Ratio | Index (EPI) |
| Normal diet | 2702.7^{ab} | 4094.4 | 1.54^{a} | 407.1 |
| Normal diet + | 2740.3^{b} | 4128.6 | 1.532^{a} | 412.4 |
| Bacillus 747 + 750 composition | | | | |
| Reduced energy diet | 2632.9^{a} | 4070.3 | 1.572^{b} | 388.4 |
| (−50 kcal AME) | | | | |
| Reduced energy diet + Bacillus | 2689.7^{ab} | 4073 | 1.539^{a} | 410.9 |
| 747 + 750 composition | | | | |

Means with dissimilar superscripts in a column varied significantly.

### Physiology:

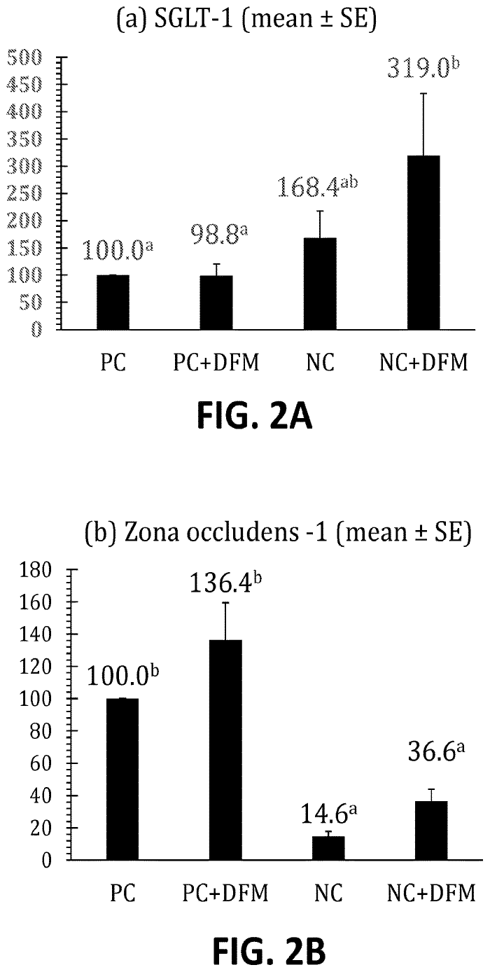

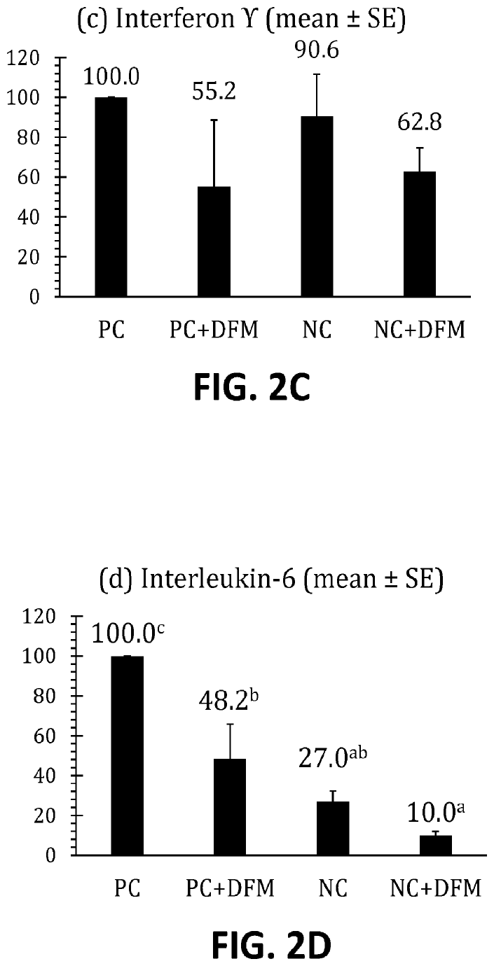

Effects of reduced energy diet and supplementation with the Bacillus 747+750 composition on broiler physiology are summarized in Table 8 and FIG. 2.

A decrease in energy by 50 kcal per kg diet increased levels of acute phase protein Alpha 1 acid glycoprotein (α-1-AGP) (which indicates stress) and supplementation of the reduced energy diet with Bacillus 747+750 composition decreased it at day 42 (Table 8). Broilers fed reduced energy diet had lower expression of gut barrier protein Zona occludens 1 (ZO1) and supplementation with Bacillus 747+750 composition increased its expression (FIG. 2B). Significant main effects were noted for serum D-lactate. Control fed birds had significantly higher levels of D-lactate compared to Bacillus 747+750 composition fed birds (Table 8). Increase in Serum D-lactate can indicate increased hindgut fermentation, dysbacteriosis, poor gut barrier function, and/or malabsorption.

Broilers fed reduced energy diet had decreased mucosal immunity as indicated by level of secretory IgA (sIgA), and lower levels of expression of inflammatory cytokine, interleukin 6 (IL-6), compared to broilers fed normal energy diet (Table 8 and FIG. 2D). Supplementation with Bacillus 747+750 composition in broilers fed normal and reduced energy diet had higher sIgA and a tendency for reduced IL-6 compared to control fed broilers (Table 8 and FIG. 2D). Treatment effects on interferon gamma expression were not statistically significant.

A decrease in energy by 50 kcal per kg diet tended to increase expression of glucose transporter gene SGLT-1 (sodium glucose cotransport protein 1) and supplementation of the reduced energy diet with Bacillus 747+750 composition further increased its expression, presumably improving nutrient assimilation (FIG. 2A).

TABLE 8
Effect of treatments on α -1-AGP (acute phase protein), D-Lactate
(intestinal permeability marker), and secretory IgA (mucosal immunity marker)

| | α -1-AGP μg/mL | | D-lactate nmol/μL | | sIgA μg/mL | |
|---|---|---|---|---|---|---|
| Treatment | 21 day | 42 day | 21 day | 42 day | 21 day | 42 day |
| Normal diet | 132.69 | 216.18^{a} | 0.318^{d} | 0.34^{c} | 3.54^{a} | 8.34^{b} |
| Normal diet + Bacillus | 134.35 | 223.06^{a} | 0.253^{b} | 0.319^{b} | 5.28^{c} | 9.95^{c} |
| 747 + 750 composition | | | | | | |
| Reduced energy diet | 125.51 | 279.09^{b} | 0.28^{c} | 0.323^{b} | 4.29^{ab} | 6.73^{a} |
| (−50 kcal AME) | | | | | | |
| Reduced energy diet + | 128.29 | 221.1^{a} | 0.232^{a} | 0.265^{a} | 4.84^{bc} | 8.2^{b} |
| Bacillus 747 + 750 | | | | | | |
| composition | | | | | | |

Means with dissimilar superscripts in a column varied significantly.

### Ileal Digestibility:

Broilers fed reduced energy diets were noted to have increased nitrogen digestibility. Compared to birds fed normal and reduced energy diet, birds fed diets supplemented with Bacillus 747+750 composition had increased digestibility of dry matter and crude protein (Table 9).

TABLE 9
Effect of treatments on ileal digestibility of nutrients

| | Dry | Organic | | Crude |
|---|---|---|---|---|
| Treatment | matter | matter | Nitrogen | fat |
| Normal diet | 0.704^{a} | 0.752^{a} | 0.678^{a} | 0.787 |
| Normal diet + | 0.763^{b} | 0.751^{a} | 0.711^{b} | 0.792 |
| Bacillus 747 + 750 composition | | | | |
| Reduced energy diet | 0.705^{a} | 0.737^{ab} | 0.711^{b} | 0.777 |
| (−50 kcal AME) | | | | |
| Reduced energy diet + Bacillus | 0.755^{b} | 0.773^{b} | 0.741^{c} | 0.782 |
| 747 + 750 composition | | | | |

Means with dissimilar superscripts in a column varied significantly.

The birds fed the −50 kcal diet exhibited reduced performance, tendency of increased nutrient transport, increased crude protein digestibility, shifting of site of digestion to hindgut for fermentation, decreased gut barrier integrity, a decreased level of sIgA (i.e., decreased mucosal immunity), and increased levels of stress indicators as compared to the birds fed the normal energy diet.

The birds fed the −50 kcal diet supplemented with the Bacillus 747+750 composition exhibited improved performance, tendency to improve nutrient transport, increased digestibility of dry matter and crude protein, reduced nutrients available in the hindgut for fermentation (indicating increased upper intestinal digestion/absorption), increased levels of sIgA (i.e., improved mucosal immunity), and decreased levels of stress indicators as compared to the birds fed the −50 kcal diet energy diet without supplementation with the Bacillus strain composition.

The birds fed the normal energy diet supplemented with the Bacillus 747+750 composition exhibited a tendency for increased body weight, no difference in feed efficiency (and feed conversion ratio), increased digestibility of dry matter and crude protein, reduced availability of nutrients in the hindgut for fermentation, decreased levels of α-1-AGP and/or IL-6 (i.e., improved immunity and reduced inflammation), increased expression of tight junction proteins (i.e., increased gut integrity), and increased levels of sIgA (i.e., improved mucosal immunity) as compared to the birds fed the normal energy diet without supplementation with the Bacillus strain composition.

Similar performance was achieved in birds fed-50 kcal diet supplemented with the Bacillus 747+750 composition relative to birds fed normal energy diet. The data indicate that the improved animal performance is achieved by supplementation of the feed with the Bacillus strain composition, which is driven by better nutrient partitioning (energy partitioning), making more nutrient/energy available for growth. The data further indicate that the improved nutrient/energy partitioning is due to a combination of better nutrient availability and reduced energy spent on immunity and pathogen load, i.e., a combination of improved nutrient digestibility, improved nutrient absorption, improved gut barrier, and improved immunity. Improved gut barrier and improved immunity can be due to shift in gut microbiota and/or reduced subclinical pathogen loads, in addition to reduced nutritional stress due to improved nutrient digestibility and nutrient absorption.

### Example 5: Effects of Feed Comprising the Bacillus Strain Composition in Improving Feed Efficiency and Performance on Broilers Fed Reduced Energy Diets, Trial B

This Example describes effects of supplementation of feed with the Bacillus strain composition in improving feed efficiency and performance of broiler chickens.

### Methods

A total of 350 straight run broiler chicks were divided in 7 groups under different treatments (50 birds/treatment). All the management and vaccine programs were as per the commercially available and applicable broiler farms. The feed was manufactured as per commercial formulations and different treatments were given as starter, grower, and finisher diets. Each group had ad libitum access to feed. Treatment groups and feed formulation (prestarter, starter, finisher) for each treatment groups are shown in Tables 10 and 11-13, respectively. In Table 10, “−50 kcal diet” and “−75 kcal diet” refers to feed having 50 kcal and 75 kcal reduced energy per kg of feed supply, respectively, relative to a normal energy diet. In “1 X dose Bacillus 747+750 composition” groups, the feed was supplemented with the Bacillus strain composition comprising Bacillus subtilis 747 and Bacillus subtilis 750 in equal proportions at 500 g/ton of feed, in a concentration of Bacillus subtilis 747 plus Bacillus subtilis 750 of from about 1.5×10⁵ to about 1.5×10⁶ CFU/g of feed. In “2 X dose Bacillus 747+750 composition” groups, the feed was supplemented with the Bacillus strain composition comprising Bacillus subtilis 747 and Bacillus subtilis 750 in equal proportions at 1 kg/ton of feed, in a concentration of Bacillus subtilis 747 plus Bacillus subtilis 750 of from about 3×10⁵ to about 3×10⁶ CFU/g of feed. Body weight, feed consumption, FCR (pound of feed provided per pound of body weight gain), and mortality were assessed weekly. The results were not statistically analyzed.

TABLE 10
Treatment Groups

| Treatments | Details |
|---|---|
| Control (C) | Control feed having starter, grower, and finisher formulation as per industry |
| | norms |
| T1 | −50 kcal diet + 1 X dose Bacillus 747 + 750 composition (500 g/ton of feed; |
| | about 1.5 × 10⁵-1.5 × 10⁶ CFU/g of feed) |
| T2 | −50 kcal diet + 2 X dose Bacillus 747 + 750 composition (1 kg/ton of feed; about |
| | 3 × 10⁵-3 × 10⁶ CFU/g of feed) |
| T3 | −75 kcal diet + 1 X dose Bacillus 747 + 750 composition (500 g/ton of feed; |
| | about 1.5 × 10⁵-1.5 × 10⁶ CFU/g of feed) |
| T4 | −75 kcal diet + 2 X dose Bacillus 747 + 750 composition (1 kg/ton of feed; about |
| | 3 × 10⁵-3 × 10⁶ CFU/g of feed) |
| T5 | normal energy diet + 1 X dose Bacillus 747 + 750 composition (500 g/ton of |
| | feed; about 1.5 × 10⁵-1.5 × 10⁶ CFU/g of feed) |
| T6 | normal energy diet + 2 X dose Bacillus 747 + 750 composition (1 kg/ton of feed; |
| | about 3 × 10⁵-3 × 10⁶ CFU/g of feed) |

TABLE 11
Feed Formulation (Prestarter)

| PRESTARTER | | | | | | |
|---|---|---|---|---|---|---|
| | T1 | T2 | T3 | T4 | T5 | T6 |
| C | (C-50) + | (C-50) + | (C-75) + | (C-75) + | C + | C + |
| control | 1 X | 2 X | 1 X | 2 X | 1 X | 2 X |

Description

| | | | | | | | |
|---|---|---|---|---|---|---|---|
| MAIZE | 58.51 | 59.77 | 60.4 | 59.77 | 60.4 | 58.51 | 58.51 |
| S B M, 45-47% | 34 | 33.76 | 33.64 | 33.76 | 33.64 | 34 | 34 |
| MBM | 3 | 3 | 3 | 3 | 3 | 3 | 3 |
| vegetable OIL | 1.64 | 0.71 | 0.72 | 0.71 | 0.72 | 1.64 | 1.64 |
| LIMESTONE POWDER | 0.71 | 0.62 | 0.42 | 0.62 | 0.42 | 0.71 | 0.71 |
| DICALCIUM | 0.43 | 0.43 | 0.36 | 0.43 | 0.36 | 0.43 | 0.43 |
| PHOSPHATE | | | | | | | |
| SALT | 0.36 | 0.36 | 0.33 | 0.36 | 0.33 | 0.36 | 0.36 |
| METHIONINE | 0.33 | 0.33 | 0.29 | 0.33 | 0.29 | 0.33 | 0.33 |
| LYSINE | 0.28 | 0.28 | 0.15 | 0.28 | 0.15 | 0.28 | 0.28 |
| CHOLINE CHLORIDE | 0.15 | 0.15 | 0.11 | 0.15 | 0.11 | 0.15 | 0.15 |
| 60% | | | | | | | |
| TOXIN BINDER | 0.1 | 0.1 | 0.1 | 0.1 | 0.1 | 0.1 | 0.1 |
| BETAINE | 0.1 | 0.1 | 0.1 | 0.1 | 0.1 | 0.1 | 0.1 |
| L THREONINE | 0.09 | 0.09 | 0.09 | 0.09 | 0.09 | 0.09 | 0.09 |
| A G P | 0.05 | 0.05 | 0.05 | 0.05 | 0.05 | 0.05 | 0.05 |
| ANTI COCCIDIAL | 0.05 | 0.05 | 0.05 | 0.05 | 0.05 | 0.05 | 0.05 |
| ORGANIC TM | 0.05 | 0.05 | 0.05 | 0.05 | 0.05 | 0.05 | 0.05 |
| VITAMIN PREMIX | 0.03 | 0.03 | 0.03 | 0.03 | 0.03 | 0.03 | 0.03 |
| BROILER | | | | | | | |
| PHYTASE B | 0.01 | 0.01 | 0.01 | 0.01 | 0.01 | 0.01 | 0.01 |
| ANTI OXIDENT | 0.01 | 0.01 | 0.01 | 0.01 | 0.01 | 0.01 | 0.01 |
| XYLANASE | 0.01 | 0.01 | 0.01 | 0.01 | 0.01 | 0.01 | 0.01 |
| | 100 | 100 | 100 | 100 | 100 | 100 | 100 |
| Bacillus 747 + 750 | 0 | 500 g | 1 kg | 500 g | 1 kg | 500 g | 1 kg |
| composition | | | | | | | |
| ME (kcal) | 2,900.00 | 2850 | 2850 | 2825 | 2825 | 2,900.00 | 2,900.00 |
| CF (%) | 3.83 | 3.85 | 3.86 | 3.85 | 3.86 | 3.83 | 3.83 |
| CP (%) | 22 | 22 | 22 | 22 | 22 | 22 | 22 |

TABLE 12
Feed Formulation (Starter)

| STARTER | | | | | | |
|---|---|---|---|---|---|---|
| | T1 | T2 | T3 | T4 | T5 | T6 |
| C | (C-50) + | (C-50) + | (C-75) + | (C-75) + | C + | C + |
| control | 1 X | 2 X | 1 X | 2 X | 1 X | 2 X |

Description

| | | | | | | | |
|---|---|---|---|---|---|---|---|
| MAIZE | 62.78 | 64.03 | 64.66 | 64.03 | 64.66 | 62.78 | 62.78 |
| S B M, 45-47% | 28.84 | 28.59 | 28.47 | 28.59 | 28.47 | 28.84 | 28.84 |
| MBM | 3 | 3 | 3 | 3 | 3 | 3 | 3 |
| VEGETABLE OIL | 2.6 | 1.58 | 1.07 | 1.58 | 1.07 | 2.6 | 2.6 |
| LIMESTONE POWDER | 0.73 | 0.73 | 0.73 | 0.73 | 0.73 | 0.73 | 0.73 |
| DICALCIUM | 0.47 | 0.46 | 0.46 | 0.46 | 0.46 | 0.47 | 0.47 |
| PHOSPHATE | | | | | | | |
| SALT | 0.36 | 0.36 | 0.36 | 0.36 | 0.36 | 0.36 | 0.36 |
| METHIONINE | 0.29 | 0.29 | 0.29 | 0.29 | 0.29 | 0.29 | 0.29 |
| LYSINE | 0.27 | 0.27 | 0.27 | 0.27 | 0.27 | 0.27 | 0.27 |
| CHOLINE CHLORIDE | 0.15 | 0.15 | 0.15 | 0.15 | 0.15 | 0.15 | 0.15 |
| 60% | | | | | | | |
| TOXIN BINDER | 0.1 | 0.1 | 0.1 | 0.1 | 0.1 | 0.1 | 0.1 |
| BETAINE | 0.1 | 0.1 | 0.1 | 0.1 | 0.1 | 0.1 | 0.1 |
| L THREONINE | 0.09 | 0.09 | 0.09 | 0.09 | 0.09 | 0.09 | 0.09 |
| ORGANIC TM | 0.05 | 0.05 | 0.05 | 0.05 | 0.05 | 0.05 | 0.05 |
| A G P | 0.05 | 0.05 | 0.05 | 0.05 | 0.05 | 0.05 | 0.05 |
| ANTI COCCIDIAL | 0.05 | 0.05 | 0.05 | 0.05 | 0.05 | 0.05 | 0.05 |
| VITAMIN PREMIX | 0.03 | 0.03 | 0.03 | 0.03 | 0.03 | 0.03 | 0.03 |
| BROILER | | | | | | | |
| PHYTASE B | 0.01 | 0.01 | 0.01 | 0.01 | 0.01 | 0.01 | 0.01 |
| ANTI OXIDENT | 0.01 | 0.01 | 0.01 | 0.01 | 0.01 | 0.01 | 0.01 |
| XYLANASE | 0.01 | 0.01 | 0.01 | 0.01 | 0.01 | 0.01 | 0.01 |
| | 100 | 100 | 100 | 100 | 100 | 100 | 100 |
| Bacillus 747 + 750 | 0 | 500 g | 1 kg | 500 g | 1 kg | 500 g | 1 kg |
| composition | | | | | | | |
| ME (kcal) | 3,000 | 2950 | 2,950 | 2925 | 2,925 | 3,000 | 3,000 |
| CF (%) | 3.64 | 3.67 | 3.68 | 3.67 | 3.68 | 3.64 | 3.64 |
| CP (%) | 20 | 20 | 20 | 20 | 20 | 20 | 20 |

TABLE 13
Feed Formulation (Finisher)

| FINISHER | | | | | | |
|---|---|---|---|---|---|---|
| | T1 | T2 | T3 | T4 | T5 | T6 |
| C | (C-50) + | (C-50) + | (C-75) + | (C-75) + | C + | C + |
| control | 1 X | 2 X | 1 X | 2 X | 1 X | 2 X |

Description

| | | | | | | | |
|---|---|---|---|---|---|---|---|
| MAIZE | 65.31 | 66.57 | 67.2 | 66.57 | 67.2 | 65.31 | 65.31 |
| S B M, 45-47% | 26.42 | 26.17 | 26.05 | 26.17 | 26.05 | 26.42 | 26.42 |
| MBM | 3 | 3 | 3 | 3 | 3 | 3 | 3 |
| VEGETABLE OIL | 2.96 | 1.94 | 1.43 | 1.94 | 1.43 | 2.96 | 2.96 |
| LIMESTONE POWDER | 0.58 | 0.58 | 0.59 | 0.58 | 0.59 | 0.58 | 0.58 |
| DICALCIUM | 0.36 | 0.36 | 0.36 | 0.36 | 0.36 | 0.36 | 0.36 |
| PHOSPHATE | | | | | | | |
| SALT | 0.31 | 0.3 | 0.3 | 0.3 | 0.3 | 0.31 | 0.31 |
| METHIONINE | 0.24 | 0.24 | 0.24 | 0.24 | 0.24 | 0.24 | 0.24 |
| LYSINE | 0.18 | 0.19 | 0.19 | 0.19 | 0.19 | 0.18 | 0.18 |
| CHOLINE CHLORIDE | 0.15 | 0.15 | 0.15 | 0.15 | 0.15 | 0.15 | 0.15 |
| 60% | | | | | | | |
| TOXIN BINDER | 0.1 | 0.1 | 0.1 | 0.1 | 0.1 | 0.1 | 0.1 |
| BETAINE | 0.1 | 0.1 | 0.1 | 0.1 | 0.1 | 0.1 | 0.1 |
| L THREONINE | 0.06 | 0.06 | 0.06 | 0.06 | 0.06 | 0.06 | 0.06 |
| ORGANIC TM | 0.05 | 0.05 | 0.05 | 0.05 | 0.05 | 0.05 | 0.05 |
| A G P | 0.05 | 0.05 | 0.05 | 0.05 | 0.05 | 0.05 | 0.05 |
| ANTI COCCIDIAL | 0.05 | 0.05 | 0.05 | 0.05 | 0.05 | 0.05 | 0.05 |
| VITAMIN PREMIX | 0.03 | 0.03 | 0.03 | 0.03 | 0.03 | 0.03 | 0.03 |
| BROILER | | | | | | | |
| PHYTASE B | 0.01 | 0.01 | 0.01 | 0.01 | 0.01 | 0.01 | 0.01 |
| ANTI OXIDENT | 0.01 | 0.01 | 0.01 | 0.01 | 0.01 | 0.01 | 0.01 |
| XYLANASE | 0.01 | 0.01 | 0.01 | 0.01 | 0.01 | 0.01 | 0.01 |
| | 100 | 100 | 100 | 100 | 100 | 100 | 100 |
| Bacillus 747 + 750 | 0 | 500 gm | 1 kg | 500 gm | 1 kg | 500 gm | 1 kg |
| composition | | | | | | | |
| ME (kcal) | 3,050 | 3,000 | 3,000 | 2,975 | 2,975 | 3,050 | 3,050 |
| CF (%) | 3.57 | 3.60 | 3.61 | 3.60 | 3.61 | 3.57 | 3.57 |
| CP (%) | 19 | 19 | 19 | 19 | 19 | 19 | 19 |

### Results

As shown in Table 14, body weight was similar among the three groups of broilers each fed the −50 kcal diet with the Bacillus 747+750 composition, the −75 kcal diet with the Bacillus 747+750 composition, and the normal energy diet. Feed efficiency was similar for the birds fed normal energy diet and those fed-50 kcal energy diet supplemented with the Bacillus 747+750 composition, and poorer in birds fed-75 kcal energy diet supplemented with the Bacillus 747+750 composition relative to the other two groups. The data demonstrates that the Bacillus 747+750 composition can maintain performance in broilers fed diets with 50 kcal less energy per kg diet.

TABLE 14
Performance of broilers at 32 days of age

| | | | | | European |
|---|---|---|---|---|---|
| | | | | | Production |
| | | Feed Intake | | | Efficiency |
| Groups | BW (g) | (g) | FCR | Mortality % | Factor (EPEF) |
| Control—C | 1882 | 2863 | 1.521 | 0 | 387 |
| T1 (C − 50) + 1X | 1873 | 2880 | 1.538 | 2% | 381 |
| T2 (C − 50) + 2X | 1877 | 2817 | 1.501 | 0 | 391 |
| T3 (C − 75) + 1 X | 1868 | 2943 | 1.575 | 2% | 371 |
| T4 (C − 75) + 2 X | 1791 | 2857 | 1.595 | 2% | 351 |
| T5 (C) + 1 X | 1814 | 3136 | 1.729 | 4% | 328 |
| T6 (C) + 2 X | 1920 | 2941 | 1.532 | 2% | 392 |

### Example 6: Effects of Feed Comprising the Bacillus Strain Composition in Improving Feed Efficiency and Performance on Broilers Fed Reduced Energy Diets, Trial C

This Example describes effects of supplementation of feed with the Bacillus strain composition in improving feed efficiency and performance of broiler chickens.

### Methods

A total of 300 straight run broiler chick were divided into 3 groups under different treatments and each treatment were run in 4 replicates each. Each treatment group had 25 birds. All the management and vaccine programs were as per commercially available and applicable broiler farms. The feed was manufactured as per commercial formulations and different treatments were given as starter, grower, and finisher diets.

Treatment groups and feed formulations are shown in Tables 15 and 16-18, respectively. The first group of broilers (C) were provided (treated) with control feed without energy reduction (“normal energy diet”). The second group of broilers (T) were provided (treated) with feed having 50 kcal reduced energy per kg of feed supply (“−50 kcal diet”) supplemented with the Bacillus strain composition comprising Bacillus subtilis 747 and Bacillus subtilis 750 in equal proportions (“Bacillus 747+750 composition”) at 0.5 kg/ton of feed, in a concentration of Bacillus subtilis 747 plus Bacillus subtilis 750 of from about 1.5×10⁵ to about 1.5×10⁶ CFU/g of feed. The third group of broilers (NC) were provided (treated) with −50 kcal diet. Each group had ad libitum access to feed. Body weight feed consumption, and FCR (pounds of feed provided/pound of body weight) were assessed weekly. The number of replicates in the study were not sufficient to run statistical analysis.

TABLE 15
Treatment Groups

| Treatment | Details |
|---|---|
| Control—C | Control feed having starter, grower, and finisher |
| | formulation as per industry norms |
| Treatment—T | −50 kcal reduced AME diet + Bacillus 747 + |
| | 750 composition (0.5 kg/ton of feed) |
| Negative Control—NC | −50 kcal reduced AME diet |

TABLE 16
Feed Formulation (Prestarter)

| | PRESTARTER | | |
|---|---|---|---|
| | | T | |
| | C | C − 50 kcal + | NC |
| Description | control | 1X | C − 50 kcal |
| MAIZE | 58.51 | 59.77 | 59.77 |
| S B M, 45-47% | 34 | 33.76 | 33.76 |
| MBM | 3 | 3 | 3 |
| VEGETABLE OIL | 1.64 | 0.71 | 0.71 |
| LIMESTONE | 0.71 | 0.62 | 0.62 |
| POWDER | | | |
| DICALCIUM | 0.43 | 0.43 | 0.43 |
| PHOSPHATE | | | |
| SALT | 0.36 | 0.36 | 0.36 |
| METHIONINE | 0.33 | 0.33 | 0.33 |
| LYSINE | 0.28 | 0.28 | 0.28 |
| CHOLINE | 0.15 | 0.15 | 0.15 |
| CHLORIDE 60% | | | |
| TOXIN BINDER | 0.1 | 0.1 | 0.1 |
| BETAINE | 0.1 | 0.1 | 0.1 |
| L THREONINE | 0.09 | 0.09 | 0.09 |
| A G P | 0.05 | 0.05 | 0.05 |
| ANTI COCCIDIAL | 0.05 | 0.05 | 0.05 |
| ORGANIC TM | 0.05 | 0.05 | 0.05 |
| VITAMIN PREMIX | 0.03 | 0.03 | 0.03 |
| BROILER | | | |
| PHYTASE B | 0.01 | 0.01 | 0.01 |
| ANTI OXIDENT | 0.01 | 0.01 | 0.01 |
| XYLANASE | 0.01 | 0.01 | 0.01 |
| | 100 | 100 | 100 |
| Bacillus 747 + 750 | 0 | 500 g/ton | 0 |
| composition | | | |
| ME (kcal) | 2,900.00 | 2850 | 2850 |
| CF (%) | 3.83 | 3.85 | 3.85 |
| CP (%) | 22 | 22 | 22 |

TABLE 17
Feed Formulation (Starter)

STARTER

| | C | T | NC |
|---|---|---|---|
| Description | control | (C − 50) + 1 X | (C − 50) |
| MAIZE | 62.78 | 64.03 | 64.03 |
| S B M, 45-47% | 28.84 | 28.59 | 28.59 |
| MBM | 3 | 3 | 3 |
| VEGETABLE OIL | 2.6 | 1.58 | 1.58 |
| LIMESTONE | 0.73 | 0.73 | 0.73 |
| POWDER | | | |
| DICALCIUM | 0.47 | 0.46 | 0.46 |
| PHOSPHATE | | | |
| SALT | 0.36 | 0.36 | 0.36 |
| METHIONINE | 0.29 | 0.29 | 0.29 |
| LYSINE | 0.27 | 0.27 | 0.27 |
| CHOLINE | 0.15 | 0.15 | 0.15 |
| CHLORIDE 60% | | | |
| TOXIN BINDER | 0.1 | 0.1 | 0.1 |
| BETAINE | 0.1 | 0.1 | 0.1 |
| L THREONINE | 0.09 | 0.09 | 0.09 |
| ORGANIC TM | 0.05 | 0.05 | 0.05 |
| A G P | 0.05 | 0.05 | 0.05 |
| ANTI COCCIDIAL | 0.05 | 0.05 | 0.05 |
| VITAMIN PREMIX | 0.03 | 0.03 | 0.03 |
| BROILER | | | |
| PHYTASE B | 0.01 | 0.01 | 0.01 |
| ANTI OXIDENT | 0.01 | 0.01 | 0.01 |
| XYLANASE | 0.01 | 0.01 | 0.01 |
| | 100 | 100 | 100 |
| Bacillus 747 + 750 | 0 | 500 g/ton | 0 |
| composition | | | |
| ME (kcal) | 3,000 | 2950 | 2950 |
| CF (%) | 3.64 | 3.67 | 3.67 |
| CP (%) | 20 | 20 | 20 |

TABLE 18
Feed Formulation (Finisher)

FINISHER

| | C | T | NC |
|---|---|---|---|
| Description | control | (C − 50) + 1 X | (C − 50) |
| MAIZE | 65.31 | 66.57 | 66.57 |
| S B M, 45-47% | 26.42 | 26.17 | 26.17 |
| MBM | 3 | 3 | 3 |
| VEGETABLE OIL | 2.96 | 1.94 | 1.94 |
| LIMESTONE | 0.58 | 0.58 | 0.58 |
| POWDER | | | |
| DICALCIUM | 0.36 | 0.36 | 0.36 |
| PHOSPHATE | | | |
| SALT | 0.31 | 0.3 | 0.3 |
| METHIONINE | 0.24 | 0.24 | 0.24 |
| LYSINE | 0.18 | 0.19 | 0.19 |
| CHOLINE | 0.15 | 0.15 | 0.15 |
| CHLORIDE 60% | | | |
| TOXIN BINDER | 0.1 | 0.1 | 0.1 |
| BETAINE | 0.1 | 0.1 | 0.1 |
| L THREONINE | 0.06 | 0.06 | 0.06 |
| ORGANIC TM | 0.05 | 0.05 | 0.05 |
| A G P | 0.05 | 0.05 | 0.05 |
| ANTI COCCIDIAL | 0.05 | 0.05 | 0.05 |
| VITAMIN PREMIX | 0.03 | 0.03 | 0.03 |
| BROILER | | | |
| PHYTASE B | 0.01 | 0.01 | 0.01 |
| ANTI OXIDENT | 0.01 | 0.01 | 0.01 |
| XYLANASE | 0.01 | 0.01 | 0.01 |
| | 100 | 100 | 100 |
| Bacillus 747 + 750 | 0 | 500 g/ton | 0 |
| composition | | | |
| ME (kcal) | 3,050 | 3,000 | 3,000 |
| CF (%) | 3.57 | 3.60 | 3.60 |
| CP (%) | 19 | 19 | 19 |

### Results

As shown in Table 19, energy reduction by 50 kcal decreased broiler body weight and increased mortality adjusted feed conversion ratio (FCR) compared to birds fed diets with normal energy recommended for the breed. Supplementation of the −50 kcal diet with the Bacillus 747+750 composition resulted in similar body weight and adjusted FCR in chickens that were achieved by normal energy diet. The data demonstrates that the Bacillus 747+750 composition can maintain performance in broilers fed diets with 50 kcal less energy per kg feed daily.

TABLE 19
Performance of broilers at 35 days of age

| | Cumulative body | Adjusted |
|---|---|---|
| TREATMENTS | weight gain (g) | FCR |
| Normal energy diet | 1774.25 | 1.681 |
| −50 kcal diet | 1698.61 | 1.698 |
| −50 kcal diet + Bacillus 747 + | 1760.01 | 1.685 |
| 750 composition | | |

### Example 7: Effects of Feed Comprising the Bacillus Strain Composition in Improving Feed Efficiency and Performance on Broilers Fed Reduced Energy Diets, Trial D

This Example describes effects of supplementation of feed with the Bacillus strain composition in improving feed efficiency and performance of broiler chickens.

### Methods

A total of 8,000 straight run broiler chicks were divided into 2 groups under different treatments. Each treatment group had 4,000 birds. All the management and vaccine programs were as per commercially available and applicable broiler farms. The feed was manufactured as per commercial formulations.

The first group of chicks (C) were provided (treated) with control feed without energy reduction (“normal energy diet”). The second group of chicks (T) were provided (treated) with feed having 50 kcal reduced energy per kg of feed supply (“−50 kcal diet”) supplemented with the Bacillus strain composition comprising Bacillus subtilis 747 and Bacillus subtilis 750 in equal proportions (“Bacillus 747+750 composition”) at 0.5 kg/ton of feed, in a concentration of Bacillus subtilis 747 plus Bacillus subtilis 750 of from about 1.5×10⁵ to about 1.5×10⁶ CFU/g of feed. The −50 kcal diet was produced by replacing 7% of the soybean oil in the normal energy diet with corn. Each group had ad libitum access to feed. Body weight feed consumption, and FCR (pounds of feed provided/pound of body weight) were assessed weekly. The number of replicates in the study were not sufficient to run statistical analysis.

### Results

Performance of birds fed diet containing 50 kcal lower energy supplemented with the Bacillus 747+750 composition was better compared to birds fed control diet with normal energy without Bacillus 747+750 composition supplementation. Finishing average body weight was higher by 2.69 kg in the treated birds fed the −50 kcal diet with Bacillus 747+750 composition and by 2.46 kg in the control birds fed the normal energy diet, as compared to the respective average body weight at the beginning of the study. Feed Conversion Ratio (FCR) was better (lower, indicating more efficiency) in the treated birds fed the −50 kcal diet with Bacillus 747+750 composition (1.82) relative to the control birds fed the normal energy diet (2.09).

In conclusion, similar performance was achieved in birds fed 50 kcal lower energy and supplemented with Bacillus subtilis strains 747+750 as birds fed normal energy level. The mechanism of sustained performance in lower energy fed birds appears to be not solely related to the improved feed digestion, but likely a combination of better nutrient availability and reduced energy spent on immunity and pathogen load, caused by supplementation with Bacillus subtilis strains 747 and 750.

Economics: All costs are in Indian Rupees (INR). 1 USD=˜80 INR

The above Examples demonstrate that supplementation of reduced energy feed (e.g.,−50 kcal feed) with select Bacillus subtilis strains (e.g., Bacillus subtilis strains 747 and 750) maintains performance of animals (e.g., birds, broilers) fed reduced energy at the level of counterpart animals fed normal energy level. Reducing energy in feed by 50 kcal/kg via oil reduction (e.g., replacing soybean oil with corn) in all three phases (i.e., prestarter, starter, and finisher feed) can reduce ration cost by about USD $8.0 per ton. If the Bacillus 747+750 composition can be obtained at about USD $2.0-2.5 per ton of feed, there will be a savings for the animal farm business of about USD $5.5-6.0 per ton of feed.

1. A Bacillus strain composition comprising a bacterial cell, a spore, a forespore, and/or a combination of cells, spores, and/or forespores of Bacillus strain Bacillus subtilis 750 or an active variant thereof, wherein an effective amount of said Bacillus strain composition increases conversion of (i) one or more polysaccharides into short chain polysaccharides and/or monosaccharides and/or (ii) one or more proteins into short chain polypeptides and/or amino acids, relative to the absence of said effective amount of said Bacillus strain composition.

2. The Bacillus strain composition of claim 1, further comprising a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof;
comprising said at least one of Bacillus subtilis 747, Bacillus subtilis 839, Bacillus subtilis 1781, Bacillus subtillis 1999, Bacillus subtilis 2018, or an active thereof and said Bacillus subtilis 750 or active thereof in equal proportions,
comprising a bacterial cell, a spore, a forespore, and/or a combination of cells, spores, and/or forespores of said Bacillus subtilis 747 or active variant thereof; or
comprising said Bacillus subtilis 747 or active variant thereof and said Bacillus subtilis 750 or active variant thereof in equal proportions.

3.-5. (canceled)

6. The Bacillus strain composition of claim 1,
wherein said bacterial cell, spore, forespore, and/or combination of cells, spores, and/or forespores of said Bacillus strain(s) is present in the composition at about 1×10⁵ CFU/gram to about 1×10¹⁰ CFU/gram or at about 1×10⁵ CFU/ml to about 1×10¹⁰ CFU/ml;
wherein said bacterial cell, spore, forespore, and/or combination of cells, spores, and/or forespores of said Bacillus subtilis 747 or active variant thereof and said Bacillus subtilis 750 or active variant thereof is present in the composition about 1×10⁵ CFU/gram to about 1×10¹⁰ CFU/gram or at about 1×10⁵ CFU/ml to about 1×10¹⁰ CFU/ml;
wherein at least one of said Bacillus subtilis strains is a powdered, lyophilized strain;
wherein the composition further comprises a cryoprotectant;
wherein the composition further comprises a preservative;
wherein the composition further comprises said one or more polysaccharides and/or said one or more proteins;
said composition comprises feed, forage, or fodder comprising said one or more polysaccharides and/or said one or more proteins.

7.-12. (canceled)

13. A Bacillus strain composition comprising one or more polysaccharides and/or one or more proteins and a Bacillus strain, said Bacillus strain comprising a bacterial cell, a spore, a forespore, and/or a combination of cells, spores, and/or forespores of Bacillus subtilis 750 or an active variant thereof.

14. The Bacillus strain composition of claim 13,
further comprising a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof;
comprising said at least one of Bacillus subtilis 747, Bacillus subtilis 839, Bacillus subtilis 1781, Bacillus subtilis 1999, Bacillus subtilis 2018, or an active variant thereof and said Bacillus subtilis 750 or active variant in equal proportions;
comprising a bacterial cell, a spore, a forespore, and/or a combination of cells, spores, and/or forespores of said Bacillus subtilis 747 or active variant thereof; and/or
comprising said Bacillus subtilis 747 or active variant thereof and said Bacillus subtilis 750 or active var it thereof in equal proportions.

15.-17. (canceled)

18. The Bacillus strain composition of claim 13, wherein said bacterial cell, spore, forespore, and/or combination of cells, spores, and/or forespores of said Bacillus strain(s) is present in the composition at about 1×10⁵ CFU/gram to about 1×10¹⁰ CFU/gram or at about 1×10⁵ CFU/ml to about 1×10¹⁰ CFU/ml;
wherein said bacterial cell, spore, forespore, and/or combination of cells, spores, and/or forespores of said Bacillus subtilis 747 or active variant thereof and said Bacillus subtilis 750 or active variant thereof is present in the about 1×10⁵ CFU/gram to about 1×10¹⁰ CFU/gram or at about 1×10⁵ CFU/ml to about 1×10¹⁰ CFU/ml;
wherein the composition further comprising feed, forage, or fodder comprising said one or more polysaccharides or said one or more proteins;
wherein said bacterial cell, spore, forespore, and/or combination of cells, spores, and/or forespores of said Bacillus strain(s) is present in the composition at about 1.5×10⁵ CFU/gram of feed, forage, or fodder to about 1.5×10⁶ CFU/gram of feed, forage, or fodder;
wherein said bacterial cell, spore, forespore, and/or combination of cells, spores, and/or forespores of said Bacillus subtilis 747 or active variant thereof and said Bacillus subtilis 750 or active variant thereof is present in the composition at about 1.5×10⁵ CFU/gram of feed, forage, or fodder to about 1.5×10⁶ CFU/gram of feed, forage, or fodder;
wherein an effective amount of said Bacillus strain composition increases conversion of (i) one or more polysaccharides into short chain polysaccharides and/or monosaccharides and/or (ii) one or more proteins into short chain polypeptides and/or amino acids relative to the absence of said effective amount of said Bacillus strain composition;
wherein said one or more of polysaccharides comprise one or more non-starch polysaccharides;
wherein said one or more non-starch polysaccharides comprises arabinoxylan and/or cellulose;
wherein said one or more proteins comprise zein and/or soy protein;
wherein said Bacillus strain composition comprises an activity of arabinoxylanase cellulase, amylase, zein protease; and/or soy protease; and/or
wherein said Bacillus strain composition is formulated as pellets, mash, crumble, cake, meal, powder, or liquid.

19.-23. (canceled)

24. The Bacillus strain composition of claim 1, wherein said one or more polysaccharides comprise one or more non-starch polysaccharides:
wherein said one or more non-starch polysaccharides comprise arabinoxylan and/or cellulose;
wherein said one or more proteins comprise zein and/or soy protein;
wherein said Bacillus strain composition comprises an activity of arabinoxylanase, cellulase, zein protease, and/or soy protease;
wherein said Bacillus strain composition is formulated as pellets, mash, crumble, cake, meal, powder or liquid.

25.-28. (canceled)

29. The Bacillus strain composition of claim 1, wherein said Bacillus subtilis 750 is deposited under NRRL accession number B-68212.

30. The Bacillus strain composition of claim 2, wherein said Bacillus subtilis 747 is deposited under NRRL accession number B-67257, said Bacillus subtilis 839 is deposited under NRRL accession number B-67951, said Bacillus subtilis 1781 is deposited under NRRL accession number B-67259, said Bacillus subtilis 1999 is deposited under NRRL accession number B-67318, and/or said Bacillus subtilis 2018 is deposited under NRRL accession number B-67261.

31. A method of increasing production of short chain polysaccharides, monosaccharides, short chain polypeptides, and/or amino acids, said method comprising contacting one or more polysaccharides and/or one or more proteins with an effective amount of a Bacillus strain composition comprising a bacterial cell, a spore, a forespore, and/or a combination of cells, spores, and/or forespores of Bacillus subtilis 750 or an active variant thereof.

32. The method of claim 31, wherein the Bacillus strain composition further comprises a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof;
wherein the Bacillus strain composition comprises at least one of Bacillus subtilis 747, Bacillus subtilis 839, Bacillus subtilis 1781, Bacillus subtilis 1999, Bacillus subtilis 2018, or an active variant thereof and said Bacillus subtilis 750 or active variant thereof in equal proportions;
wherein the Bacillus strain composition comprises a bacterial cell, a spore, a forespore, and/or a combination of cells, spores, and/or forespores of said Bacillus subtilis 747 or active variant thereof;
wherein the Bacillus strain composition comprises said Bacillus subtilis 747 or active variant thereof and said Bacillus subtilis 750 or active variant thereof in equal proportions; and/or
wherein the method comprises contacting feed, forage, or fodder comprising said one or more polysaccharides and/or said one or more proteins with said effective amount of said Bacillus strain composition.

33.-36. (canceled)

37. The method of claim 31, comprising feeding an animal said effective amount of a Bacillus strain composition, wherein the production of short chain polysaccharides, monosaccharides, short chain polypeptides, and/or amino acids is increased in the gastrointestinal tract of said animal.

38. The method of claim 37, wherein the Bacillus strain composition further comprises a bacterial cell, a spore, a forespore, and/or a combination of cells, spores, and/or forespores of at least one Bacillus strain selected from the group consisting of Bacillus subtilis 747, Bacillus subtilis 839, Bacillus subtilis 1781, Bacillus subtilis 1999, Bacillus subtilis 2018, and an active variant thereof;
wherein the Bacillus strain composition comprises at least one of Bacillus subtilis 747, Bacillus subtilis 839, Bacillus subtilis 1781, Bacillus subtilis 1999, Bacillus subtilis 2018, or an active variant thereof and said Bacillus subtilis 750 or active variant thereof in equal proportions;
wherein the Bacillus strain composition comprises a bacterial cell, a spore, a forespore, and/or a combination of cells, spores, and/or forespores of Bacillus subtilis 747 or active variant thereof;
wherein the Bacillus strain composition comprises said Bacillus subtilis 747 or active variant thereof and said Bacillus subtilis 750 or active variant thereof in equal proportions;
wherein said method increases digestibility of one or more polysaccharides and/or one or more proteins in animal;
wherein said animal is fed one or more polysaccharides and/or one or more proteins;
wherein said animal is fed feed, forage, or fodder comprising said one or more polysaccharides and/or said one or more proteins;
wherein Bacillus subtilis strain composition further comprises said one or more polysaccharides and/or said one or more proteins;
wherein said Bacillus strain composition comprises feed, forage, or fodder comprising said one or more polysaccharides and/or said one or more proteins;
wherein said bacterial cell, spore, forespore, and/or combination of cells, spores, and/or forespores of said Bacillus strain(s) is present in said Bacillus strain composition at about 1.5×10⁵ CFU/gram of feed, forage, or fodder to about 1.5×10⁶ CFU/gram of feed, forage, or fodder; and/or
wherein said bacterial cell, spore, forespore, and/or combination of cells, spores, and/or forespores of said Bacillus subtilis 747 or active variant thereof and said Bacillus subtilis 750 or active variant thereof is present in said Bacillus strain composition at about 1.5×10⁵ CFU/gram of feed, forage, or fodder to about 1.5×10⁶ CFU/gram of feed, forage or fodder.

39.-48. (canceled)

49. The method of claim 37, wherein said method:
reduces apparent metabolizable energy (AME) required to maintain performance and/or body weight of the animal;
increases nutrition efficiency in the animal;
increases absorption of nutrients in the gastrointestinal tract of the animal;
reduces availability of nutrients in the hindgut of the animal for fermentation;
increases integrity of a gastrointestinal barrier in the animal;
increases an level of secretory IgA in the animal;
reduces inflammation in the animal;
reduces a level of alpha-1 acid glycoprotein (α-1-AGP) and/or IL-6 in the animal; and/or
inhibits a pathogen in the gastrointestinal tract of the animal; and/or
improves energy partitioning in the animal;
wherein the pathogen is one or more of Escherichia coli, Clostridium, Salmonella, and Streptococcus;
wherein said animal is an avian animal or a swine; and/or
wherein said animal is a chicken, and the method reduces the amount of apparent metabolizable energy required to maintain performance and/or body weight by about 50 kcal per kg diet fed ad libitum to chickens.

50.-52. (canceled)

53. The method of any claim 31, wherein said effective amount comprises at about 1×10⁵ CFU/gram to about 1×10¹⁰ CFU/gram or at about 1×10⁵ CFU/ml to about 1×10¹⁰ CFU/ml of said bacterial cell, spore, forespore, and/or combination of cells, spores, and/or forespores of said Bacillus strain(s);
wherein said effective amount comprises at about 1.5×10⁵ CFU/gram of feed, forage, or fodder to about 1.5×10⁶ CFU/gram of forage, or fodder of said bacterial cell, spore, forespore, and/or combination of cells, spores, forespores of said Bacillus strain(s);
wherein said effective amount comprises at about 1×10⁵ CFU/gram to about 1×10¹⁰ CFU/gram or at about 1×10⁵ CFU/ml to about 1×10¹⁰ CFU/ml of said bacterial cell, spore, forespore, and/or combination of cells, spores, and/or forespores of said Bacillus subtilis 747 or active variant thereof and said Bacillus subtilis 750 or active variant thereof;
wherein said effective amount comprises at about 1.5×10⁵ CFU/gram of feed, forage, or fodder to about 1.5×10⁶ CFU/gram of feed, forage, or fodder of said bacterial cell, spore, forespore, and/or combination of cells, spores, and/or forespores of Bacillus subtilis 747 or active variant thereof and said Bacillus subtilis 750 or active variant thereof;
wherein at leas one of said Bacillus strains is a powdered, lyophilized strain;
wherein said one or more polysaccharides comprise one or more non-starch polysaccharides;
wherein said one or more non-starch polysaccharides comprise arabinoxylan and/or cellulose;
wherein said Bacillus strain composition comprises an activity of arabinoxylanase, cellulase, amylase, zein protease, and/or protease; and/or
wherein said Bacillus strain composition is formulated as pellets, mash, crumble, cake, meal, powder, or liquid.

54. The method of claim 37, wherein said effective amount comprises at about 1×10⁵ CFU/gram to about 1×10¹⁰ CFU/gram or at about 1×10⁵ CFU/ml to about 1×10¹⁰ CFU/ml of said bacterial cell, spore, forespore, and/or combination of cells, spores, and/or forespores of said Bacillus strain(s);
wherein said effective amount comprises at about 1.5×10⁵ CFU/gram of feed, forage, or fodder to about 1.5×10⁶ CFU/gram of feed, forage, or fodder of said bacterial cell, spore, forespore, and/or combination of cells, spores, and/or forespores of said Bacillus strain(s);
wherein said effective amount comprises at about 1×10⁵ CFU/gram to about 1×10¹⁰ CFU/gram or at about 1×10⁵ CFU/ml to about 1×10¹⁰ CFU/ml of said bacterial cell, spore, forespore, and/or combination of cells, spores, and/or forespores of said Bacillus subtilis 747 or active variant thereof and said Bacillus subtilis 750 or active variant thereof;
wherein said effective amount comprises at about 1.5×10⁵ CFU/gram of feed, forage, or fodder to about 1.5×10⁶ CFU/gram of feed, forage, or fodder of said bacterial cell, spore, forespore, and/or combination of cells, spores, and/or forespores of said Bacillus subtilis 747 or active variant thereof and said Bacillus subtilis 750 or active variant thereof;
wherein at least one of said Bacillus strains is a powdered, lyophilized strain;
wherein said one or more polysaccarides comprise one or more non-starch polysaccharides;
wherein said one or more non-starch polysaccharides comprise arabinoxylan and/or cellulose;
wherein said Bacillus strain composition comprises an activity of arabinoxylanase, cellulase, amylase, zein protease, and/or soy protease; and/or
wherein said Bacillus strain composition is formulated as pellets, mash, crumble, cake, meal, powder or liquid.

55.-61. (canceled)

62. The method of claim 31, wherein said Bacillus subtilis 750 is deposited under NRRL accession number B-68212.

63. The method of claim 32, wherein said Bacillus subtilis 747 is deposited under NRRL accession number B-67257, said Bacillus subtilis 839 is deposited under NRRL accession number B-67951, said Bacillus subtilis 1781 is deposited under NRRL accession number B-67259, said Bacillus subtilis 1999 is deposited under NRRL accession number B-67318, and/or said Bacillus subtilis 2018 is deposited under NRRL accession number B-67261.

64. The Bacillus strain composition of claim 13, wherein said Bacillus subtilis 750 is deposited under NRRL accession number B-68212.

65. The Bacillus strain composition of claim 14, wherein Bacillus subtilis 747 is deposited under NRRL accession number B-67257, said Bacillus subtilis 839 is deposited under NRRL accession number B-67951, said Bacillus subtilis 1781 is deposited under NRRL accession number B-67259, said Bacillus subtilis 1999 is deposited under NRRL accession number B-67318, and/or said Bacillus subtilis 2018 is deposited under NRRL accession number B-67261.
